SEQUENCE LISTING (24 sequences)

SEQ ID NO: 1 - DNA, 14544 bp, Homo sapiens
cgtactggtccatagcctgttaggaaccaggctgcataacaggaggtgagtggcaggtgagtgaaatttcatctgtagttacagccactcctcatcactcgcattaccaccagagctccactccctgtcagatcagcggcggcattagattctcataggagctcgaaccctattctaaactgttcatgtgagggatctaggttgcaagctccctatgagaatctaatgcctgatgatctgtcacggtctcccatcacccctagatgggaccatctagttgcaggaaaacaagctcagggctcccactgattctacacgatggtgaattgtggaattatttcattatatatattacaatgtaataataatagaaataaagcacacaataaatgtaatgtgcttgaatcatcccgaaaccatcccaccctggtctgtgaaaaaattgtcttccatgaaaccagtccctggtgccaaaaacgttgaggaccactgctccacagaatctatcggtcactcttcctcccctcacccccttgccctaaaagcacaccctgcaaacctgccatgaattgacactctgtttctatcccttttccccttgtgtctgtgtctggaggaagaggataaaggacaagctgccccaagtcctagcgggcagctcgaggaagtgaaacttacacgttggtctcctgtttccttaccaagcttttaccatggtaacccctggtcccgttcagccaccaccaccccacccagcacacctccaacctcagccagacaaggttgttgacacaagagagccctcaggggcacagagagagtctggacacgtgggggagtcagccgtgtatcatcggaggcggccgggcacatggcagggatgagggaaagaccaagagtcctctgttgggcccaagtcctagacagacaaaacctagacaatcacgtggctggctgcatgccctgtggctgttgggctgggcccaggaggagggaggggcgctctttcctggaggtggtccagagcaccgggtggacagccctgggggaaaacttccacgttttgatggaggttatctttgataactccacagtgacctggttcgccaaaggaaaagcaggcaacgtgagctgttttttttttctccaagctgaacactaggggtcctaggctttttgggtcacccggcatggcagacagtcaacctggcaggacatccgggagagacagacacaggcagagggcagaaaggtcaagggaggttctcaggccaaggctattggggtttgctcaattgttcctgaatgctcttacacacgtacacacacagagcagcacacacacacacacacacacatgcctcagcaagtcccagagagggaggtgtcgagggggacccgctggctgttcagacggactcccagagccagtgagtgggtggggctggaacatgagttcatctatttcctgcccacatctggtataaaaggaggcagtggcccacagaggagcacagctgtgtttggctgcagggccaagagcgctgtcaagaagacccacacgcccccctccagcagctgaattcctgcagctcagcagccgccgccagagcaggacgaaccgccaatcgcaaggcacctctgagaacttcaggtaggagaaaagcaaactccctccaacctcttacttcgggcttaaggcagagaactcgcctccccagaatctcctccctccatgatcccccgctattcctctattttcttttcctcggacctgcagccttgggtcgaccctgccctaggggtgactgcaggagagcagggaggatggtcaggcgtcaccaacaaccccatcacccagtaacaagaaccttgactctctcagtccctctgcatcaagacacttacccatttcccacctcatgcctgctaacttgaatgaaacaatcgctgggaaagcattaagagaattaaggctgggcactgtggctcatgcctgtaatcccagcactttgtgaggctgaggcaggcagataacttgagcccaggagtttgagaccagcctgggcaacatggcaaaaccctgctctcccaaaaaaatacaaaaattagctgggcgtgctggtgtgcctgtattcccagctacttgggaggctgaggtgggaggattgcttcagctggggaggcggaggctgcagggagccaagactgagccattgcacccagcctgggtgacagagcaagaccctgtctctaaaaatgaatgaaaggaaggaagaaagagagagaaagagagagaggaaagaaggaaggaagtaaagaagaaagaaagaaagaaagaggaaagaggaagaaagaaagaaaagaaagaaaagaaaagaaagcaaatttaaagcttatgcaaatcaaagatgttgtgataattgataattgagtctgggctaaattccccctgggctgcaaaggcagagagtggtaatgacttctcacctgcttttcttctaaggcttttttacgggacacagagggaagggagatggactggattccaagattcccacagggcaagatgggcgaagactccctgccactgcccggggataagtcagtctgagtgagacggagtgggatgggcttagaacctgaacatgtcatggtctcttcctgcaccttgccctagtgttcacttaccacctgcttgcaggaaacaagaagagcagggcccacagctggccagctcccctcccctcccgcctgtcttccagaacgattccttcaccagccctctttccattgctctaggatgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatggtgagccacgggacaccaggggaggtgggtggcatgcagaacagacctaccagaagccaaggaaaggctggctctggcttagccgagccaagccccatacagctgtgctgcaggggccaccccatcttcttcccactacactccaagtcactggacccttgaatctccaagggtgtctgaccagtagatttaccgcttattcaccaccgtgtgatcttaacctcgttaagtttgcccatctacaaaatgaggattatttgctgtcctaaagaattcatgagccgggcgcggtggctcaaacgcctgtaatcccagcactttgggaggccaaggcgggcggatcatgaggtcaggagatcaagaccatcctggctaacacagtgaaactccatctctactaaaaatacaaaaaaaattagccaggcgtggtggcaggcgcctgtagtcccagctactcgggaggctgaggcaggagaatggcatgaacccaggaggcagagcttgcagtgagctgagatcgtgccactgcactccagcctgggcgacagagagagactccgtctcaaaaaaaaaaattaaaagaattcatggaattacacttgtgaaatacttagcatagccatcactataggaaaaaaatctaaggccaggcacagtgcctcatgcctgtaatctcagcactttcggagtttgaggcaggaggatcacccaaggctaggagttcaaggccagcctgggcaatacggtgaaaccccgtctctaataaaaatataaaaattagtctgatgaggtggtgcacctgtaatcccagctactcaggaagctgagacacaagaatcactttaacccgggaggtggaggtggcagtgagctgagatcacaccattgcactccagcctgggtgacagagtgagacctgtcaaaaaaaagaaaagaaagagagagagagagagaagagagagaaagaaagaagaagaaagaaagaaagagagagagagaaagaaagaaagaaacaaagaaacaaagaaagaaaggaaaagaaaaaaaaaactaaggccaggcaaggtggcttatgactgtaatttcagcactttggaagattgaggcaggaggatcacttgaggccagaagttcgagacaagactgagcaacagggagacccctgcctctacaaaaaaatttacaaattagccagatgtggtgacacatacctgtagtcccaactactcaggaggctgaggtgggaggatggcttgagcccaggagctggaggctgcagtgagctatgattgtaccactgcacttcagcctgggcaacaaagggaagccctgtctgaaaaaaaaaaaaaaagaaaaagaagaagaaagaaaatatttagggttcatccaggaggcagaggttgcagtaagctgacatcgcgccattgcactccagcctgggagacaagagcaaaactccaactcaaaaaaaaaaaaaaaaaaaaaacaggaagaaaatatttagggttcataacttaagaacagagaaaaatattctagcccaaagaaagggttgggatctgagacttttgaagaaaggaaggagatacagaaaagagatttcatcctggaatgaaatctccctccagagagccctgggaaagcacggtagcccccatccatcagagtggagccccttgtgggggaagtgggctcggctgggaaccctcaattcagcataagcctcacatgtcctctcctctctgtcccggtgcagacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtgagcaggcagggaaaggaaacccatttcctgggcctcaagagaaagggaatttggaaataaatccacatatcccagttgggtgcagtagttcacacctgtaatcccagcccaacactttgggaggtctaggcgagaggaaggcttgaggcctggagtttgagaccagcctggccaacataacaagacctcatctcttcaaaaaatttaaaaaccagccgggcatggtggtgcacacctgtagtcccagctacttgggaggctgaggtgggaggatcacttgagtccagcagttcaaggctgcagtgagctatgtttgcaccaccacactccagcctgagtcacagaacaagacctcatctctaaaaaacaaacaaaaaccaaatccacatatcctaaaaaatgctccttttcagcattctcttctctatggacaaagggctggatgctttaagaaccaaatcttaggctgggcacggtggctcacgcctctaatcctagcactttgagaggccaaggcgggcagattgcctgagcacaggagttcgagaccagcctggccaacatggtgaaaccctgtctctgtcaaaaatacaaaaaattagccaggtgtgttggcgcgtgcctataatcccagctgctcgggaggatgaggttcaaagaatcacttgaacccgggaggcagaggctgcagtgagctgagatcatgccactgcactccagcctgggtgacagagcaagactttgtctccaaaaaaaggaactagacgggttcatttaaacccctgactgcagccctttgacatacatccaattgaggactggggactccgggaaacatctaaaaggcttaaaaactttgtctaacttcagccgggcatggtggctcacacctgtaatcccagcactttgggaggctgaggcaggtggatcacaaggtcaggagtttgagacgagcctgaccaacatggtgaaaccccgtctctactaaaaatacaaaaattagccaggcatggtggcaggcgcctgtaatcccagctattcgggaggctgaggcaggagaattgcttgaaccccggagacagaggttgcagcgagccgagatcgcgccactgcactccagcctggcaatagagtgagactccatctcaaaacaacaacaacaacaacaacaacaacaaaatcgtctaacttcctgatcttcctgatcattgattttcccataggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatagtaagtccaagagccccttccccacagcccacagcaactgcatctcattcctggggtctcccaaggaatacccaaaatgtcaccctctgagggaggaagaccacagggaatgctcccctttaagggaggagagaccctagaatatactccagctttgacaaagatttcccaagcaggagacatcaggataatgggaacagaagacaggaggtttatcccatgaaggatgaagaagctgaaatccagagattccctcagggccacatttgtccacctgactccagggtctcatcttcgtgtgttgctagtgtgattacctggggatgagaaatcctgctgggggagttgaggttaagaggatgaggactccaggtgctgtggctcacgcctgtaatcccagcactttgggaggccaaggcaggtggatcaggagtttgaggtcaggagtttgagaccagcctggccaacatggtgaaaccctgtctctactaaaaatgcaaaaattagccaggtgtggtggcaggcgcctgtaatcccagctactcgggaggctgaggcaggagaatcacttgagcccgggaggtggaggttgcagtgagccgaacgaaattgagccacttcaccccagcctgggcaaaagagtgaaattccattcaaaaaaaaaaaaaaaaaaaaaaggatgaggactgggatgaactggtggctgggtgtggggaaaatggaagtgaaggaaggccaaaagagacagagaaggcctggcgcggcgactcacgcctataatcccagcactttgggaggctgagaagggggattgcttgaggccagaagttgaataccagtctgggcagcatagcaagaccctgcctctacaaaaaaaaaattttttttaattagccaggcttggtgacatgcatctgtagtctactcaagaagctgaggtgaggccaggcacggtggctcacgcctgtattcccagcactttgggaggtcaaggcgggtggatgacctgaggtcaggagttcaagaccagcctggccaacatggtgaaaccccatctgtataaaaatacaaaaattagctgggcatgatagcaggtgcctgtaattccagctactcaggaggctgaggtgggagaatctattgaacccgggagggggaggttgcagtgagccgagatcatgccattgcactccagcctgggcgacagagtgagactccttctcaaaacaaacaaacaaacaaacaaacaaaatacagaagctgaggcgggaggaacatttgaaccggattcggaggctgcagtgagctatgattgcaccactgcgctccagtctgtgtgacagtgagaccctgtctcttacacacacacacacacacacacacacacatgcacacacacagagagagagaaattagaagatactgaattggcagaagagaagggaaatagaaattaaaatactgaataggggagcagtgaacaggggatacccaaaagccaagagcgagagagagcctggcttccagaaatagtggagaagccaggagaactaggtgaaaacccagtgctgggttgccatcagcgagagctggagccatttccaacgaaccatcttgtcgtcttcacagctgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagtaagccaccccgctatctccccgacctaccaacccctctctcctggctccctaaagtcaccgcccccaggttgaatttcccagatctgtgatgcttgcaggacatgcatgtgtgggaggctgatgggaaactgtggcctgggtttgattatgagtcttgcaatcatccctccccctgtttctgctggagggcaggggacagctcttcctgaccacacccccacattgactatccccagaatacccagcaaaagcccccaaaaggagagtcagagaaatgagggaggtgggggcccaatcagtccacatctacttagggtcgccccatcagcacttccatccccaaccctttcaagtcaacatccaaacaaaagaaatcacttccaaggacggagcagctcaaagcgcagcttctagctggggttccaagaaagcagatttttcgaaatccttctgcagaaggaagcaaagagattttttgaaatctttctgcagaaggagaaggctggagctggggaactccagaattatagggaagcctcccaccacgctcatcccaaatttccggatgctataatgccaggcttggggaaagaggagaatttagttggttagctggtgcgtgctctcacttgcatcctctctcttcctcttttttttttttctcctctctctctggctcataaaaatggaggtaattagttgtgccctggtgagaagcagagagtgcacaaaggccccctgcttgagtcctcttcagggttagctctcagaaacacaatctgcagaacagatttttgttccaacatccttgcaggagaatttgcccttagcttcccccaccccagccaggctgaataaaattatgctgaaactactgtcttatttgaggaaagtaattagtcataggtgggagggggtggggagattgcagaagaatgttcatgaatattaggattttcagctctaaggggggactttgtaaacagctttagaagaagaaccaggccggctgggtgtggtggctcatgcctgtaatctcagcatttggggaggccaaggcgggcggatcacttgaggtcaggagtttgagaccagcctggccaacatggtgaaaccctgtctctattaaaaatacaaaaattagccagccgtggtagcgagcgcctatgatcccagctactccggaggctgaggccagagaatcacatgaacctgggaggtggaggctgcagtgagccgagatcacgccactgcactccagcctgggggacagagcaagaatctgtttcaaaaaaaaaaaaagaaaaataggaaggaaggaaggaaaggaaaggaaagaagagagagagaaagaaagagagagagagagagagaaagaaagaaagaaagaaagaaagaaagaaagaaagaaagaaagaaagaaagaaaaagaaaggaaagaaagaacgaacgaaccaggcctccctctccaaccttcacctccgtccctattctggccacttgattcgggggacacctggtaggggatggggaaaggtgggagctgccagccagaggggaccccggcttgagcagcctcttgctgctatctgcaggttctccctggagactgaagtcgacctcaggaagcccctagagaacctgggaatgaccgacatgttcagacagtttcaggctgacttcacgagtctttcaggtaagaagactttcctttgcattttctcaccccagtggactgcgggggcccctaagaggaaaaaggaacctctccttgagagcggcagctgatctaatcctgtatccacatctgtttcagaccaagagcctctccacgtcgcgcaggcgctgcagaaagtgaagatcgaggtgaacgagagtggcacggtggcctcctcatccacaggtgagtctggctcaggtgaggctccacgggtgtcgcctccatcgcccttcaggataactggtccccagacccggaaaggaccccgcagccctctcggcacagagcagctctgtctgtgctcagccatcacccactccccacctgtttctcagcctggaaaacgggcttgggaccatggaaccctgtttcctcgcctgatggctcctaagttccctgactgtgaaaaggcctcctaaagaaaaacccaagttgttcccacagtgggaagtaaacttaagaaacatgcttatcaggctgggcatggtggctcccacctgtaatcccagcgctttgggggaccaaggcaggtggatcacttgaggtcaggaattcgagaccagcctgggcaacatggcaaaaccctatctctactaaaaatacaaaaattaggcaggcgtggtggcatgtgcctgtagtcccagctacttgggaggctgaggcaggagaatcacttgaatccaggaggcagaggttgcagtgagccgagatcacgctgctgcactccagcctgggcaatagagcatgactctgaagaaaagaaagaaagaaagagagagagagagaaaagaaagaaagaaagaaagaaagaaagaaagaaagaaagaaagaaagaaagaaagaaagagaaagaaagagaagaaaagaaaagaaagagcttatcaataagcccttaaaggatttagataaatgtgtgtaagggaagagctgatccattgctaccaagctcctggaggaaaccaggtctcagaggatgtccctaaacttttaaggttcatattcaggaaaacaaacaacttccagctgggcttagtggctcacacctgtaatcccagcactttgggaggccgaggcaggaggatcgcttgagcccaggaatttgagaccagcctgggcaatataatgagactgtgctctacaaaaattagaaaaaaattagccaggcatggtggcatgcacctgtagccccagttacttgggagactgaggtgggaggatcacttgagcccatgagttcaaggctgcagtgagccatgaaggtgccactgcactcccgcctgggcgacagagggagaccctgtctctaagaaaaacggcgggggtgggggtggtgccagtgccagcatccctctgttctaagacattgtcccttctcttgcagctgtcatagtctcagcccgcatggcccccgaggagatcatcatggacagacccttcctctttgtggtccggcacaaccccacaggtgagcctggaacccatcacgttccacatcctcccacccattctttctctcaggaactagtcccgacagatgcagacatccctctatccctgagagggctctgggcagggaaccataaccctaccctgcttcctgtcccaagaggaggctaccttctatcacccacagacagtgccgggtccccgctctgtgactcaggcagctgcgactccagacagctcactcatctgcctagatctcagtccttccacccacatccagcctgatgagctgtcccactccttctgcttctcaacccccatggttcttccaccctcaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggatggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaatgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccgtatctcaggaagtccagccacttgactggcacacacccctccggacatccagcgtgacggagcccacactgccaccttgtggccgcctgagaccctcgcgccccccgcgccccccgcgcccctctttttccccttgatggaaattgaccatacaatttcatcctccttcaggggatcaaaaggacggagtggggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagtggacacgtgagggggggggtgggtgagagagacaggcagctcggattcaactaccttagataatatttctgaaaacctaccagccagagggtagggcacaaagatggatgtaatgcactttgggaggccaaggcgggaggattgcttgagcccaggagttcaagaccagcctgggcaacataccaagacccccgtctctttaaaaatatatatattttaaatatacttaaatatatatttctaatatctttaaatatatatatatattttaaagaccaatttatgggagaattgcacacagatgtgaaatgaatgtaatctaatagaagcctaatcagcccaccatgttctccactgaaaaatcctctttctttggggtttttctttctttcttttttgattttgcactggacggtgacgtcagccatgtacaggatccacaggggtggtgtcaaatgctattgaaattgtgttgaattgtatgctttttcacttttgataaataaacatgtaaaaatgtttcaaaaaaataataaaataaataaatacgaagaatatgtcaggacagtcactgccttcaccttctccatttcacaccggtggtacaagaaatcagaagcctaggccaggtgtggtggttcatgcctgtaatcccagcactttgggaagccgaggtgggtggatcacctaaggtcaggagtttgagaccagcctggacaacatggtgaaaccccgtctctactaaaaatacaaaaattagccgggcgtggtggctggcgcctgtaatcccagctactcgggaggctgaggcaggagaatcacttgaagccaggaggcagaggttgcagtgagctgagattgcaccactgaactccaggctgggtggcagagcgagactccctctcaaaaaacaacaactacaaagacaacaacaaacccagaatcaaaatcctgttggtccatagacctcatgggtggaagagaccttcctacatccaggttggcccaacatgggggagtccatgaaatggtcacctcagctctgccacaagccccaaggataagttgattctgcccctgggaatcatcctcaaaaaggaaaaaaatgttcccctgccataaactttccacttatgcagatgggcctgctcgtaagtcactgtcactgtgggttcccaactctgttcatgacacttccttccagcaccaaatgcttcccacccctctactcccactccccattcttcaaacccagctcaagttccagttcctccacctaggacttcccatggatccagccaatatcactctcaggtccggcgcagtggcccacgcctgtaatctcagcactttgggaggccggggcaggaagattgcttgaggccaggagtttcagaccagcctggacaacatagtgagactc

SEQ ID NO: 2 - DNA, 20 bp, Artificial
  Synthetic Primer
gtgggggagtcagccgtgta

SEQ ID NO: 3 - DNA, 19 bp, Artificial
  Synthetic Primer
taacccctggtcccgttca

SEQ ID NO: 4 - DNA, 19 bp, Artificial
  Sythetic Primer
ttttcctttggcgaaccag

SEQ ID NO: 5 - DNA, 21 bp, Artificial
  Synthetic Primer
ccagacaagcttgttgacaca

SEQ ID NO: 6 - DNA, 20 bp, Artificial
  Synthetic Primer
acctccatcaaaacgtggaa

SEQ ID NO: 7 - DNA, 18 bp, Artificial
  Synthetic Primer
gacacgtgggggagtcag

SEQ ID NO: 8 - DNA, 18 bp, Artificial
  Synthetic Primer
ggacacgtggggagtcag

SEQ ID NO: 9 - DNA, 1723 bp, Homo sapiens
gttgtacagaaaattgctgttgggatgaagctttgcagccttgcagtccttgtacccattgttctcttctgtgagcagcatgtcttcgcgtttcagagtggccaagttctagctgctcttcctagaacctctaggcaagttcaagttctacagaatcttactacaacatatgagattgttctctggcagccggtaacagctgaccttattgtgaagaaaaaacaagtccatttttttgtaaatgcatctgatgtcgacaatgtgaaagcccatttaaatgtgagcggaattccatgcagtgtcttgctggcagacgtggaagatcttattcaacagcagatttccaacgacacagtcagcccccgagcctccgcatcgtactatgaacagtatcactcactaaatgaaatctattcttggatagaatttataactgagaggcatcctgatatgcttacaaaaatccacattggatcctcatttgagaagtacccactctatgttttaaaggtttctggaaaagaacaaacagccaaaaatgccatatggattgactgtggaatccatgccagagaatggatctctcctgctttctgcttgtggttcataggccatataactcaattctatgggataatagggcaatataccaatctcctgaggcttgtggatttctatgttatgccggtggttaatgtggacggttatgactactcatggaaaaagaatcgaatgtggagaaagaaccgttctttctatgcgaacaatcattgcatcggaacagacctgaataggaactttgcttccaaacactggtgtgaggaaggtgcatccagttcctcatgctcggaaacctactgtggactttatcctgagtcagaaccagaagtgaaggcagtggctagtttcttgagaagaaatatcaaccagattaaagcatacatcagcatgcattcatactcccagcatatagtgtttccatattcctatacacgaagtaaaagcaaagaccatgaggaactgtctctagtagccagtgaagcagttcgtgctattgagaaaactagtaaaaataccaggtatacacatggccatggctcagaaaccttatacctagctcctggaggtggggacgattggatctatgatttgggcatcaaatattcgtttacaattgaacttcgagatacgggcacatacggattcttgctgccggagcgttacatcaaacccacctgtagagaagcttttgccgctgtctctaaaatagcttggcatgtcattaggaatgtttaatgcccctgattttatcattctgcttccgtattttaatttactgattccagcaagaccaaatcattgtatcagattatttttaagttttatccgtagttttgataaaagattttcctattccttggttctgtcagagaacctaataagtgctactttgccattaaggcagactagggttcatgtctttttaccctttaaaaaaaattgtaaaagtctagttacctactttttctttgattttcgacgtttgactagccatctcaagcaactttcgacgtttgactagccatctcaagcaagtttaatcaaagatcatctcacgctgatcattggatcctactcaacaaaaggaagggtggtcagaagtacattaaagatttctgctccaaattttcaataaatttctgcttgtgcctttagaaataca

SEQ ID NO: 10 - DNA, 24 bp, Artificial
  Synthetic Primer
cacaaagaaaaacagatcacacag

SEQ ID NO: 11 - DNA, 20 bp, Artificial
  Synthetic Primer
aaagccacccaattgtgatt

SEQ ID NO: 12 - DNA, 18 bp, Artificial
  Synthetic Primer
atggctgtgtctggggcg

SEQ ID NO: 13 - DNA, 18 bp, Artificial
  Synthetic Primer
atggctgtgtctggggca

SEQ ID NO: 14 - DNA, 18 bp, Artificial
  Synthetic Primer
attggcgcaaactcctca

SEQ ID NO: 15 - DNA, 18 bp, Artificial
  Synthetic Primer
gcaggtgggcctaaagcc

SEQ ID NO: 16 - DNA, 18 bp, Artificial
  Synthetic Primer
gcaggtgggcctaaagct

SEQ ID NO: 17 - DNA, 19 bp, Artificial
  Synthetic Primer
ggctcagagtctttttggc

SEQ ID NO: 18 - DNA, 20 bp, Artificial
  Synthetic Primer
cttcagcaaacgtaccatgc

SEQ ID NO: 19 - DNA, 26 bp, Artificial
  Synthetic Primer
ctaagctgtttgatgggtatcttctc

SEQ ID NO: 20 - DNA, 26 bp, Artificial
  Synthetic Primer
ctaagctgtttgatgggtatcttctt

SEQ ID NO: 21 - DNA, 23 bp, Artificial
  Synthetic Primer
ggcagcttcaactcagctccagt

SEQ ID NO: 22 - DNA, 23 bp, Artificial
  Synthetic Primer
cgcctcatttctccctcatctgc

SEQ ID NO: 23 - DNA, 9781 bp, Homo sapiens
ggatcctcttatctcagcctcccaagtagctggaccacaggagcaaaccaccatgcctggctaattttctgtatttttttgtaaagatgaggtgggtctcactctgttgcccaggctgatcttgaactcctggcttcaagcaatccctcaccttgaactcctaaagtgctgggattacaggtgtgagccaccttgcccagctcacttctgtaataagttgagagagaggtatatatagagagaccacagtcacataacttttattacagtaccttgttatgattgttctattttactattggttattgttgttagtctcttgctgtgcctaatttatacattaactttgttttgttttgttttgttttgtttttgagacacggtctcactctgtcacctaggctggagtgcagtggcacgatctcggctcacttcagtcttcgcctcctggattcaagctattctcatgcctcagccttccgagtagctgggactacaggcacacaccaccacacccggctaagttttttgtatttttagtagagacagggtttcatcatgttgcccaggctggtctccaacggctgacctcaagtgatccacctgcctcggcctcccaaagtgctgggattacaggcatgagccaccatgcctggccacattaaccttgatcataggtatggatgtgtagggaagagcatcgtatatatagtgctcggtactacccacagattcaggcatccattgggatcttggaacacatccttgtggttaagggggatgaccatgtgtgatggccctcccagctccctgcctcctgctcctggagtctccatggctcctcagcgccaatcctccctgtctttcagccctttgccctgtcagcctagactcaatgacccattgtgaaccattaccacgatgcccgttgacatgctggaaactcttcccttcttattctaaaccccaaggccctgtcaacacgctctactggggaagcacacctactgctttggtttccaatatgatttcagagcaattgccttgcaaaccttcagcaacaacctctcctgtgctgttaaccctttgaaagagtgtgcatttttaaaaagttggcattgggcacagtggctgaagcctgtatcccagcactttgggaggccgaggcaggtggatcacctgaggtcaggagttcgagaccagcctggccaacatggcaaaaccccgtctctactaaaaatacaaaaattagctgggcatggtggtgggcacctgtaatcccagctactcgggagggtgaggcaggagaatcacttgaacccaggaggcagaggttgcattgagccgagatcgcatcactgcactccaggcagggtgacagagcaagactctgtctcaaaaaaaaaaaaaaagttggcatcaatgtgttcttccatctgatttatgtcttcacattacagcatgttttctgtatgagcccacacatatcagatggtaactttagaatgggtgggtctgtgccattgtcccggcacagctgtgagggtggtgacaggcaggtcagcaggtgcgtacagaggggctttcttctacattggagttcaggtcggatgatccaggtccacagtgttcattcagggctctgtgttctggtggcttccgttgatttctaataaaagctgcagactgagccagtcctgcacacacagggagataggacaacgaacatttgggatgtgattaagcccatcaatccaatcggtgactaaaatcagacaggaagccctgtgccccttagataaagaaagcctggggggaaaatgatctcaagttcatttcgattggcgcaaactcctcacagaggaaaaaatgtacagttagagtgaagtgaaagaacatctctataaaatatgcatcacttcctggcggggaggagagaggagctatggaaagctacaccaaagctgtattcactggacaaaaatgcttgactcaggaaggaggccggagcggcgagtcctgtgatgccatggcgggaggtgggtcccatgtaaacagtggtgttcctgtcaccctgagcacatgcagtctcccgtgggtaaccagaactgatgcaagagcccctgctgtggaagtcaccacgctctcccagaacgcgcctccccccaggtctgagtgatctcattgccgaggtgaatagggctttggccgctctcccaaaggagcccgccccagacacagccatggcctgggactctggggtcaccctggggtcagaaggaattatctgtattcacttggttttggttattgtcagtgttatttatgtgtttatttattttttatttttagagacaggcttttactctgtctcccaggctggagtgcagtgatgggatcacagttcactgcaacctcaaattttcaggttcaagggatccttccacctcagcctcctaagtagctgggactacatgcatgtgccaccacacctgactaattttttaattatttgtagagatagggtctcgccatgttgcccaggctggtctcaaacttctgggctcaaggaatcctcccaccttggcctcccaaagtgttaggattacaggagtgagccacctcacctggcctggtgttgttattttaaaacaagaaaagaaatgaccagaaaggctgaaaggaaacacgcaccatcctccataggaggctgcaagggttggtggcaccaggtccagaagaagaagggctcatagagcagaaaccagagcttgagcaccacctgggtggcatttggaagtgcctcagcacagccactttgcagagtttgcacatttaaaataaggagtaggctgggcacagtggctcatgcctgtaatctcagcactttaggaggccaaggcaggcggataccttgagttcaggagttcgagaccagcctggccaacatggtgaaaccccgtctctataaaaaaaatatataaaaattagccaggcgtggtggtgtgtgcctgtagtcccagctactcaggaggctgaggtgagagaatcacttgaactggggaggtggaggttgcagtgagccaagatcatgccactgcacttcagcctgagcaatagagtgagatcctgtctcaaaaaattaattaattaagttaaataaagagtagcgcaattttcctaaacagattgaccagagacacccttaaaattatacgactgtgatagaattcctacagatattcattagctatgatatgttggataaataagcagatggcagtgaatattcttacagaccaaatatttctgcaaataaaattaaattcttatgtaaaatatgcatacataaatatattctagatataattttataatatcagaaatgatcatatttttcatttgcaagtactttcttctttttatttttagagttgggggtcttgctctgttgcccaagctgtactgcagtggcatgatgatagctcactgcagccctggactcctgggctgacgtgatctgcctgccccagcctcctgaatggttttttcttttttttgagacggagtttcactcttgttgcccaggctggagtgcagtggcgcgatctcagctcacagcaacctccacctcccaggttcaagccctgaatggctttttaaaaatattttctcagagtcagggtcttgctctgttgcccagactggtctcaaactcctgggttcaagcgatccccctgtcttggcttcccaaagtgctgggattatagatgttagccactgcctcctgtcaatattttttaaattaatagacattccttagagcagtttcaggtttacagaaaaattgagcaggaagcacagagttcccatattattatcccttctcttccctccgcagtttcccctgttattatcttgtacttagtccagtgatattgttttttttttaaactactattgtttttcagtttttagatagtcaaaacaaagttgtagacctatcattatcttgtatgggaatttaaaaaatgtatttcactgggctaatgtattagtccgttctcatggtgctgtaaagaaatgcccaagactgggtaatttataaagaaaagagatttaatggactcacagttccccatggctggcagagcctcaggaaacttacaatcatggaggaaggcacctcttcacatggcggcagaagacagaatgagcacaaggaggggaaatgctagacgataataaaaccatcagatctcatgagactcacagcttggggaaaaccgccttcgtgatgcaattacctccacctgtttctgcccttgacctgtagggattatggggattaaaattaaaggtaagatttgggtggggacacagagccaaaccatatcaactaaatattcaaaaaaagtactgctctaagagagtacttcaatttttaacttggtagccttggttcttgttcatgtgtgtaacactaacatagcttaggaggcagttggaaaaaatttgagtcaggggctctttaaagttcctttcttcatctttgagaaaacagtacgaggctttcccttgcaaagttctttccatctttacttctcttatctgaaacgcaaggaacctatagcttatcaaaggtaaatggactggaccagagtcgtgcaactgacctcagaggagagaaaggaaactttctgacttccaaccccaggctcattccagtagcctgcacattcaccacgggggtgtatttgaagattgatagctggcgatgtgttaggaggctcaaaagaggtagatttaggagcagagaaagaaagttctgtgtcggagaatcagcaaaccaacctgagaaactcaactcactccacaaaggaattgattgaaaattagatgtgttcaaaaataacttgaataaactcatggatactccatcataagttattaaggaaaattcctattcttctgacacgcttccatgaacaaccaggtccttccagtgccagagagagagtgttgggctgggcacacccagtgtggagccagtacatctacttttatgaaagtcatttagaagcatcactaaaggaactgaaaaacaattcaccaataatgacagtactccaacacctagttttatcttctaatctctatccatacatctatgtagtgtttatattattattattttttgagatggagtctcactcttgttgcacgatcttggctcactgcaacctccacctcccaggttcaagtgattctcttgcctcagcttcccgagtagcggggattacaggtgcccgccaccatgcctgactaatttttgtgtttttagtagagatggggtttcaccatgttggccaggctggtctcgaactcctgatctcaagtgatttgcctgcctcggtctcccaaagggctgggattatgggtgtgagacaccatgcctgatctacattatttatatcacagctcatgtacaaatttattttcttcttcttttgggcaaggcaattttatgatttattttggtatgtcggtataattatgttatcagtcatctcaatggctacacaatattgcagtattctaacatgtcctagtctactaggcccctatgttttgacatctaagttattttcagtatttagacagtcaaaacaatgttgaatacttatcattatcttgtatggggatttaaaaaatgcatgtcagcaggctaaatattcaaaaattactgctccaagaaaataatccaatttttaacttaatattttgaaataatttcaaacttaccaaagagttgcaagaataaagacctcctcctacagtttggtgaacagctcttaacaagaataagatttgtaagagtccttcccatgccttcagcttgtcctgttttgatgtttctcattgtttgtgtggaattcacacaactggtgctgttaccaccatgggcgtctagtctggatcagtggtcctcagtcttttttgcaccagggaccagttttgtaaagatagcttttccacggacagagggaggggagatagtttcgggatgattcaagaggattacatttattgtgcactttatttatattactattacattgtattatataatgaaataatggtatgactcagcataatgtagaatcagtgggaaccctgagcttgttttcttataactagatggtcccatctgggggttatgggagacagcgacagatcatcaggcattagattcttataaggagtgcacaacctcaatccctcgcatgtacagttcacaatagggtttgcactcctatgagaatctagtgccactgctgatccaaccggaggtgcagctcaggcagtaatgcgagtgatggggagcggctgtaaacacagatgaagcttcactggctctccagccactcacctcctgctgcgcagcctggttcctaacaggccacggacagataccagcccatggccccagggccggggattcccagtctagatggagacctagacaaggcgtgcgacaataacaccgattttagatccatcatgacatttaccccatcccctgcaaagccagatggctaccaaaattaaatcttagtttagacacagaatgtccgtcttctggtccaaaacatccttgtcataagtttcttggctgggcgcggtggctcacacctataatcccagcactttgggaggctgaggcaggtggatcacgaggtcgggggtttgagaccagcctgaccaacatggtgaaaccccgtctctactaaaaatacaaaaaaattagctgggcgtggtggcgggcacctgtaatctcagctactcaggaggctgaggcaggagaattgcttgaacctggtggaggttgcagtgagccgagatcacaccactgcactctagcctgggcgacagagcaagactctgtctcaaaaaaaaaaaaaagaaaataaaaaaaaaaaaacaagtttcttgcccactcttcctttctctgagtttccagagacatcacatcatttcttacccagctgagcagagtcccagcatggctctcgttcgaatacccatcctgccacctgccccagtgagaagggttggagcagcccctgttctctgcccccgccacctccatgactcatgcattctgtgggggaggcgcccactgcagaaacagccaggctggctgggaaaagccctgcagcaattccccgtccagtttctctgtgcccactttgtctccgtgttatctaggcctggatttatttctcttttttgaaaatgaagggctttgatgaaatgttcacaggatgtgaggtcaccaagatttttttcctttttttctgttcttttcttttcctcgaagtgatcccttgactaaaatcaaggctcccattgtcaccttatcagcctgcccatgcctaattctgcattctcaggcttccccagaatctgtccgagggaaaccaatctcaattcagaaggaacacagaggccccagtttctagggctgcaggatattgctgggtcttaatcattcgggtatatttcagcaaagctcagggcccctgctaatgatctgcaaaccctctcctctcagtctcccgttgtttagcccctatttataagtgagtgcatgtggttgttcactttctgtttctgagttatttcagttaggataatggtctccagtttcatccatgttgctccaaaagacagacttcattgttttttacggctttgtcgtattcctcggtgtatatgtgcattttctttatccagtcctccattgatggacacttaggttaattctgtgcctttgctattgtgaattgtgctgttgtgaattgtgtgtgataaacgtgtgagtgcaggtgtctttttgatgtaatgatttctttttctttgaatagatactcagtagtgggattgccgggtcaaatggatgttctatttttagtttgggaaatctccatattgttttccatagaggttcaggatccaggatttaagtgcaaccattcagtggaaaaccatcctctcaagagttcaaatgagagtcctcatgaccagcactccctgctgaacggcaatgccccaccccccaaaaaagcctccatcctcaccatcttagaccacattgcaggaaagcagccccatgcccaccagacaggcccacagctgctgctgctcatgggaggtctacctcccgtgctcagcaggcaacccaccccagtgccccttttctacaatgaccatcagcagggctgcagcttcataaaaatgtgaaaaacagtttgaaagcaatgtgaaaagcagttaggtgcctttcaacttcaaactcagcactcatggtttgacaaatatgagtatatttaggatcagaatgtatgaaatctggcctctcctgaatagtcggtcatcccacaatttcctctgccaaacttctcttccctccctgcgttctttctttcacctctctctcatttttacaaggtctggtctcagccagacatgaaccaatgatgatagatgctcctgctgagccctgtgatgtgccagggccctgacatgcgtggcttctctctgatcctcacgtgacctgcagatgcaaaagccagagctcagcatagttgaaaatcttgattgaggtcatatcccagttactggcacagccggatttaaacctaagactttcccctacacgacagggcttttatttctcagtcatctgaaaaggtgtcagcaagggaaatggcttgtctatttccaggggcattttacaagcaaatactgaaaggcttcggtgggcttaagggctgatggctttgatcgaatttcaggcatgttggccccaaggccctgtgtatattccctgggcccactcaaggggatgctggagccggaaagtccccggaggccacctactgcagccctgcactttacaaagaagagaaagattctccctaaaattacagaacagggccaaagatgcctaccggagcaaacccccatgggggcacctcctaccgcaggtgagcccaaggctggtcctgccttctcagtggctacccccctgagctcccgcaccacacaaagtgttccaatccttgtgcatcctccagtccttttaacctctcatgtcctgagaggccagagctacagccacagattccagaagacaccccactcccagccccaacctgctgcctttagaattataaacacttcttgtcatcacagggtcctgaaagtcccttttaagcctgggacactaggactctaaaggaagatgattcttaaggtcccatcccacttccaaattcctgcgattcaatgacatcacggctgtgaataatcagcctggcccgaagccaggatgggctgtgctgcttccaccgtgaacttcctccccctgctttataaaaacaggcctgcctcagctccctcatggccctgtccactgagcatcctcccgccacacagaaacccgcccagccggggccaccgaccccaccccctgcctggaaacttaaggaggccggagctgtggggagctcagagctgagatcctacaggagtccagggctggagagaaaacctctgcgaggaaagggaaggagcaagccgtggtaggtcgggtttctgtaccttggggtctgtctcctcttctttctcttaaaagtctttccagcaagctgagccagtgaaggaatgctttaaccaagggaccaactttgtaattcaggcaactagaggatcc

SEQ ID NO: 24 - DNA, 10075 bp, Homo sapiens
taatgggttctcaaatccaaacaaaactgaccacagtcttctagaggaagtagcaaggttggctctgaagcctatagcatctctgactcagtctgtcccctggaaggctggcagctcagcaagcacagaagtctctccagaagacagtgggtcacctgcctcccaaaagctgaaaggctaacttgtactttccccagcaggcagctggcatcctgagccctcggctggggcagagcaaaggagccttcctccttcctaccttcctggcactctccctgccttccttctgtcactctcaggtggacccagacccaaggtccagatttgcaaggcaggaaaatgctgcaggcctaggctgggaaagggcccaaagccgctagtggattgctgggactcagcctcctccttcccactaagagagcgagtcctactgggttcaaaatgaccccaagccctggttcctgacactaggggaaagagatgggggtgacagaatcacagaatccctgctatgttcctccaagtgtgcccagagatgcgtgtgtgtgtgtgtgtgtatacacaaatgtctgcttatcctcaggcaggaagggtggatgcagtcatttacacatggtctgtttttctggaggacaattttatttgataaacaattgtttctatctgaatagaataaacaaggctctatgatgaagtaaaacactaaatacacatgcattaaaaaatgcataattatctttttggaatgggctatacagagatgtgctttttaaaatgttaagagtgtaaaaggacaaacagtgaaaaataaatcttcctcttattttgtcctccagtctcccaattcctctactcagaggtgagaacagaacttccacaccctccagaacctccacagttagaactgtctacatgtttccattgtctttacttttattcttgcctgcacaaataaatgaattgctcattatggaaacttcccaaaagacccgttaacacttcaataggaagcaccaacagtttatgccctaggactttgttcccacaatcctgtaacatcatatcacgacacctaacccaatccttatcaagccctgtcaaaaacggactttaaaccaagctgcaaattttcagtaatctggccttgcctttccccctctgatagcaccatcaaacaaacccccttactgccgaaagcaataagcccggctttgttccatccactggttgtgttggtgatatctggggactgccactgaacagacgcacagagggagcccctacaggcagggggtttttctgtctgtgcttctgggagagtatgtctcgtacatttgtcgcgttgatgaagacttcacagctccatcagctgcgggcaagggggtctgaggcagtcttaggcaagttggggcccagcggggagaagttgcagaagaactgattagaggaccccaggaggcttcagagctgggcgaggtagagagtctcctgtgcgccttctctcctctctgcaattcggggactccttgcactggggcaggcccccggccaggtgcatgggaggaagcacggagaatttacaagcctctcgattcctcagtccagacgctgttgggtcccctccgctggagatcgcgcttcccccaaatctttgtgagcgttgcggaagcacgcggggtccgggtcgctgagcgctgcaagacaggggagggagccgggcgggagagggaggggcggcgccggggcgggccctgatatagagcaggcgccgcgggtcgcagcacagtgcggagaccgcagccccggagcccgggccagggtccacctgtccccgcagcgccggctcgcgccctcctgccgcagccaccggtgagtgccgcggtcctgagatccccgggccggatgcgcggcggccccagctcccgagcgtctgcctcccccgccctgggctgcccgggctccctgggctccccggcggctgcacggagtcaaggcgccccgtcccgggcgtcccccgcgggtgccgatccaggctgcccggagtccggagcccagagaggagagagacagctggggagcctggtcaccgcgggcatctcccctgcgctgcagtcgcccgcctggcctgccttcccgttcctccgcctcttgccctgacttctccttcctttgcagagccgccgtctagcgccccgacctcgccaccatgagagccctgctggcgcgcctgcttctctgcgtcctggtcgtgagcgactccaaagtgagtgcgctcttgctttgactgatgctgcccaaggacctctgatcagcaccaggggagaggaggggctgctcagggagctggggtcctccggattccatccacagcagggccagactctccccaggaaatgggacagggtggcagcggaggcttgagaaccacgggggttggcactggctggcaagggaggaagaggccgccgggactgccccagcctgcgggcatctggtagatgaagcttgcttgggtcaatccatttctcctggctggaaacccatggtcttccatttgagaactagatacgaacagggtgaggcgagagggagagggaagagtgggttttgggattggggccagtttaccctcaccctggagtccctggagcatgggacctttgatgaagcctcctcccgaatctcttccagggcagcaatgaacttcatcaagttccatgtgagtatccacccctacaacagttggctgcacagacaagttgggaaggcttcaggggacatcccctccctgccctctgctgcagggctgcgccaccccttaccacttccactccccctcgcttaccccacctttgttctctccagcgaactgtgactgtctaaatggaggaacatgtgtgtccaacaagtacttctccaacattcactggtgcaactgcccaaagaaattcggagggcagcactgtgaaataggtatggggatctccactgcaactgggagagaaatttggggacagggagggatgggtgggaggcaagagcaggcaggagttaggagctggaggtagggtgggtgacatcttcatccctatgtgacaagcataaacacacacacacgctcacgaaacagtggccacacaaatgtgaggtggggttggaaggagaccctgtccagtcttctggcaggtctgaaacgacatctttaaaatgtccgttggcagccgggcatggtggctcacgcttgtaatcccagcattttgagaggtcaaggttgagtggatcatttgaggtcaggagttcaagaccagcctggacaacatggtgtaaccctgcctctactaaaaatgcaaaaatcagcctggcatggtggtggatgcctgtagtcccagctacttgggaggctgaggcaggagaattgcttgaacctgggaggcagagatctcagtgagctgagatcacaccactgcactccaactgggcgacagagcaagactccatctcaaaaaaaaaaaataaaagttagttggaatgttcttctctttctcatattctctcatcctcctgtccccttgtagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggtgaggtgggggcaacaaggaccaaaagccctccctacagcttcccagaaaccttgttaccatccccttctcccagagggctggccatagcacaagagaagtgcggcctctggttgagtcttccctgaggggaggaggcagggaaggccctctgggttggaatgacatcccctatctttctgtgttgccaggaacccagacaaccggaggcgaccctggtgctatgtgcaggtgggcctaaagccgcttgtccaagagtgcatggtgcatgactgcgcagatggtgagcatcactgacctgctgatgacagtggggtggaaggggacaaacttacatgtccccttattccatcacaggaggactgaggaggtggggggtgcccgagagggatgctttctcctacctgcctccctaagacatccctctgtttgtcctccaggaaaaaagccctcctctcctccagaagaattaaaatttcagtgtggccaaaagactctgaggccccgctttaagattattgggggagaattcaccaccatcgagaaccagccctggtttgcggccatctacaggaggcaccgggggggctctgtcacctacgtgtgtggaggcagcctcatcagcccttgctgggtgatcagcgccacacactgcttcatgtacggccctgggtttctcctcttcgactcttctgccccaccccaagcacatccctttctccttcccagcaaagtgttccgcctcatttctccctcatctgcccctgtccatgcagcccatggccttggggacaagtcgtgctttgaggcctctagggagggaaggaagaagtggcagatttcatgggactaagctgtttgatgggtatcttcttccacagtgattacccaaagaaggaggactacatcgtctacctgggtcgctcaaggcttaactccaacacgcaaggggagatgaagtttgaggtggaaaacctcatcctacacaaggactacagcgctgacacgcttgctcaccacaacgacattggtgagggggaaccccgcgactactgtggccataatggcttggggagagtgggacccagggagagactggagctgaggttgaagctgcccggtggggcaggggtggggcgagggaccttgaagcctcgatatacatgacaaagggagtggcagggaagagttccatgaagtctgaggggcctggtgctcctctggagagaccctgaatttccccaacaagtagcctcttgcgagtggaaacagccctgtgggtatatggcttgggctgggaaggccctgtttatatgaattagaaaaagacacaccttcctttgtgggatgcagcctctgtctgtgctaggatatagaacttggagaatggagccttgggatggattccagcctaactacctcagctgggagtttttgcagaaacgacctgtacagctgtatgcagtggctctggccatccaagcctttttcaacacctggaacaaagcccttggggcatggggcaggggaggtttccaggtgataagcgaccagcagacctccctggatgactgacctagggataggcatagctacttcctcggcacttggaggggacagatggggaccgcctaaccagtagtgatctttctcctctgaccctctgtcctcccccagccttgctgaagatccgttccaaggagggcaggtgtgcgcagccatcccggactatacagaccatctgcctgccctcgatgtataacgatccccagtttggcacaagctgtgagatcactggctttggaaaagagaattctagtaagtgacaattgcgactgacttagaaggtcctgaggagtgttttgacctgaaaatgagcccagcgtgatcaagggaagactgcagagttagaggtgggagcactgaggcggtggcagatgggtccagggatggatgaagagtgttgtttagggagcgatgggctgcaaaggtaaatagatggtaggggctataggtggagtaaaggctcagatttgcatggaagagaataagggccttccctggtagagatactttatggttcccctctctggcagactcccagtggacagataaatcttgatgcaaacgcctccctgttttctccacctagccgactatctctatccggagcagctgaaaatgactgttgtgaagctgatttcccaccgggagtgtcagcagccccactactacggctctgaagtcaccaccaaaatgctgtgtgctgctgacccacagtggaaaacagattcctgccaggtgagtgttccaagcatctctctccacctcttccatatctccccagagctcctgggcttgttccagccagcttaagggtgtctctctctagccaaagccctaagtagccagaatcaggagctcaggtctttgagggtttaaaccagtccttatgtgtttgccagacattaccaaaaaaatcccagctctgcgctagtcacttcagactgggggcacgagatcctagaaagaggaaacagtaaaagacaatgtaactcagtgcccagggtgtgttgtgaactataaatgatcaggtgttcaggagagggaggtgagtgccaacctgagggtcagggaggggaggctttaaaggaaatgtgacttgataggcatttgaagaggcagagggaagaaaggaaggtgtttcagttgaaagatacaaaactgagaaggaggctggcatattccgggtggggaggagaactagggtctgggagtgtggatggaatagtggcagatgacagggcttttaaagccaagcaggggattttaaacttgatgtggtagaaaatggggctgcgtcaggcacagtggctcatgcctgtaatcccagcactttgggaggccgaggtggatggatcacttgaggccaggagtttgagaccggcctggccaacatggtgaaaccctgtgtctactaaaaatgcaaaaaaaaattagccaggtgtggtggtgcctgcctgtaatcccagctaatcaggaggctgagacatgggaatcgcttgagcacaggaggcaagtttgcagtgagctgagatcacgtcattgcacgccagcctgggcgacagagcgagattctgtcctccccccgaaaaaaagaaagaaaatgggaagtcgctaaggactttgactgggaaactcttccctctctctggtatggttgggtgatgggatcagaaatcccctcctcacttctctagggctcatcttttgtatctttggcgtcacagggagactcagggggacccctcgtctgttccctccaaggccgcatgactttgactggaattgtgagctggggccgtggatgtgccctgaaggacaagccaggcgtctacacgagagtctcacacttcttaccctggatccgcagtcacaccaaggaagagaatggcctggccctctgagggtccccagggaggaaacgggcaccacccgctttcttgctggttgtcatttttgcagtagagtcatctccatcagctgtaagaagagactgggaagataggctctgcacagatggatttgcctgtgccacccaccagggtgaacgacaatagctttaccctcaggcataggcctgggtgctggctgcccagacccctctggccaggatggaggggtggtcctgactcaacatgttactgaccagcaacttgtctttttctggactgaagcctgcaggagttaaaaagggcagggcatctcctgtgcatgggtgaagggagagccagctcccccgacggtgggcatttgtgaggcccatggttgagaaatgaataatttcccaattaggaagtgtaacagctgaggtctcttgagggagcttagccaatgtgggagcagcggtttggggagcagagacactaacgacttcagggcagggctctgatattccatgaatgtatcaggaaatatatatgtgtgtgtatgtttgcacacttgtgtgtgggctgtgagtgtaagtgtgagtaagagctggtgtctgattgttaagtctaaatatttccttaaactgtgtggactgtgatgccacacagagtggtctttctggagaggttataggtcactcctggggcctcttgggtcccccacgtgacagtgcctgggaatgtattattctgcagcatgacctgtgaccagcactgtctcagtttcactttcacatagatgtccctttcttggccagttatcccttccttttagcctagttcatccaatcctcactgggtggggtgaggaccactcctgtacactgaatatttatatttcactatttttatttatatttttgtaattttaaataaaagtgatcaataaaatgtgatttttctgatgacaaatctccctggtgcttgtatgggaaggagttggagtacataaaaaggagaaaataacaaaggtggactgcaccctagagtttcttatgggactgcatctctggacttaatggagctttgggaggtagaggttaggagagctgtagggcagggccaccacagcacttaatgtatacaaagtttccccaaaccacaatcccatggctgtgtaagtagtgtacaggcttagtgtcagctcccaattgcctccttaacctgatgcctttgtgcagtgcacatcttgtacagctgtacatggtggtcttctttagggttaactccacaacagtctcttcactctgcccccaacaatccttgagctgacctcaaccaagaagaacacctgtggccaggtgtagtggctcatgcctgtaatcctaacattttgagaggctgaggtgggagaatcgcttgagcccaggagtttgagaccagcctgggaaacttagtgagaccctgtctctgcaaaaaataaaaaaattagccaggtgtggtagcacatgtctgtagtcctagctacttgggaggctgaggcaggaggatcataagccaaggagttcaagtttacagtcagctgattgtgccactgcactccagcctaggtgacagagcaagaccctgtctcaaaaaaaaaaaaagaagaagaagaagaagaagacctgtaagtgaaaattgctggccaggctcagtggctcacgcctgtaatccagcactttgggaagccgaggtgtgtggatcacctgaggtcaggagtttgagaccagcctgaccaacatagtgaaaccccatctctactaaaaatacaaaaaaattagccgggtgtgggggtgggtgcctgtaatcccagctactcgggaggctgaggcaggagaatcatttgaacccgggaggcagaggttgcagtgagccgagatcgcaccactgcactccagcctgggcgacagagggaagacttcgcctcaaataaataaaaaaataaagaaagaaagaaaattgcttatccagaatgccagcttgactctgtggcattcaggaaacaaaaaacataatttcccatcatctgtgtgtgggactgatgctggaattttctcagtgtgttaacagagcttgtgaccagccattgctatgcctatgattagggggcccagaatctcaaagctggatgatgtcctaatgggggtgactatatattttgaacaaaacatcaagttctgaaaagctggggtgtagaaggcaggttgggagataattgggtatcaaaaattagaattgccaagctgtgttaatgggttggggcagtgtttcttaatcgagtcatctgaggaccttttaaaatatgcacacctacactccacctgtagagattgtgattcaggtcagggctggctccaggtctcgtggttttagaaaagctccctcagtgttactaacatgcaccactgattgacagcctttggggacaattggtcagaatatatgaagtgcccagaaaaattcaagatatggtcagcatgagttctctgcatgaacagccagttttctgagtggggtttggtggcacaaggtctcctggatttcctggttaaaggctttctggaccgtgcttccctagagggcaagaactccaagtatttggctttttcttttttttgaggcagggtctcactctgtcacccaggctggagtgcagtggcgcaatcttagctcactgcagcttctgcctcccaggttcaagcgattctcctgcctcagcctcccaagtagctgggattacaagcatgcactaccactacccggctcatttttgtgtttttggtagagatggggtttcaccctgttggccaggctggtctcgaactcccgacctcaggtgatctgcctccctt

(19) United States
(12) Patent Application Publication
(10) Pub. No.: US 2011/0038845 A1
(43) Pub. Date: Feb. 17, 2011

(54) Diagnosis and treatment for endometriosis

(76) Inventors: Mohamed Bedaiwy, Toronto (CA); Robert Casper, Toronto (CA)

Correspondence Address: HOWSON & HOWSON LLP, 501 OFFICE CENTER DRIVE, SUITE 210, FORT WASHINGTON, PA, 19034, US

(21) Appl. No.: 11/919,920

(22) PCT Filed: May 5, 2006
(86) PCT No.: PCT/CA2006/000716

§ 371 Date: Feb. 3, 2009

Related U.S. Application Data
(60) Provisional application No. 60/677,867, filed on May 5, 2005.
(60) Provisional application No. 60/779,808, filed on Mar. 7, 2006.

(51) Int. Cl. A61K 38/43; C12Q 1/68; A61P 15/00

(52) U.S. Cl. 424/94.1; 435/6

(57) ABSTRACT

The invention relates to detection of individuals having or at risk of developing endometriosis based on the presence of one or more polymorphism in a gene associated with the fibrinolytic pathway, and methods for treating or preventing endometriosis by modulating the fibrinolytic pathway.

### FIELD OF THE INVENTION

The invention relates to detection of individuals having or at risk of developing endometriosis based on the presence of one or more polymorphisms in one or more genes associated with the fibrinolytic pathway, and methods for treating or preventing endometriosis by modulating the fibrinolytic pathway.

### BACKGROUND OF THE INVENTION

Endometriosis is defined as the presence of endometrial-like tissue growing outside the uterine cavity. It is the commonest benign gynaecologic disorder in women of reproductive age. Endometriosis is the leading cause of hospitalization for gynaecologic surgery including hysterectomy [Ajossa S. et al.; Clin Exp Obstet Gynecol 1994; 21:195-197] and a major burden on the healthcare system [Zhao S Z et al.; Am J Manag Care 1998; 4:1127-1134]. It is also associated with significant impairment in quality of life for affected women due to severe pain during menstruation and sexual intercourse, and infertility.

The pathophysiology of endometriosis remains enigmatic. As a result, current therapeutic strategies are mainly palliative and non-curative. Surgery is the first-line treatment to remove ovarian endometriomas and to correct endometriosis-associated adhesions that can distort pelvic anatomy. Nevertheless, patients who undergo surgical procedures have recurrence of endometriosis in up to 47% of cases [Kettle L M and Hummel W P; Obstet Gynecol Clin North Am 1997; 24:361-373] and recurrence of adhesions in up to 89% of cases [Parker J D et al.; Fertil Steril 2005; 84:1457-1461]. For severe pelvic pain, medical therapy in the form of GnRH agonist administration with HT addback is probably most effective for long-term relief of symptoms and for rendering the endometriosis inactive [Mitwally M F and Casper R F; J. Assist. Reprod Genet 2002; 19: 384-389]. New research treatments include the use of aromatase inhibitors together with progestin [Ailawadi R K et al.; Fertil Steril; 81:290-296] or together with oral contraceptives [Amsterdam L L et al.; Fertil Steril 2005; 84:300-304]. However, endometriosis recurs once all these treatments are stopped [Donnex J et al.; Gynecol Obstet Invest 2002; 54: Suppl 1:2-7].

Retrograde menstruation is the most accepted theory for the development of endometriosis. Retrograde menstruation has been documented in 70-90% of normal reproductive age women [Koninckx, Br J Obstet Gynaecol. 1980 March; 87(3):177-83; Halme, Obstet Gynecol. 1984 August; 64(2):151-4; Liu, Br J Obstet Gynaecol. 1986 August; 93(8):859-62; Kruitwagen, Fertil Steril. 1991 February; 55(2):297-303; and Blumenkrantz, Obstet Gynecol. 1981 May; 57(5):667-70]. However, the prevalence of endometriosis is estimated to be 10-15% of reproductive age women. Consequently, the question of why only certain women who experience retrograde menstruation develop endometriosis while others do not remains unresolved.

### SUMMARY OF THE INVENTION

The present invention is based on the finding that the persistence of a fibrin matrix in peritoneal pockets as a result of hypofibrinolysis may allow menstrually deposited endometrial fragments to initiate endometriosis. In particular, the invention is based in part on the finding that inhibition of fibrinolysis by high PAI-1 activity results in persistence of fibrin matrix leading to the development of endometriosis in some women, while lower PAI-1 activity allows rapid fibrin clearance before endometrial fragments can invade and implant. Genetic variation in the fibrinolytic system can lead to persistence of fibrin matrix in peritoneal pockets following retrograde menstruation in women with the genetic variations, in particular women whose PAI-1 promotor contains the 4G allele, i.e. 4G/4G or 4G/5G genotypes.

Broadly stated the present invention relates to a method for treating endometriosis in a subject comprising reducing hypofibrinolysis in the subject. The invention also relates to a method for treating or preventing endometriosis in a subject comprising reducing fibrin matrix in peritoneal pockets in endometrium in the subject.

In an aspect, the invention provides a method of treating endometriosis in a subject comprising modulating the fibrinolytic pathway. In an aspect, the fibrinolytic pathway is modulated by modulating one or more of PAI-1, tissue plasminogen activator (tPA), urokinase plasminogen activator (uPA), and thrombin-activated fibinolysis inhibitor (TAFI).

In a particular aspect, PAI-1 is modulated by administering a PAI-1 drug. In an embodiment, the method comprises reducing or inhibiting PAI-1. PAI-1 may be reduced or inhibited by administering an antagonist of PAI-1.

In another particular aspect, TAFI is modulated by administering a TAFI drug. In an embodiment, the method comprises reducing or inhibiting TAFI. TAFI may be reduced or inhibited by administering an antagonist of TAFI.

In a further particular aspect, tPA is modulated by administering a tPA drug. In an embodiment, the method comprises increasing or enhancing tPA. tPA may be increased or enhanced by administering an agonist of tPA.

In a further particular aspect, uPA is modulated by administering an uPA drug. In an embodiment, the method comprises reducing or inhibiting uPA. uPA may be reduced or inhibited by administering an antagonist of uPA. In another embodiment, the method comprises increasing or enhancing uPA. uPA may be increased or enhanced by administering an agonist of uPA.

In another aspect, the invention provides a method of treatment or prophylaxis of endometriosis based on the presence of a polymorphism in a gene of the fibrinolytic pathway, in particular the polymorphism is a PAI-1 polymorphism, a TAFI polymorphism, a tPA polymorphism and/or an uPA polymorphism.

In a further aspect, the invention provides a method for treating endometriosis comprising obtaining a sample of biological material containing at least one nucleic acid from the subject; analyzing the nucleic acid to detect the presence of at least one polymorphism in a gene of the fibrinolytic pathway associated with endometriosis (e.g., PAI-1 gene, TAFI gene, tPA gene, and/or uPA gene); and treating the subject in such a way as to counteract the effect of any such polymorphism detected.

In a still further aspect of the invention, a method is provided for the prophylactic treatment of a subject with a genetic predisposition to endometriosis comprising obtaining a sample of biological material containing at least one nucleic acid from the subject; analyzing the nucleic acid to detect the presence of at least one polymorphism in a gene of the fibrinolytic pathway associated with endometriosis or a delay in fibrinolysis; and treating the subject.

The invention provides methods, reagents and kits for detecting an individual's increased or decreased risk for endometriosis and related diseases.

The invention provides a method of analyzing a nucleic acid from an individual to determine which nucleotides are present at polymorphic sites within a gene of the fibrinolytic pathway, in particular a PAI-1 gene, tPA gene, uPA gene, and/or TAFI gene, for example, to identify PAI-1, tPA, uPA and/or TAFI polymorphisms. The analysis can be performed on a plurality of individuals who are tested for the presence of the disease phenotype. The presence or absence of a disease phenotype or propensity for developing a disease state can then be correlated with a base or set of bases present at the polymorphic sites in the individual tested. Alternatively, this determination step is performed in such a way as to determine the identity of the polymorphisms.

The invention relates to methods for using polymorphisms associated with a gene of the fibrinolytic pathway, in particular a PAI-1 gene, TAFI gene, tPA gene, and/or uPA gene; to diagnose endometriosis.

In aspects of the invention, the methods are used to determine an individual's risk for endometriosis and related diseases. In the methods, the presence of at least one endometriosis-associated polymorphism, in particular a polymorphism in a gene of the fibrinolytic pathway, more particularly a PAI-1 gene, tPA gene, uPA gene, and/or TAFI gene, in a nucleic acid sample of the individual is detected. The presence of at least one, two or more polymorphisms provides an indication of the individual's risk for endometriosis. The individual's risk for endometriosis can be, e.g., either an increased risk or a decreased risk as compared to an individual without the at least one, two or more polymorphisms (e.g., an individual with a different allele at that polymorphic site). Accordingly, the at least one, two or more polymorphism can comprise a predisposing or a protective polymorphism in the PAI-1 gene, tPA gene, uPA gene, and/or TAFI gene, in particular the PAI-1 gene.

In particular, the invention provides a method for identifying a polymorphism in a PAI-1 gene, tPA gene, uPA gene, and/or TAFI gene, more particularly a PAI-1 gene and/or TAFI gene, that correlates with endometriosis. The method may comprise obtaining PAI-1 gene, tPA gene, uPA gene, and/or TAFI gene sequence information from a group of patients with endometriosis, identifying a site of at least one polymorphism in the PAI-1 gene, tPA gene, uPA gene, and/or TAFI gene, and determining genotypes at the site for individual patients in the group. The genotypes may be correlated with the disease severity, prognosis of the patient, or treatments. In particular, the method is performed on a sufficient population size to obtain a statistically significant correlation.

In an aspect the invention provides a method for diagnosing or aiding in the diagnosis of endometriosis in a subject comprising the steps of determining the genetic profile of genes of a fibrinolytic pathway of the subject, in particular the genetic profile of PAI-1, tPA, uPA, and/or TAFI genes, thereby diagnosing or aiding in the diagnosis of endometriosis.

In an aspect the invention provides a method for diagnosing a genetic susceptibility for endometriosis in a subject comprising obtaining a biological sample containing nucleic acids from the subject, and analyzing the nucleic acids to detect the presence or absence of one or more polymorphisms in one or more genes of a fibrinolytic pathway of the subject, in particular a PAI-1 gene, tPA gene, uPA gene, and/or TAFI gene, more particularly a PAI-1 gene and/or a TAFI gene, wherein the polymorphisms are associated with a genetic predisposition for endometriosis.

In an aspect, the invention provides a method for diagnosis of endometriosis in a subject having, or at risk of developing endometriosis comprising determining a genotype for the subject including one or more polymorphism sites in a gene of a fibrinolytic pathway, in particular a PAI-1 gene, tPA gene, uPA gene, and/or TAFI gene, more particularly a PAI-1 gene and/or TAFI gene.

In a particular aspect the invention provides a method for diagnosis of stage III or IV endometriosis in a subject comprising determining a genotype for the subject for one or more polymorphism sites in a gene of a fibrinolytic pathway, in particular a PAI-1 gene, tPA gene, uPA gene, and/or TAFI gene, more particularly a PAI-1 gene.

In an embodiment of the invention the polymorphism is a 4G polymorphism in the promoter region of the PAI-1 gene. The presence of a 4G polymorphism may be indicative of an increased likelihood of a subject having stage III or IV endometriosis.

In an aspect of the invention, a method is provided for the diagnosis of endometriosis comprising: (a) obtaining sample nucleic acid from an individual; (b) detecting the presence or absence of a variant nucleotide at position 837 of a PAI-1 gene; and (c) determining the status of the individual by reference to the polymorphism in the PAI-1 gene.

In an embodiment, the method for diagnosis is one in which the single nucleotide polymorphism at position 837 is the absence of a G.

In an aspect the invention provides a method of analyzing a nucleic acid comprising obtaining a nucleic acid from a subject and determining the base occupying position 837 of SEQ ID NO. 1.

In another aspect the invention provides a method for the diagnosis of a single nucleotide polymorphism in a PAI-1 gene in a human comprising determining the sequence of the nucleic acid of the human at position 837 (according to the GenBank Accession No. AF386492 and SEQ ID NO. 1), and determining the status of the human by reference to a polymorphism in the PAI-gene.

In another aspect, the invention provides a method for diagnosis of endometriosis in a subject having, or at risk of developing, endometriosis comprising determining for the subject the genotype of one or more polymorphism site in the PAI-1 gene corresponding to position 837 of SEQ ID NO. 1. The method may further comprise comparing the genotype with known genotypes which are indicative of endometriosis.

The present invention therefore provides a method of diagnosing endometriosis or determining the presence or absence of a PAI-1 haplotype in a subject by obtaining material from the subject comprising nucleic acids comprising one or more nucleotides at about position 837 of SEQ ID NO. 1 and determining the PAI-1 gene haplotype.

In a further aspect, the invention provides a method for diagnosis of a subject having or at risk of developing endometriosis comprising determining a genotype including a polymorphism site in the PAI-1 gene for the subject, wherein the polymorphism is defined by position 837 of SEQ ID NO. 1 or a polymorphism site linked thereto.

Genotyping may be determined at a combination of multiple polymorphism sites within the promoter region or outside the promoter region of a PAI-1 gene, or another gene of the fibrinolytic pathway.

In an embodiment of the invention the polymorphism is a substitution polymorphism in the coding region of the TAFI gene.

In an aspect of the invention, a method is provided for the diagnosis of endometriosis comprising: (a) obtaining sample nucleic acid from an individual; (b) detecting the presence or absence of a variant nucleotide at the position encoding amino acid 325 of TAFI; and (c) determining the status of the individual by reference to the polymorphism in the TAFI gene.

In an embodiment, the method for diagnosis is one in which there is a Thr325Ile mutation in TAFI (e.g., C to T substitution at position 1064 of SEQ ID NO. 9 or GenBank Accession No. NM_{—}001872).

In an aspect the invention provides a method of analyzing a nucleic acid comprising obtaining a nucleic acid from a subject and determining the base occupying the position encoding amino acid 325 of TAFI (e.g. position 1064 of SEQ ID NO. 9).

In another aspect the invention provides a method for the diagnosis of a single nucleotide polymorphism in a TAFI gene in a human comprising determining the sequence of the nucleic acid of the human at the position encoding amino acid 325 of TAFI position (e.g., position 1064 according to GenBank Accession No. NP_{—}0001872 and SEQ ID NO. 9), and determining the status of the human by reference to a polymorphism in the TAFI-gene.

In another aspect, the invention provides a method for diagnosis of endometriosis in a subject having, or at risk of developing endometriosis comprising determining for the subject the genotype of one or more polymorphism site in the TAFI-1 gene corresponding to the position encoding amino acid 325 of TAFI (e.g. position 1064 of SEQ ID NO. 9). The method may further comprise comparing the genotype with known genotypes which are indicative of endometriosis.

The present invention therefore provides a method of diagnosing endometriosis or determining the presence or absence of a TAFI haplotype in a subject by obtaining material from the subject comprising nucleic acids comprising one or more nucleotides at about position 1064 of SEQ ID NO. 9, and determining the TAFI gene haplotype.

In a further aspect, the invention provides a method for diagnosis of a subject having or at risk of developing endometriosis comprising determining a genotype including a polymorphism site in the TAFI gene for the subject, wherein the polymorphism is defined by position 1064 of SEQ ID NO. 9, or a polymorphism site linked thereto.

Genotyping may be determined at a combination of multiple polymorphism sites within the coding region or outside the coding region of a TAFI gene, or another gene of the fibrinolytic pathway.

In an embodiment of the invention the polymorphism is a substitution polymorphism in the tPA gene, in particular the enhancer region of the tPA gene.

In an aspect of the invention, a method is provided for the diagnosis of endometriosis comprising: (a) obtaining sample nucleic acid from an individual; (b) detecting the presence or absence of a variant nucleotide at position −7351 of a tPA gene enhancer sequence; and (c) determining the status of the individual by reference to the polymorphism in the tPA gene.

In an embodiment, the method for diagnosis is one in which there is a −7351C/T mutation.

In an aspect the invention provides a method of analyzing a nucleic acid comprising obtaining a nucleic acid from a subject and determining the base occupying position 7351 of SEQ ID NO. 23.

In another aspect the invention provides a method for the diagnosis of a single nucleotide polymorphism in a tPA gene in a human comprising determining the sequence of the nucleic acid of the human at position −7351 (e.g., according to the GenBank Accession No. Z48484 and SEQ ID NO. 23), and determining the status of the human by reference to a polymorphism in the tPA gene.

In another aspect, the invention provides a method for diagnosis of endometriosis in a subject having, or at risk of developing endometriosis comprising determining for the subject the genotype of one or more polymorphism site in the tPA gene corresponding to position −7351 of SEQ ID NO. 23. The method may further comprise comparing the genotype with known genotypes which are indicative of endometriosis.

The present invention therefore provides a method of diagnosing endometriosis or determining the presence or absence of a tPA haplotype in a subject by obtaining material from the subject comprising nucleic acids comprising one or more nucleotides at about position −7351 of SEQ ID NO. 23, and determining the tPA gene haplotype.

In a further aspect, the invention provides a method for diagnosis of a subject having or at risk of developing endometriosis comprising determining a genotype including a polymorphism site in the tPA gene for the subject, wherein the polymorphism is defined by position −7351 of SEQ ID NO. 23, or a polymorphism site linked thereto.

Genotyping may be determined at a combination of multiple polymorphism sites within the enhancer region or outside the enhancer region of a tPA gene, or another gene of the fibrinolytic pathway.

In an embodiment of the invention the polymorphism is a substitution polymorphism in the uPA gene. In a particular embodiment the polymorphism is a substitution in exon 6 encoding the kringle domain of the uPA gene. In another particular embodiment the polymorphism is a substitution in intron 7 of the uPA gene.

In an aspect, of the invention, a method is provided for the diagnosis of endometriosis comprising: (a) obtaining sample nucleic acid from an individual; (b) detecting the presence or absence of a variant nucleotide in exon 6 of the kringle domain or intron 7 of a uPA gene; and (c) determining the status of the individual by reference to the polymorphism in the uPA gene.

In an embodiment, the method for diagnosis is one in which there is a C→T polymorphism in exon 6.

In an embodiment, the method for diagnosis is one in which there is a T→C polymorphism in intron 7.

In an aspect the invention provides a method of analyzing a nucleic acid comprising obtaining a nucleic acid from an individual and determining the base occupying positions 3007 and/or 3637 of SEQ ID NO. 24.

In another aspect the invention provides a method for the diagnosis of a single nucleotide polymorphism in a uPA gene in a human comprising determining the sequence of the nucleic acid of the human at positions 3007 and/or 3637 (e.g., according to the GenBank Accession No. AF377330 and SEQ ID NO. 24), and determining the status of the human by reference to a polymorphism in the uPA gene.

In another aspect, the invention provides a method for diagnosis of endometriosis in a subject having, or at risk of developing endometriosis comprising determining for the subject the genotype of one or more polymorphism site in the uPA gene corresponding to a C→T polymorphism in exon 6 encoding the kringle domain or a T→C polymorphism in intron 7 [e.g., positions 3007 and 3637 of SEQ ID NO. 24, respectively]. The method may further comprise comparing the genotype with known genotypes which are indicative of endometriosis.

The present invention therefore provides a method of diagnosing endometriosis or determining the presence or absence of an uPA haplotype in a subject by obtaining material from the subject comprising nucleic acids comprising one or more nucleotides at about position 3007 and/or 3637 of SEQ ID NO. 24, and determining the uPA gene haplotype.

In a further aspect, the invention provides a method for diagnosis of a subject having or at risk of developing endometriosis comprising determining a genotype including a polymorphism site in the uPA gene for the subject, wherein the polymorphism is defined by position 3007 or 3637 of SEQ ID NO. 24, or a polymorphism site linked thereto.

Genotyping may be determined at a combination of multiple polymorphism sites within exon 6 or intron 7 or outside these regions of an uPA gene, or another gene of the fibrinolytic pathway.

In particular aspects, the presence of a polymorphism inherited from one of an individual's parents provides an indication of the individual's risk for endometriosis. In other aspects, the presence of the polymorphism inherited from both of the individual's parents provides an indication of the individual's risk for endometriosis.

In aspects of the invention methods for determining an individual's risk for endometriosis are provided comprising determining an individual's genotype at one or more polymorphic sites in a gene of the fibrinolytic pathway, in particular a PAI-1 gene, tPA gene, uPA gene, and/or TAFI gene, more particularly a PAI-1 gene and/or TAFI gene. A first genotype at the one or more polymorphic sites (e.g. PAI-1 polymorphism in particular 4G/4G) is statistically associated with an increased risk for endometriosis as compared to a second genotype at the polymorphic site (5G/5G PAI-1 genotype). Thus, for example, if the individual's genotype corresponds to the first genotype, the individual's risk for endometriosis is greater than that of other individuals who have the second genotype.

In an embodiment, a method is provided for determining an individual's risk for endometriosis comprising determining an individual's genotype at a promoter polymorphic site in a PAI-1 gene wherein an individual with a 4G/5G genotype has an about 10 to 20 times, more particularly 15 to 18 times, higher risk to have endometriosis than individual's with a 5G/5G genotype

In an embodiment, a method is provided for determining an individual's risk for endometriosis comprising determining an individual's genotype at a promoter polymorphic site in a PAI-1 gene wherein an individual with a 4G/4G genotype has an about 50 to 150, 100 to 150, 100 to 130, 115 to 130, 120 to 130, or 125 times higher risk to have endometriosis than individual's with a 5G/5G genotype.

In some aspects, the presence of a single allele of a particular polymorphism (e.g. PAI-1 polymorphism, in particular 4G) is sufficient to indicate whether the individual's risk of endometriosis is increased or decreased. In other aspects, two copies of an allele of a particular polymorphism (e.g. PAI-1 polymorphism, in particular 4G/4G) must be present to indicate an increased or decreased risk of endometriosis. Determining the individual's genotype typically involves obtaining a nucleic acid sample from the individual, and determining the individual's genotype by amplifying at least a portion of a gene of the fibrinolytic pathway (e.g PAI-1 gene, TAFI gene, tPA gene and/or uPA gene) from the sample, the portion comprising one or more polymorphic sites. Such amplification directly determines the genotype or facilitates detection of one or more polymorphisms by an additional step. In one aspect, the individual's genotype is determined by performing an allele-specific amplification or an allele-specific extension reaction. In another aspect, the individual's genotype is determined by sequencing at least a portion of the gene from the sample, the portion comprising at least one polymorphic site. In yet another aspect, the individual's genotype is determined by hybridization of a nucleic acid probe, optionally after amplification of at least a portion of the gene. In particular aspects, at least one polymorphic site consists of a single nucleotide position, and the sample is contacted with at least one sequence-specific oligonucleotide probe under stringent conditions. In an aspect, the probe hybridizes under stringent conditions to nucleic acids in the sample when a first nucleotide does not occupy the nucleotide position defining the polymorphic site but not when the first nucleotide occupies the nucleotide position. Hybridization of the probe to the nucleic acid sample is detected using methods known in the art.

Diagnostic methods of the invention are optionally combined with known clinical methods to diagnose endometriosis. Thus, the methods optionally include performing at least one clinical test for endometriosis.

The invention provides a method for screening subjects comprising obtaining sequence information for one or more genes of the fibrinolytic pathway, in particular PAI-1 gene, tPA gene, uPA gene, and/or TAFI gene sequence information, in particular PAI-1 and/or TAFI gene sequence information, from the subject and determining the identity of one or more polymorphisms in the gene(s) that is indicative of endometriosis. The subject may be at risk of developing endometriosis or have endometriosis.

The invention also provides a method for determining the efficacy of a treatment for a particular subject with endometriosis based on genotype comprising (a) determining the genotype for one or more polymorphism sites in a gene of the fibrinolytic pathway, in particular a PAI-1 gene, tPA gene, uPA gene, and/or TAFI gene, more particularly a PAI-1 gene and/or TAFI gene, for a group of subjects receiving a treatment; (b) sorting subjects into subgroups based on their genotype; (c) identifying correlations between the subgroups and the efficacy of the treatment in the subjects, (d) determining the genotype for the same polymorphism sites in the gene(s) of the particular subject and determining the efficacy of the treatment for the particular subject based on a comparison of the genotype with the correlations identified in (c).

The invention further provides a method for classifying a subject who is or is not at risk for developing endometriosis as a candidate for a particular course of therapy or a particular diagnostic evaluation.

The invention still further provides a method for selecting a clinical course of therapy or a diagnostic evaluation to treat a subject who is or is not at risk for developing endometriosis.

The invention also relates to a kit for determining the presence of a polymorphism disclosed herein, in particular a PAI-1 polymorphism, tPA polymorphism, uPA polymorphism, and/or TAFI polymorphism.

An aspect of the invention provides kits for detecting the presence of one or more predisposing or protective polymorphisms in one or more genes of the fibrinolytic pathway, in particular a PAI-1 gene, tPA gene, uPA gene, and/or TAFI gene, more particularly a PAI-1 gene and/or TAFI gene, e.g., in a nucleic acid sample of a subject whose risk for endometriosis is being assessed. In an aspect, the invention provides a kit including one or more oligonucleotides capable of detecting one or more polymorphisms in one or more genes of the fibrinolytic pathway and instructions for detecting the polymorphisms with the oligonucleotides and for correlating the detection to the subject's risk for endometriosis, packaged in one or more containers. Essentially all of the features noted for the method embodiments above apply to the kit embodiment.

In another aspect, a kit comprises in a package a restriction enzyme capable of distinguishing alternate nucleotides at the polymorphism site or a labeled oligonucleotide being sufficiently complementary to the polymorphism site and capable of distinguishing the alternate nucleotides at the polymorphism site (i.e. probes). A kit may also comprise primers to amplify a region surrounding the polymorphism site, a polymerization agent and instructions for using the kit to determine genotype.

In a further aspect, the invention provides a kit for the detection of a polymorphism comprising at a minimum, at least one polynucleotide of at least 10 contiguous nucleotides of a gene of the fibrinolytic pathway, in particular a PAI-1 gene, tPA gene, uPA gene, and/or TAFI gene, more particularly a PAI-1 gene (e.g. SEQ ID NO. 1) and/or TAFI gene (e.g. SEQ ID NO. 9), or their complements, wherein at least one polynucleotide contains at least one polymorphic sited associated with endometriosis or a delay in fibrinolysis.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1 shows the agarose gel electrophoresis (1.5%) of PCR-amplified fragments from the PAI-1 promotor region, showing the 400 bp product that includes the 4G/5G polymorphic site from the 1^{st} to the 6^{th} lane (from the left). The 7^{th} lane (from the left) shows the negative control. The gel was stained with ethidium bromide to permit visualization of DNA bands upon ultraviolet irradiation

FIG. 2 shows the plasminogen activator inhibitor-1 (PAI-1) gene polymorphism of 8 patients. The amplified DNA of 4G polymerase chain reaction using the (Deletion) primers is shown in the upper panel and that of the 50 using the (Insertion) primers in the lower panel. Patients of 1, 2, and 3 are homozygous for 4G/4G. Patients 4, 5 and 6 are homozygous for 5G/5G. Patients 7 and 8 are heterozygous 4G/5G.

FIG. 3 is a schematic diagram of the coagulation and fibrinolytic pathways.

FIG. 4 is a schematic diagram of the activation and inhibition of the fibrinolytic pathway.

FIG. 5 shows immunoblots of nonspecific and allele specific PCR. Panel A. Non-specific PCR: the agarose gel electrophoresis (1.5%) of PCR-amplified fragments from the PAI-1 promotor region, showing the 400 bp product that includes the polymorphic site of the promoter region of the PAI-1 gene from the 2^{nd} to the 5^{th} lane (from the left). The 1^{st} lane (from the left) shows the negative control. The gel was stained with ethidium bromide to permit visualization of DNA bands upon ultraviolet irradiation. Panel B: Allele specific PCR: plasminogen activator inhibitor-1 (PAI-1) genotype of 5 patients showing the amplified DNA of the 4G polymerase chain reaction using the deletion primers (upper part of the panel) and that of the 50 using the insertion primers (lower part of the panel). Patients 1&2 are homozygous for 4G/4G. Patients 3&4 are heterozygous 4G/5G. Patient 5 is homozygous for 5G/5G.

FIG. 6: Proposed interaction between the plasminogen activator inhibitor-1 (PAI-1) genotype and retrograde menstruation in the initiation of endometriosis. Plasminogen-activator inhibitor type 1 (PAI-1) 4G allele, particularly the homozygous form (4G/4G), is associated with hypofibrinolysis leading to persistence of fibrin matrix in the peritoneal cavity at the time of retrograde menstruation (2b). The fibrin clot provides the surface on which the retrograde menstrual constituents, including endometrial fragments, starts to invade and implant (3b) leading to the initiation of endometriosis (4b). On the other side, individuals with efficient fibrinolysis, namely those with 5G/5G genotype (2a), fail to form enough fibrin matrix to support the growth and invasion of trapped endometrial fragments deposited by retrograde menstruation (3b). The 5G/5G genotype is associated with rapid clearance of all retrograde menstrual constituents and failure of initiation of endometriosis (4a).

### DEFINITIONS

In accordance with the present invention there may be employed conventional techniques of molecular biology and nucleic acid chemistry within the skill of the art. Such techniques are explained fully in the literature. See for example, Sambrook et al, Molecular Cloning: A Laboratory Manual, Third Edition (2001) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Oligonucleotide Synthesis (M. J. Gait, ed., 1984); the series, Methods in Enzymology (Academic Press, Inc.); the series Current Protocols in Human Genetics (Dracopoli et al., eds., 1984 with quarterly updates, John Wiley & Sons, Inc.); Berger and Kimmel, Guide to Molecular Cloning Techniques, Methods in Enzymology volume 152 Academic Press, Inc., San Diego, Calif.; Current Protocols in Molecular Biology, F. M. Ausubel et al., eds., Current Protocols, a joint venture between Greene Publishing Associates, Inc. and John Wiley & Sons, Inc., (supplemented through 2004); Freshney (1994) Culture of Animal Cells, a Manual of Basic Technique, third edition, Wiley-Liss, New York and the references cited therein; Fundamental Methods Springer Lab Manual, Springer-Verlag (Berlin Heidelberg New York); and Atlas and Parks (Eds.), The Handbook of Microbiological Media (1993) CRC Press, Boca Raton, Fla., all of which are incorporated herein by reference.

Unless defined otherwise, all technical and scientific terms used herein have the same meaning as commonly understood by one of ordinary skill in the art to which this invention belongs. The following definitions supplement those in the art and are directed to the present application and are not to be imputed to any related or unrelated case. Although any methods and materials similar or equivalent to those described herein can be used in the practice for testing of the invention, particular materials and methods are described herein.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made.

The terms “sample”, “biological sample”, and the like mean a material known or suspected of expressing or containing a nucleic acid comprising a polymorphism associated with endometriosis, or one or more genes of the fibrinolytic pathway, in particular a PAI-1 gene, tPA gene, uPA gene, and/or TAFI gene. In aspects of the invention, the sample contains a PAI-1 gene or part thereof. The test sample can be used directly as obtained from the source or following a pretreatment to modify the character of the sample. The sample can be derived from any biological source, such as tissues (e.g. endometrial tissue), extracts, cells, or cell cultures, including nucleated cells, cell lysates, conditioned medium from fetal or maternal cells, and physiological fluids, such as, for example, whole blood, plasma, serum, saliva, ocular lens fluid, cerebral spinal fluid, sweat, urine, milk, ascites fluid, amniotic fluid, vaginal fluid, synovial fluid, peritoneal fluid, and the like. In aspects of the invention the sample is blood.

A sample can be treated prior to use, such as preparing plasma from blood, diluting viscous fluids, and the like. Methods of treatment can involve filtration, distillation, extraction, concentration, inactivation of interfering components, the addition of reagents, and the like. Nucleic acids may be isolated from the samples and utilized in the methods of the invention. Thus, a test sample may be a nucleic acid sequence corresponding to the sequence in the test sample, that is all or part of the region in the sample nucleic acid may first be amplified using a conventional technique such as PCR, before being analyzed for sequence variation. A nucleic acid sample may comprise RNA, mRNA, DNA, cDNA, genomic DNA, and oligonucleotides, and may be double-stranded or single-stranded. The nucleic acids may be sense strands, the non-coding regions, and/or the antisense strands, and can include all or a portion of the coding sequence of a gene, or additionally or alternatively non-coding regions such as introns, and non-coding sequences including regulatory sequences (e.g. a promoter, enhancer). A nucleic acid can be fused to a marker sequence, for example, a sequence that is used to purify the nucleic acid. In embodiments of the invention the sample is a mammalian sample, preferably human sample.

In another embodiment the sample is a physiological fluid, in particular blood. In particular embodiments a sample comprises nucleic acids or DNA obtained from nucleated cells in blood, from cells obtained by buccal smear or cheek swab, or from any other source of nucleated cells in the body.

The terms “subject”, “individual”, and “patient” refer to an animal including a warm-blooded animal such as a mammal, which is afflicted with or suspected of having or being pre-disposed to, or at risk of developing endometriosis. Mammal includes without limitation any members of the Mammalia. In general, the terms refer to a human. The terms also include animals bred for food, pets, or sports, including domestic animals such as horses, cows, sheep, poultry, fish, pigs, and goats, and cats, dogs, and zoo animals, apes (e.g. gorilla or chimpanzee), and rodents such as rats and mice. The methods herein for use on subjects/individuals/patients contemplate prophylactic as well as curative use. Typical subjects for treatment include persons susceptible to, suffering from or that have suffered endometriosis or a related disease. In certain aspects, the subject is a woman undergoing diagnostic laparoscopy for pelvic pain. In certain other aspects, the subject is a prepubertal girl and methods described herein are employed to determine predisposition to developing endometriosis post puberty.

“Modulate” and its various grammatical variations, in aspects of the invention, refer to either the enhancement, inhibition, or suppression of a gene of the fibrinolytic pathway (e.g., PAI-1, uPA, tPA, and/or TAFI).

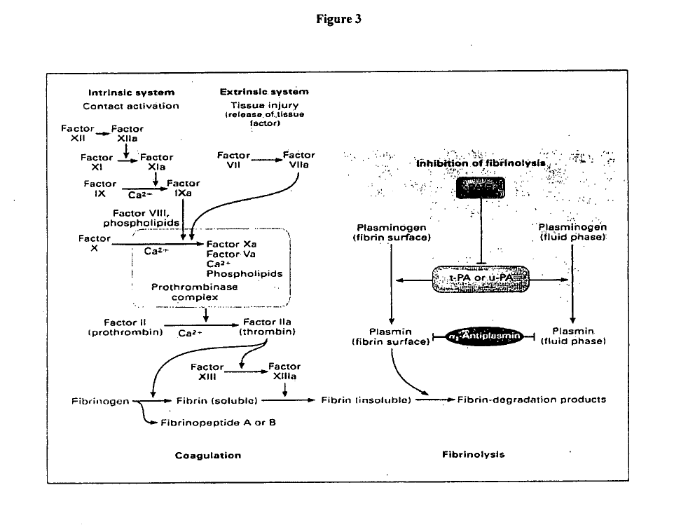

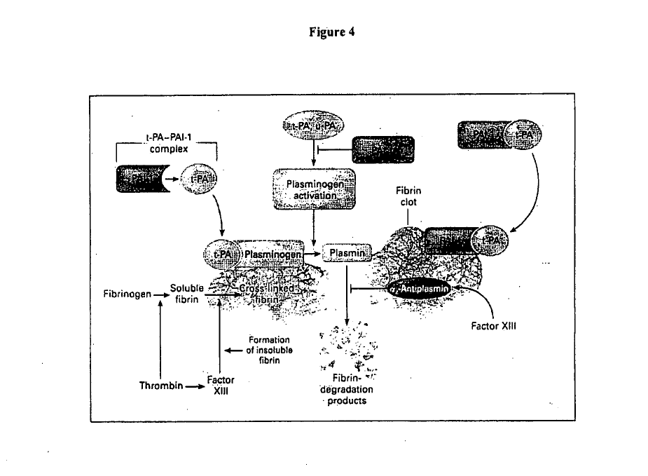

“Fibrinolytic pathway” refers to the pathway by which thrombin-generated fibrin clots are removed. The major components of the fibrinolytic pathway are plasminogen, plasminogen activators and inhibitors, and plasmin inhibitors. Plasminogen is the proenzyme in plasma, which upon conversion to its active form, plasmin, is considered primarily responsible for the digestion of fibrin clots. FIG. 3 is a schematic diagram of the coagulation and fibrinolytic pathway, and the activation and inhibition of the fibrinolytic pathway is shown in FIG. 4.

The terms “polynucleotide” and “oligonucleotide” refer to single-stranded or double-stranded nucleotide polymers comprised of more than two nucleotide subunits covalently joined together. The nucleotides may comprise ribonucleotides, deoxyribonucleotides, and/or any other N-glycoside of a purine or pyrimidine base, or modified purine or pyrimidine bases, non-standard or derivatized base moieties (see for example, U.S. Pat. Nos. 6,001,611, 5,955,589, 5,844,063, 5,789,562, 5,750,3343, 5,728,525, and 5,679,785), or any combination thereof. The sugar groups of the nucleotide subunits may also comprise modified derivatives of ribose or deoxyribose, (e.g. o-methyl ribose). Subunits may be joined by phosphodiester linkages, phosphorothioate linkages, methyl phosphonate linkages or by other linkages, including rare or non-naturally occurring linkages that do not interfere with hybridization. An oligonucleotide may have uncommon nucleotides or non-nucleotide subunits.

Oligonucleotides that are primer or probe sequences may comprise DNA, RNA, or nucleic acid analogs including uncharged nucleic acid analogs such as peptide nucleic acids (PNAs) (see PCT Published Application No. WO92/20702; Nielsen et al, Science 254, 1497-1500, 1991), morpholino analogs (see U.S. Pat. Nos. 5,185,444, 5,034,506, and 5,142,047), and N3′-P5′-phosphoamidate (PA) analogs (see for example, U.S. Pat. No. 6,169,170).

Polynucleotides and oligonucleotides may be prepared using methods known in the art, including synthetic, recombinant, ex vivo generation, or a combination thereof, as well as conventional purification methods. For example, polynucleotides and oligonucleotides can be synthesized using nucleotide phosphoramidite chemistry, in particular using instruments available from Applied Biosystems, Inc (Foster City, Calif.), DuPont (Wilmington, Det) or Milligen (Bedford, Mass.).

When desirable, polynucleotides and oligonucleotides may be labeled using methods known in the art (see U.S. Pat. Nos. 5,464,746; 5,424,414; and 4,948,882). Polynucleotides and oligonucleotides, including labeled or modified polynucleotides and oligonucleotides, can also be obtained from commercial sources. For example, polynucleotides and oligonucleotides can be ordered from QIAGEN (http://oligos.qiagen.com), The Midland Certified Reagent Company (www.mcrc.com), and ExpressGen Inc (Chicago, Ill.).

“PAI-1 gene” refers to a sequence encoding a mammalian plasminogen activator inhibitor-1 (PAI-1 or serine-cysteine proteinase inhibitor Glade E member 1 (Serpine1). PAI-1 is a linear glycoprotein that is composed of 379 amino acids and has a molecular weight of 48,000 KD [Kruithof E K; Enzyme 1988; 40:113-121]. It binds rapidly to tissue plasminogen activator (t-PA) and to urokinase plasminogen activator (u-PA) in a ratio of 1:1 [Lindahl T L et al.; Biochem J 1990; 265:109-113], forming a stable complex that is cleared from the circulation by hepatic cells [Owensby D A et al.; J Biol Chem 1991; 266:4334-4340]. The human PAI-1 gene is located on chromosome 7 and contains 9 exons and 8 introns [Strandberg et al., Eur J Biochem. 1988 Oct. 1; 176 (3):609-16.; Kohler et al, N Engl J Med. 2000 Jun. 15; 342(24):1792-801]. The active form of PAI-1, synthesized in platelets and endothelial cells, is unstable, with a half-life of 30 minutes [Kooistra, Biochem J. 1986 Nov. 1; 239 (3):497-503].

Sequences for the PAI-1 gene are found in GenBank under Gene ID. No. 5054 and Accession Nos. AF386492 and NM_{—}000602, and gene links therein. The term includes the coding region, non-coding region preceding (leader) and following coding regions, introns, and exons of a PAI-1 sequence. In particular, the PAI-1 gene includes the promoter. A “promoter” is a regulatory sequence of DNA that is involved in the binding of RNA polymerase to initiate transcription of a gene and is considered part of the corresponding gene.

“TAFI gene” refers to a sequence encoding a thrombin-activatable fibrinolysis inhibitor (TAFI or plasma procarboxypeptidase). An amino acid sequence for TAFI can be found in GenBank under Accession No. NP_{—}001863. Sequences for the TAFI gene are found in GenBank under Gene ID. No 1361 and Accession No. NP_{—}001782 and NM_{—}016413 and gene links therein. The term includes the coding region, non-coding region preceding (leader) and following coding regions, introns, and exons of a TAFI sequence. In particular, the TAFI gene includes the promoter or coding sequence.

“tPA gene” refers to a sequence encoding a tissue-type plasminogen activator. Sequences for the t-PA gene are found in GenBank under Gene ID. No. 5327 and gene links therein. The term includes the coding region, non-coding region preceding (leader) and following coding regions, introns, and exons of a tPA gene sequence. In particular, the t-PA gene comprises the enhancer sequence of Accession No. Z48484 (SEQ ID NO. 23).

“uPA gene” refers to a sequence encoding a urokinase-type plasminogen activator. The uPA molecule consists of two polypeptide chains linked by a disulphide bridge. The C-terminal sequence contains the serine protease domain, the N-terminal, growth factor-like domain, which may interact with polyanionic substrates such as proteoglycans [Estreicher A et al.; J Cell Biol 1990; 111:783-792]. Sequences for the t-PA gene are found in GenBank Accession Nos. X02419, AF377330, and NM_{—}002658, and under Gene ID. No. 5328 and gene links therein. The term includes the coding region, non-coding region preceding (leader) and following coding regions, introns, and exons of a uPA gene sequence. In particular, the uPA gene includes exon 6 encoding the kringle domain, and/or intron 7.

“Polymorphism” or “polymorphism site” refers to a set of genetic variants at a particular genetic locus among individuals in a population. A “single nucleotide polymorphism” (SNP) occurs at a polymorphic site occupied by a single nucleotide which is the site of variation between allelic sequences. The site is usually preceded by and followed by highly conserved sequences of the allele. In the context of the present invention the term refers to a set of genetic variants in genetic loci associated with the fibrinolytic pathway and with endometriosis, a predisposition to endometriosis, and/or reduced or delayed fibrinolysis. In aspects of the invention the term refers to genetic variants of a gene of the fibrinolytic pathway, in particular a PAI-1 gene, tPA gene, uPA gene, and/or TAFI gene, more particularly a PAI-1 gene.

In aspects of the invention, the polymorphism is a PAI-1 polymorphism. In particular aspects the polymorphism is within the promoter region of a PAI-1 gene, more particularly a single-base-pair polymorphism at position 675 bp upstream of the transcriptional start site of the PAI-1 promoter sequence [Dawson S J et al; Arterioscler Thromb 1991; 11:183-190]. This polymorphism induces two alleles containing either 4 or 5 sequential guanosines (G).

In an aspect the polymorphism corresponds to the region defined by position 837 of SEQ ID NO. 1 [for example, a 4G/5G (heterozygous polymorphism) or 4G/4G geneotype (homozygous polymorphism], and optionally flanking sequences thereto (e.g., SEQ ID NO. 2). The 4G allele is a single base pair deletion promoter polymorphism of the PAI-1 gene. The polymorphism may correspond to a site linked to a PAI-1 gene polymorphism, in particular a site linked to a polymorphism which corresponds to the region at position 837 of SEQ ID NO.1.

In aspects of the invention, the polymorphism is a TAFI polymorphism. In particular aspects the polymorphism is within the coding region of a TAFI gene. In an embodiment, the polymorphism is a Thr325Ile mutation in the TAFI sequence [Brouwers G J et al; Blood 2001; 98:1992-1993; Zhao L et al.; Thromb Haemost 1998; 80: 949-955]. A TAFI polymorphism at amino acid 325 may result from a C to T mutation at position 1064 of the TAFI gene sequence as shown in SEQ ID NO. 9 or at position 1040 of GenBank No. NM_{—}016413. The substitution results in the conversion of a Thr codon (ACU) to an Ile codon (AUU) at amino acid position 325. A TAFI polymorphism may be defined by position 1064, and optionally flanking sequences thereto, of a TAFI gene comprising SEQ ID NO.9. A TAFI polymorphism may be found in the GenBank SNP database as re1926447. A TAFI gene polymorphism may also be another polymorphism that affects TAFI Ag levels identified in Brouwers G F et al. [Blood 2001; 98:1992-1993]. The polymorphism may correspond to a site linked to a TAFI gene polymorphism, in particular a TAFI gene polymorphism which corresponds to the region encoding the amino acid at position 325 of TAFI (e.g. position 1064 of SEQ ID NO.9).

In aspects of the invention, the polymorphism is a tPA polymorphism. A tPA polymorphism may be a polymorphism identified by Ladenvall P et al [Ladenvall P et al.; Thromb Haemost 2002; 87:105-109]. In particular aspects the polymorphism is within the enhancer region of a tPA gene, more particularly a substitution at position −7351 of a tPA enhancer region. In an embodiment, the tPA polymorphism is a substitution of a thymidine (T) for cytosine (C) at position −7351 of a tPA gene [Ladenvall P et al.; Thromb Haemost 2002; 87:105-109; Ladenvall P et al; 2002; 87:105-109; SEQ ID NO. 23]. A tPA polymorphism may be defined by position −7351, and optionally flanking sequences thereto, of a tPA gene comprising SEQ ID NO. 23. The polymorphism may correspond to a site linked to a tPA polymorphism, in particular a site linked to a polymorphism which corresponds to the region at position −7351.

In aspects of the invention, the polymorphism is an uPA polymorphism. An uPA polymorphism may be a polymorphism identified by Conne B et al. [Thromb Haemost 1997; 77:433-435]. In particular aspects, the polymorphism is within the sequence encoding the kringle domain, more particularly a substitution from C to T in the sequence of exon 6 encoding the kringle domain (C→T polymorphism) that results in a Pro to Leu replacement. In an embodiment, the uPA polymorphism is a substitution of a cytosine (C) for thymidine (T) at position 3007 of SEQ ID NO. 24. In other particular aspects, the polymorphism is within intron 7, more particularly a T to C change in intron 7 (T→C polymorphism). In an embodiment, the uPA polymorphism is a substitution of a thymidine (T) for a cytosine (C) at position 3637 of SEQ ID NO. 24. An uPA polymorphism may be defined by position 3007, and optionally flanking sequences thereto, of an uPA gene comprising SEQ ID NO. 24. An uPA polymorphism may also be defined by position 3637, and optionally flanking sequences thereto, of an uPA gene comprising SEQ ID NO. 24. The polymorphism may correspond to a site linked to an uPA gene polymorphism, in particular a site linked to a polymorphism which corresponds to the region of a C→T polymorphism or T→C polymorphism.

Additional polymorphism sites may correspond to one or more polymorphisms within or outside the coding or regulatory region (e.g., promoter, enhancer) of a PAI-1 gene, tPA gene, uPA gene, and/or TAFI gene. In aspects of the invention the additional polymorphism sites are within the PAI-1 gene, including without limitation one or more of positions 664, 2037, 2362, 2852, 4588, 5404, 5686, 5834, 5878, 5984, 6821, 7729, 7343, 7365, 7771, 9759, 10381, 11312, 12750, and 13605 of a PAI-1 gene. Other polymorphism sites, including polymorphisms linked to these polymorphisms, in particular linked to the polymorphism at position 837 of SEQ ID NO. 1 and/or the polymorphism at the position encoding the amino acid at position 325 of TAFI (e.g. position 1064 of SEQ ID NO. 9], may be determined using conventional methods.

It will be noted that the numerical designations of the positions of the polymorphisms within a sequence are sequence specific and the same numerical positions may be assigned different numerical designations depending on the way in which the sequence is numbered and the sequence selected. In addition, sequence variations within a population, including insertions or deletions may change the relative position of the polymorphism and subsequently the numerical designations of particular nucleotides at and around a polymorphism.

The terms “trait” and “phenotype”, used interchangeably herein, refer to any visible, detectable, or otherwise measurable property of an organism such as symptoms of, or susceptibility to endometriosis. Generally, the terms are used herein to refer to symptoms, or susceptibility to endometriosis, or to an individual's response to an agent acting on endometriosis (e.g. PAI-1 drug and/or TAFI drug), or to symptoms of, or susceptibility to side effects to an agent acting on endometriosis.

The term “genotype” refers to the identity of alleles present in an individual or a sample. In the context of the present invention the term particularly refers to the description of the polymorphic alleles present in an individual or a sample. “Genotyping” a sample or an individual for a polymorphic marker involves determining the specific allele or the specific nucleotide carried by an individual at a polymorphic marker.

“Allele” refers to variants of a nucleotide sequence.

“Genetic variant” or “variant” refers to a specific genetic variant which is present at a particular genetic locus in at least one individual in a population and that differs from the wild type.

The term “haplotype” refers to the combination of alleles on one chromosome. In the context of the present invention it may refer to a combination of polymorphisms found in an individual which may be associated with a phenotype.

“Genetic predisposition”, “genetic susceptibility” and “susceptibility” all refer to the likelihood that a subject will develop a disease (e.g. endometriosis). A subject with an increased susceptibility or predisposition will be more likely than average to develop a disease while a subject with a decreased predisposition will be less likely than average to develop the disease. A genetic variant is associated with an altered susceptibility or predisposition if the allele frequency of the genetic variant in a population with a disease or disorder varies from its allele frequency in a control population without the disease or disorder or a wild type sequence by at least about 5%, 10%, 15%, 20%, 25%, 30%, 35%, 40%, 50%, 55%, 60% or 65%.

“Allele frequency” refers to the frequency that a given allele appears in a population.

The term “primer” refers to an oligonucleotide that has a hybridization specificity sufficient for the initiation of an enzymatic polymerization under predetermined conditions in an amplification reaction, a sequencing method, a reverse transcription method, and similar reactions and methods. For example, a primer can be a single-stranded oligonucleotide capable of acting as a point of initiation of template-directed DNA synthesis under suitable conditions (e.g., in the presence of four different nucleoside triphosphates and an agent for polymerization, such as DNA or RNA polymerase or reverse transcriptase) in an appropriate buffer and at a suitable temperature. The length of a primer will depend on its intended use but typically ranges from 15 to 30 nucleotides. A primer need not reflect the exact sequence of the template but it must be sufficiently complementary to hybridize with a template.

A “probe” refers to a nucleic acid capable of binding in a base-specific manner to a complementary strand of nucleic acid, such as a complementary strand of nucleic acid to be identified in a sample under predetermined conditions, for example in an amplification technique such as a 5′-nuclease reaction, a hybridization-dependent detection method (e.g. Southern or Northern blot), and the like.

Hybridizations with probes can be generally performed under “stringent conditions”. For example, hybridizations may be performed at a salt concentration of no more than 1M and a temperature of at least 25° C. In methods of the invention for allele-specific probe hybridizations, conditions of 5×SPPE (750 mM NaCL, 50 mM NaPhosphate, 5 mM EDTA, pH 7.4) and a temperature of 25-30° C. can be used. However, one skilled in the art could readily substitute other compositions of equal suitability.

A probe may be an immobilized probe i.e., a probe may be immobilized on a solid support by covalent bonding, absorption, hydrophobic and/or electrostatic interaction, direct synthesis on a solid support, and the like. The probes may be labeled with labels such as radioactive isotopes, enzymes, in particular enzymes capable of acting on a chromogenic, fluorescent or luminescent substrate (e.g. peroxidase or alkaline phosphatase), chromophoric chemical compounds, acridinium esters (see U.S. Pat. No. 5,185,439), substrates, cofactors, inhibitors, magnetic particles, chromogenic, fluorigenic or luminescent compounds, analogues of nucleotide bases, and ligands (e.g., biotin). Examples of fluorescent compounds include fluorescein, carboxyfluorescein, tetrachlorofluorescein, hexachlorofluorescein, Cy3, Cy3.5, Cy5, tetramethylrhodamine, rhodamine and its derivatives (e.g. carboxy-X-rhodamine), and Texas Red. Examples of luminescent compounds include luciferin, and 2,3-dihydrophthalazinediones (e.g. luminol). Examples of radioactive isotopes include ³H, ³⁵S, ³²P, ¹²⁵I, ⁵⁷Co, and ¹⁴C. Many labels are commercially available and can be used in the context of the present invention.

Probes and primers can be modified with chemical groups to enhance their performance or facilitate the characterization of hybridization or amplification products. In an aspect of the invention, the probes or primers have modified backbones (e.g., phosphorothioate or methylphosphonate groups) which render the oligonucleotides resistant to the nucleolytic activity of certain polymerases or to nucleases. Non-nucleotide linkers (e.g. EP No. 0313219) that do not interfere with hybridization or elongation of the primer can also be incorporated in the nucleic acid chain. A 3′ end of an amplification primer or probe may be blocked to prevent initiation of DNA synthesis (see WO 94/03472), or the 5′ end may be modified so that it is resistant to the 5′exonuclease activity present in some polymerases.

The term “sequencing” refers to a method for determining the order of nucleotides in a nucleic acid. Methods for sequencing nucleic acids are well known in the art and include the Sanger method of dideoxy-mediated chain termination (for example, see. Sanger et al., Proc. Natl. Acad. Sci. 74:5463, 1977; “DNA Sequencing” in Sambrook et al. (eds), Molecular Cloning: A Laboratory Manual (Second Edition), Plainview, N.Y.: Cold Spring Harbor Laboratory Press (1989)); the Maxam-Gilbert chemical degradation of DNA (Maxam and Gilbert, Methods Enzymol. 65:499 (1980); and “DNA Sequencing” in Sambrook et al., supra, 1989); and automated methods, for example, mass spectrometry methods.

“PAI-1 drug” means any drug that changes the levels or activity of a PAI-1. For example, the PAI-1 drug may be an agonist or antagonist of PAI-1. A drug which reduces or inhibits the levels or activity of PAI-1 is preferred. Examples of antagonists of PAI-1 include without limitation spironolactone, imidapril, an angiotensin converting enzyme inhibitor, captopril, enalapril, an angiotensin II receptor antagonist or blocker such as candesartan cilexetil, drugs that decrease endogenous NO, arginine and ramipril, benazepril, amlodipine/benazepril, statins, dietary isoprenoids, defibrotide, TNP-470 (fumagillin derivative, Abbott Laboratorie), Xigris (drotrecogin alfa; Eli Lilly & Co.), and RhEndostatin (EntreMed Inc.).

“TAFI drug” means any drug that changes the levels or activity of a TAFI. For example, the TAFI drug may be an agonist or antagonist of TAFI. A drug which reduces or inhibits the levels or activity of TAFI is preferred. Examples of antagonists of TAFI include without limitation potato carboxypeptidase inhibitor (PCI).

“tPA drug” means any drug that changes the levels or activity of tPA. For example, the tPA drug may be an agonist or antagonist of tPA. A drug which enhances or increases the levels or activity of tPA is preferred. Examples of tPA agonists include, without limitation, calcium channel blockers, amlodipine/benazepril, Solinase (pamiteplase; Astellas Pharma Inc., Toa Eiyo Ltd); Prolyse (Abbott Laboratories), Metalyse (tenecteplase) (Boehringer Ingelheim GmBH), Actilyse (Boehringer Ingelheim GmBH), Amediplase (Menarini Group), Activacin (alteplase) (Kyowa Hakko Kogyo Co. Ltd), Grtpa (Mitsubishi Pharma Corporation), Plasvata (Asahi Kasei Corporation), Indikinase (Bharat Biotech International Limited), Thromboflux (Bharat Serums and Vaccines Limited), Retavase (Biovail Corporation), Restor/Strikase (Claris Lifesciences Limited), Cleactor (monteplaser) (Eisai Co Ltd), Prokinase (Emcure Pharmaceuticals Ltd), Activase (alteplase) (Genentech Inc., Roche Holdings Ltd), TNKase (tenecteplase (Genentech Inc., Roche Holdings Ltd., Schering-Plough Corp), Mucolase (Hanmi Pharmaceutical Company Limited), Retavase (reteplase (Johnson & Johnson), Hapase Kowa (Kowa Co. Ltd), Icikinase (Nicholas Piramal India Limited), Streptase (Sanofi-Aventis), Sikaitong (Shanghai Fosun Pharmaceutical), Shankinase (Shantha Biotechnics), Eminase (Shire Plc, GlaxoSmithKline Plc), Oneplus (Zeria Pharmaceutical Co., Ltd.), and Retavase (PDL BioPharma Inc.).

“uPA drug” means any drug that changes the levels or activity of uPA. For example, the uPA drug may be an agonist or antagonist of uPA. In some aspects, a drug which reduces or inhibits the levels or activity of uPA is employed. In other aspects, a drug which enhances or increases the levels or activity of uPA is employed. Examples of uPA antagonists are described in US Published Application No. 20030232389, WO0216929, U.S. Pat. No. 6,200,989, and WO9906387.

### DETAILED DESCRIPTION

### Diagnostic Applications

The invention relates to the identification of individual's at risk of endometriosis by detecting allelic variation at one or more positions in one or more genes associated with the fibrinolytic pathway, optionally in combination with any other polymorphisms in the gene that is or becomes known.

Certain aspects of the invention stem from the observation that a polymorphism in the PAI-1 gene, a polymorphism in the tPA gene, a polymorphism in the uPA gene, and/or a polymorphism in the TAFI gene, are correlated with an individual's risk for endometriosis. Further aspects provide methods for detecting an individual's increased or decreased risk for endometriosis. Still further embodiments provide kits, reagents and arrays useful for detecting an individual's risk for endometriosis. The methods of the invention may be useful to assess the predisposition and/or susceptibility of an individual to endometriosis. A PAI-1 polymorphism, tPA polymorphism, uPA polymorphism, and/or TAFI polyporphism may be particularly relevant in the development of endometriosis and thus the present invention may be used to recognize individuals who are particularly at risk of developing these conditions.

In one aspect, the invention provides a method for detecting an individual's increased or decreased risk for endometriosis by detecting the presence of at least one endometriosis-associated polymorphism in one or more genes of the fibrinolytic pathway, in particular a PAI-1 gene, tPA gene, uPA gene, and/or TAFI gene, more particularly a PAI-1 locus and/or TAFI locus, in a nucleic acid sample of the individual, wherein the presence of the at least one polymorphism indicates the individual's increased or decreased risk for endometriosis.

A polymorphism can be any predisposing or protective polymorphism in a gene of the fibrinolytic pathway, in particular a PAI-1 gene, tPA gene, uPA gene, and/or TAFI gene, more particularly a PAI-1 locus and/or a TAFI locus. In an embodiment of the invention, a polymorphism can be any polymorphism identified as predisposing or protective by methods taught herein. In another embodiment, the polymorphism can be a single nucleotide polymorphism (SNP) in a gene of the fibrinolytic pathway, in particular a PAI-1 gene, tPA gene, uPA gene, and/or TAFI gene, more particularly a PAI-1 locus and/or TAFI locus. In a further embodiment, specific haplotypes in the PAI-1 locus and/or TAFI locus as well as specific combinations of, and interactions between, SNPs at this and other loci can be indicative of an increased or a decreased risk of endometriosis.

In an aspect, the presence of the polymorphism from only one parent is sufficiently predictive. In a further aspect, the presence of the polymorphism from both parents is sufficiently predictive.

In particular methods of the invention, the presence or absence of a variant nucleotide at position 837 of SEQ ID NO. 1 is detected, more particularly a single nucleotide polymorphism at position 837 which is the absence of a G is detected.

In other particular methods of the invention, the presence or absence of a variant nucleotide at position 1064 of SEQ ID NO. 9 is detected, in particular a C to T substitution.

In further particular methods of the invention, the presence or absence of a variant nucleotide at position −7351 of a tPA gene (e.g. SEQ ID NO. 23) is detected, in particular a T to C substitution.

In a still further particular method of the invention, a C→T polymorphism in exon 6 encoding the kringle domain of uPA is detected, in particular a C to T substitution at position 3007 in SEQ ID NO. 24.

In a still further particular method of the invention a T→C polymorphism in intron 7 of uPA is detected, in particular a T to C substitution at position 3637 in SEQ ID NO. 24.

The polymorphisms identified herein (for example, the polymorphism at position 837 of a PAI-1 gene and/or 1064/1040 of the TAFI gene) may be detected using analytical procedures well known to a person skilled in the art. Suitable methods for detection of allelic variation are described in standard textbooks (e.g. “Laboratory Protocols for Mutation Detection”, U. Landegren (ed), Oxford University and reviewed by Nollau et al, Clin. Chem. 43, 114-1120, 1997). Generally, a method for detecting a polymorphism comprises a mutation discrimination technique, optionally an amplification reaction, and a signal generation system.

Suitable mutation discrimination techniques include without limitation mutation detection techniques such as DNA sequencing, sequencing by hybridization, scanning (e.g. single-strand conformation polymorphism analysis (SSCP), denaturing gradient gel electrophoresis (DGGE), temperature gradient gel electrophoresis (TGGE), cleavase, heteroduplex analysis, chemical mismatch cleavage (CMC), enzymatic mismatch cleavage), solid phase hybridization [e.g. dot blots, multiple allele specific diagnostic assay (MASDA)], reverse dot blots, oligonucleotide arrays (DNA Chips)], solution phase hybridization [eg. Taqman (U.S. Pat. Nos. 5,210,015 and 5,487,972 to Hoffman-LA Roche), and Molecular Beacons (Tyagi et al, 1996, Nature Biotechnology, 14: 303, WO 95/13399)], extension based techniques [e.g. amplification refractory mutation system linear extension (ALEX™) (EP Patent No. EP 332435), amplification refractory mutation system (ARMST™) (EP Patent NO. 332435; U.S. Pat. No. 5,595,890; Newton et al Nucleic Acids Research 17:2503, 1989); competitive oligonucleotide priming system (COPS) (Gibbs et al, 1989, Nucleic Acid Research 17:2347); incorporation based techniques [e.g. mini-sequencing, arrayed primer extension (APEX)], restriction enzyme based techniques (e.g. restriction fragment length polymorphism, restriction site generating PCR), ligation based techniques (oligonucleotide ligation assay (OLA)—Nickerson et al, 1990, PNAS 87:8923-8927); real time PCR using a microfluidic lab-on-a chip system (Srinivasan V et al, Lab Chip. 2004 4(4):310), and other techniques known in the art.

Suitable signal generation or detection systems that may be used in combination with the mutation discrimination techniques include without limitation fluorescence, fluorescence resonance energy transfer, fluorescence quenching, fluorescence polarization (UK Patent No. 2228998), chemiluminescence, electrochemiluminescence, raman, radioactivity, colorimetric, hybridization protection assay, mass spectrometry, and surface enhanced raman resonance spectroscopy (WO 97/05280).

Various amplification methods known in the art can be used to detect nucleotide changes in a target nucleic acid. Suitable amplification methods include polymerase chain reaction (PCR), self sustained replication, branched DNA (b-DNA), ligase chain reaction (LCR), nucleic acid sequence based amplification (NASBA), and strand displacement amplification (SDA). Polymerase chain reaction (PCR), is well known in the art [See U.S. Pat. Nos. 4,683,195; 4,683,202; 4,965,188; PCR Applications, 1999, (Innis et al., eds., Academic Press, San Diego), PCR Strategies, 1995, (Innis et al., eds., Academic Press, San Diego); PCR Protocols, 1990, (Innis et al., eds., Academic Press, San Diego); and PCR Technology, 1989, (Erlich, ed., Stockton Press, New York); Abramson et al., 1993, Current Opinion in Biotechnology, 4:41-47; commercial vendors include PE Biosystems (Foster City, Calif.)]. Reverse-transcription-polymerase chain reaction (RT-PCR) is also well known in the art and for example, is described in U.S. Pat. Nos. 5,310,652; 5,322,770; 5,561,058; 5,641,864; and 5,693,517. Other known amplification methods include the ligase chain reaction (Wu and Wallace, 1988, Genomics 4:560-569); the strand displacement assay (Walker et al., 1992, Proc, Natl. Acad. Sci. USA 89:392-396, Walker et al. 1992, Nucleic Acids Res. 20:1691-1696, and U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., 1989, Proc. Natl. Acad. Sci. USA, 86:1173-1177); and self-sustained sequence replication (3SR) (Guatelli et al., 1990, Proc. Natl. Acad. Sci. USA, 87:1874-1878 and WO 92/08800). Methods that amplify a probe to detectable levels can also be used, including QB-replicase amplification (Kramer et al., 1989, Nature, 339:401-402, and Lomeli et al., 1989, Clin. Cheni. 35:1826-1831).

Certain methods of the invention may employ restriction fragment length analysis, sequencing, hybridization, an oligonucleotide ligation assay, polymerase proofreading methods, allele-specific PCR and reading sequence data. Particular methods of the invention are described below.

A diagnostic method of the invention may comprise (a) contacting a nucleic acid sample with one or more oligonucleotides that hybridize under stringent hybridization conditions to at least one polymorphism of a gene of the fibrinolytic pathway (e.g. PAI-1 polymorphism, in particular, absence of a G at position 837 of SEQ ID NO. 1 and/or a Thr325Ile mutation in the TAFI gene) and detecting the hybridization; (b) detection of at least one polymorphism by amplification of the nucleic acid sample by, for example, PCR; or (c) detection of at least one polymorphism by direct sequencing of the nucleic acid sample.

In certain aspects, an individual's risk for endometriosis is diagnosed from the individual's genotype, in particular a PAI-1, tPA, uPA, and/or TAFI genotype, more particularly the individual's PAI-1 genotype. An individual who has at least one polymorphism statistically associated with endometriosis possesses a factor contributing to either an increased or a decreased risk as compared to an individual without the polymorphism. A statistical association of various polymorphisms (sequence variants) with endometriosis is shown in the Examples. A genotype can be determined using any method capable of identifying nucleotide variation, e.g., nucleotide variation consisting of single nucleotide polymorphic sites. A number of suitable methods are described herein. For example, genotyping may be carried out using oligonucleotide probes specific to variant PAI-1 sequences, tPA sequences, uPA sequences, and/or TAFI sequences. In an aspect, a region of the PAI-1 gene, a tPA gene, uPA gene and/or TAFI gene which encompasses a polymorphic site of interest is amplified prior to, or concurrent with, the hybridization of probes complementary to such sites. In the alternative, allele-specific amplification or extension reactions with allele-specific primers are used which support primer extension if the targeted allele is present. Typically, an allele-specific primer hybridizes to a gene of the fibrolytic pathway, including a PAI-1 gene, tPA gene, uPA gene, or TAFI gene, such that the 3′ terminal nucleotide aligns with a polymorphic position. In particular aspects of the invention, the primers for the PAI-1 gene are the oligonucleotides of SEQ ID NOs. 5, 6, 7, and 8; the primers for the TAFI gene are the oligonucleotides of SEQ ID NOs. 10 and 11; the primers for the tPA gene are the oligonucleotides of SEQ ID NOs. 12, 13, and 14; and, the primers for the uPA gene are the oligonucleotides of SEQ ID NOs.15, 16, 17 and 18 (C to T) and 19, 20, 21, and 22 (T to C).

One aspect of the invention provides nucleic acids, for example, nucleic acids comprising one or more novel polymorphisms in a gene associated with endometriosis and/or oligonucleotides useful for detecting such polymorphisms. Accordingly, one embodiment of the invention is an isolated nucleic acid molecule comprising a portion of such gene, its complement, and/or a variant thereof. In particular aspects, the variant comprises a polymorphisin identified herein. More particularly, the variant comprises at least one of the polymorphisms identified herein to be associated with endometriosis. In a further embodiment, the nucleic acid molecule comprises or consists of a primer and/or a probe specific to at least one of the polymorphisms identified in a gene of the fibrinolytic pathway, in particular a PAI-1 gene, tPA gene, uPA gene, and/or TAFI gene, more particularly a PAI-1 gene (e.g., those identified herein to be associated with endometriosis).

In one aspect, the invention provides a method for detecting an individual's increased or decreased risk for endometriosis by detecting the presence of one or more SNPs in a nucleic acid sample of the individual, wherein the presence of the SNP(s) indicates the individual's increased or decreased risk for endometriosis. The SNPs can be any SNPs in a gene of the fibrinolytic pathway, in particular a PAI-1 gene, tPA gene, uPA gene, and/or TAFI gene, more particularly a PAI-1 locus, including SNPs in exons, introns and/or upstream and/or downstream regions. In particular aspects, the SNPs are in a regulatory region, more particularly a promoter or enhancer region. Examples of SNPs include, but are not limited to, those discussed in detail herein and in the Examples. In one embodiment, the SNPs present in a PAI-1 locus (or locus of another gene of the fibrinolytic pathway) are identified by genotyping the PAI-1 SNPs. In another embodiment, the SNPs present in a TAFI locus, tPA locus, and/or uPA locus (or locus of another gene of the fibrinolytic pathway) are identified by genotyping the TAFI, tPA, and/or uPA SNPs. In certain embodiments, the genotype of one SNP can be used to determine an individual's risk for endometriosis. In other embodiments, the genotypes of a plurality of SNPs can be used. In other embodiments, certain combinations of SNPs at either the same or different loci can be used.

Genotyping can also be carried out by detecting and analyzing mRNA under conditions when both maternal and paternal chromosomes are transcribed. Amplification of RNA can be carried out by first reverse-transcribing the target RNA using, for example, a viral reverse transcriptase, and then amplifying the resulting cDNA, or using a combined high-temperature reverse-transcription-polymerase chain reaction (RT-PCR) [see for example, U.S. Pat. Nos. 5,310,652; 5,322,770; 5,561,058; 5,641,864; and 5,693,517 and Myers and Sigua, 1995, in PCR Strategies, supra, Chapter 5).

Alleles can also be identified using allele-specific amplification or primer extension methods which are based on the inhibitory effect of a terminal primer mismatch on the ability of a DNA polymerase to extend the primer (see, for example, U.S. Pat. Nos. 5,137,806; 5,595,890; 5,639,611; and U.S. Pat. No. 4,851,331). To detect an allele sequence using these methods, a primer complementary to the gene is selected such that the 3′ terminal nucleotide hybridizes at the polymorphic position (e.g., position 837 of PAI-1—SEQ ID NO. 1). Examples of primers for a PAI-1 polymorphism are the oligonucleotides in SEQ ID NOs. 5, 6, 7, and 8; examples of primers for a TAFI polymorphism are SEQ ID NOs.10 and 11; examples of primers for a tPA polymorphism are SEQ ID NOs.12, 13 and 14; and examples of primers for a uPA polymorphism are SEQ ID NOs.15, 16, 17, and 18 (C to T) and 19, 20, 21, and 22 (T to C). In the presence of the allele to be identified, the primer matches the target sequence at the 3′ terminus and the primer is extended. In the absence of the allele, the primer has a 3′ mismatch relative to the target sequence and primer extension is either eliminated or significantly reduced. Using allele-specific amplification-based genotyping, identification of the alleles requires the detection of the presence or absence of amplified target sequences. Methods for the detection of amplified target sequences are well known in the art and include, for example, gel electrophoresis (see Sambrook et al., 1989, infra) and the probe hybridization assays described herein.

Allele-specific amplification-based methods of genotyping can facilitate the identification of haplotypes. Essentially, the allele-specific amplification is used to amplify a region encompassing multiple polymorphic sites from only one of the two alleles in a heterozygous sample. The SNP variants present within the amplified sequence are then identified by probe hybridization or sequencing.

A kinetic-PCR method in which the generation of amplified nucleic acid is detected by monitoring the increase in the total amount of double-stranded DNA in the reaction mixture can also be used to identify polymorphisms. The method is described, for example, in Higuchi et al., 1992, BioTechnology, 10:413-417; Higuchi et al., 1993, BioTechnology, 11: 10261030; Higuchi and Watson, in PCR Applications, supra, Chapter 16; U.S. Pat. Nos. 5,994,056 and 6,171,785; and European Patent Publication Nos. 487,218 and 512,334. The detection of double-stranded target DNA depends on the increased fluorescence that DNA-binding dyes, such as ethidium bromide or SYBR Green, exhibit when bound to double-stranded DNA. An increase of double-stranded DNA produced from the synthesis of target sequences provides an increase in the amount of dye bound to double-stranded DNA and a concomitant detectable increase in fluorescence. In the kinetic-PCR methods, amplification reactions are carried out using a pair of primers specific for one of the alleles, such that each amplification indicates the presence of a particular allele. For example, by performing two amplifications, one using primers specific for the wild-type allele (e.g., PAI-1 oligonucleotides of SEQ ID NOs. 6 and 7) and one using primers specific for the mutant allele (e.g., PAI-1 oligonucleotides of SEQ ID NOs. 6 and 8), the genotype of the sample with respect to that SNP can be determined.

Alleles may also be identified using probe-based methods which rely on the difference in stability of hybridization duplexes formed between a probe and its corresponding target sequence comprising an allele. Under sufficient stringent hybridization conditions, stable duplexes are formed only between a probe and its target allele sequence and not other allele sequences. The presence of stable hybridization duplexes can be detected by methods known in the art.

Probes suitable for use in the probe-based methods of the invention, which contain a hybridizing region either substantially complementary or exactly complementary to a polymorphic site of a gene of the fibrinolytic pathway, in particular a PAI-1 gene, tPA gene, uPA gene, and/or TAFI gene, more particularly a PAI-1 gene and/or a TAFI gene, or the complement thereof can be selected using the guidance provided herein and well known in the art. Similarly, suitable hybridization conditions (e.g., stringent conditions), which depend on the exact size and sequence of the probe, can be selected empirically using the guidance provided herein and well known in the art (see, e.g., Nucleic Acid Hybridization (B. D. Haines and S. F. Higgins. eds., 1984) and Sambrook et al., Molecular Cloning—A Laboratory Manual (3rd Ed.), Vol. 1-3, Cold Spring Harbor Laboratory, Cold Spring Harbor, N.Y., 2000).

In aspects of probe-based methods for determining genotypes, multiple nucleic acid sequences from the genes which comprise the polymorphic sites are amplified and hybridized to a set of probes under stringent conditions. The alleles present are determined from the pattern of binding of the probes to the amplified target sequences. In this aspect, amplification is carried out to provide sufficient nucleic acid for analysis by probe hybridization. Therefore, primers are designed to amplify the regions of the genes encompassing the polymorphic sites regardless of the allele present in the sample. Primers which hybridize to conserved regions of the genes are used for allele-independent amplification. Suitable allele-independent primers can be selected routinely (see, for example, the primers of SEQ ID NOs. 5, 6, 7, and 8 for a PAI-1 polymorphism). Assays suitable for detecting hybrids formed between probes and target nucleic acid sequences in a sample are well known in the art, for example, immobilized target (dot-blot) and immobilized probe (reverse dot-blot or line-blot) assay formats. (See, for example, Schollen et al, Clin. Chem. 43: 18-23, 2997; Gilles et al, Nat. Biotechnol. 1999, 17(40:365-70; and U.S. Pat. Nos. 5,310,893, 5,451,512, 5,468,613 and 5,604,099 for dot blot and reverse dot blot assay formats).

In an aspect of the invention, probe-based genotyping can be carried out using a 5′-nuclease assay. (See for example, Holland et al., 1988, Proc. Natl. Acad. Sci. USA, 88:7276-7280, and U.S. Pat. Nos. 5,108,892, 5,210,015, 5,487,972, and 5,804,375 describing 5′-nuclease assays). In this assay, labeled detection probes that hybridize within the amplified region are added during the amplification reaction mixture. The probes are modified so that they do not act as primers for DNA synthesis. The amplification is carried out using a DNA polymerase that possesses 5′ to 3′ exonuclease activity. Any probe which hybridizes to the target nucleic acid downstream from the primer being extended in the amplification is degraded by the 5′ to 3′ exonuclease activity of the DNA polymerase. Therefore, the synthesis of a new target strand also results in the degradation of a probe, and the accumulation of the probe degradation product provides a measure of the synthesis of target sequences. Any method suitable for detecting the products may be used in the assay. In an aspect of the invention, the probes are labeled with two fluorescent dyes, one of which is capable of quenching the fluorescence of the other dye. The dyes are attached to the probe (e.g., one is attached to the 5′ terminus and the other is attached to an internal site), such that quenching occurs when the probe is not hybridized and the cleavage of the probe by the 5′ to 3′ exonuclease activity occurs in between the two dyes. Amplification results in cleavage of the probe between the dyes with the elimination of quenching and an increase in the fluorescence which is observable from the initially quenched dye. The accumulation of degradation product is determined by measuring the increase in reaction fluorescence. A 5-nuclease assay may employ allele-specific amplification primers such that the probe is used only to detect the presence of amplified product. Alternatively, the 5′-nuclease assay can employ a target-specific probe.

The methods described above typically employ labeled oligonucleotides to facilitate detection of the hybrid duplexes. Oligonucleotides can be labeled by incorporating a label detectable by spectroscopic, photochemical, biochemical, immunochemical, radiological, radiochemical or chemical means. Useful labels and methods for labeling oligonucleotides are described herein.

The methods of the invention may also comprise detecting other markers and polymorphisms associated with endometriosis. Other markers include the following genes and gene products aberrantly expressed in the endometrium from subjects with endometriosis: aromatase, endometrial bleeding factor, hepatocyte growth factor, 17-β-hydroxysteroid dehydrogenase, HOX A10, HOX All, leukaemia inhibitory factor, interleukin 6, C3 complement, matrix metalloproteinases 3, 7, and 11, tissue inhibitors of metalloproteinases, progesterone-receptor and isoforms, complement 3, catalase, thrombospondin 1, vascular endothelial growth factor, integrin αᵥβ₃, glycodelin, cytochrome P450 1A1, N-acetyl-transferase 2, glutathione-S-transferase M1, T1, glutathione peroxidase, EBAF, VEGF, mucins, galactose-1-phosphate uridyl transferase, oestrogen receptor, androgen receptor, PTEN, P53, and peroxisome proliferators-activated receptor γ2 Pro-12-Ala allele.

Evaluation of a candidate gene for association with various phenotypes pertaining to endometriosis is described in the Examples. In addition, design and execution of various types of association studies have been described in the art; (see, e.g., Handbook of Statistical Genetics, John Wiley and Sons Ltd.; Borecki and Suarez, 2001, Adv Genet 42:45-66; Cardon and Bell, 200 1, Nat Rev Genet 2:91-99; and Risch, 2000, Nature 405:847). Association studies have been used to evaluate candidate genes for association with a phenotypic trait (e.g., Thomsberry et al., 2001, Nature Genetics 28:286-289) and to perform whole genome scans to identify genes that contribute to phenotypic variation.

### Kits

The invention relates to a kit useful for detecting the presence of a predisposing or a protective polymorphism in a gene of the fibrinolytic pathway, in particular a PAI-1 gene, tPA gene, uPA gene, and/or TAFI gene, more particularly a PAI-1 locus and/or a TAFI locus, in a nucleic acid sample of an individual whose risk or susceptibility for endometriosis is being assessed. The kit can comprise one or more oligonucleotides capable of detecting a predisposing or protective polymorphism in the locus as well as instructions for using the kit to detect risk or susceptibility to endometriosis. In embodiments, the oligonucleotide or oligonucleotides each individually comprise a sequence that hybridizes under stringent conditions to at least one polymorphism [e.g. a PAI-1 polymorphism at position 837 and/or a TAFI polymorphism at position 1064/1060 (i.e., a Thr325Ile mutation)]. In some embodiments, the oligonucleotide or oligonucleotides each individually comprise a sequence that is fully complementary to a nucleic acid sequence comprising a polymorphism described herein.

In aspects of the invention, the oligonucleotide can be used to detect the presence of a polymorphism by hybridizing to the polymorphism under stringent conditions. In some embodiments, the oligonucleotide can be used as an extension primer in either an amplification reaction such as PCR or a sequencing reaction, wherein a polymorphism is detected either by amplification or sequencing. In particular embodiments, the kit also comprises amplification or sequencing primers which can, but need not, be sequence-specific. The kit can also comprise reagents for labeling one or more of the oligonucleotides, or comprise labeled oligonucleotides. A kit can optionally comprise reagents to detect the label.

In some embodiments, the kit can comprise one or more oligonucleotides that can be used to detect the presence of two or more predisposing or protective endometriosis associated polymorphisms or combinations of predisposing and protective polymorphisms or both.

The invention also relates to a kit comprising a container unit and components for practicing a method of the invention. A kit can contain oligonucleotide probes specific for alleles as well as instructions for their use to determine risk or susceptibility for endometriosis. In some cases, a kit may comprise detection probes fixed to an appropriate support membrane. The kit can also contain amplification primers for amplifying regions of a locus encompassing the polymorphic sites, as such primers are useful in aspects of the invention. Alternatively, useful kits can contain a set of primers comprising an allele-specific primer for the specific amplification of alleles. Other optional components of the kits include additional reagents used in the genotyping methods as described herein. For example, a kit additionally can contain an agent to catalyze the synthesis of primer extension products, substrate nucleoside triphosphates, reagents for labeling and/or detecting nucleic acids (for example, an avidin-enzyme conjugate and enzyme substrate and a chromogen if the label is biotin) and appropriate buffers for amplification or hybridization reactions.

One aspect of the invention provides kits for detecting the presence of a first predisposing or protective endometriosis associated polymorphism in a gene, e.g., in a nucleic acid sample of an individual whose susceptibility or risk for endometriosis is being assessed. Thus, an aspect of the invention provides a kit including one or more oligonucleotides capable of detecting a polymorphism and instructions for detecting the polymorphism with the oligonucleotides and for correlating the detection to the individual's risk for endometriosis, packaged in one or more containers.

Generally all of the features disclosed herein for the methods of the invention apply to the kits of the invention. For example, in a particular class of embodiments, the polymorphism is defined by position 837 and optionally flanking sequences, of a PAI-1 gene comprising SEQ ID NO. 1. In another particular class of embodiments, the polymorphism is defined by position 1064, and optionally flanking sequences, of a TAFI gene comprising SEQ ID NO. 9.

In one aspect, the kit can be used to detect the presence of a polymorphism by hybridization of a nucleic acid probe to the polymorphism. Therefore, in an aspect of the invention, the oligonucleotides comprise at least one probe. In certain embodiments, the oligonucleotide hybridizes under stringent conditions to a region of a gene comprising an endometriosis-associated polymorphism. In another aspect, the polymorphism is a single nucleotide polymorphism comprising a nucleotide at a particular nucleotide position. Under stringent conditions, the oligonucleotide hybridizes to a region of a gene comprising the single nucleotide polymorphism with a signal to noise ratio that is at least 2 times (e.g., at least 5 times or at least 10 times) the signal to noise ratio at which the oligonucleotide hybridizes to the region of the gene in the absence of the polymorphism at the nucleotide position. The oligonucleotide is typically fully complementary to the region of the gene comprising the polymorphism, and typically comprises at least about 10 to 30 contiguous nucleotides complementary to the gene.

To facilitate detection of a polymorphism the oligonucleotides in a kit optionally comprise a label (e.g., an isotopic, fluorescent, fluorogenic, luminescent or colorimetric label). In some aspects, the label itself directly produces a detectable signal (e.g., a fluorescent label). In other aspects, the kit also includes a reagent that detects the label (e.g., an enzyme that cleaves a colorimetric label and the like).

In an aspect of a kit of the invention, the oligonucleotides comprise primers. The primer(s) can be used to detect a polymorphism, e.g., in an allele-specific amplification or extension reaction. The primer(s) can be used to amplify a region of a gene comprising the polymorphism for subsequent detection of the polymorphism by hybridization, sequencing, or the like. Thus, in one aspect, the oligonucleotides comprise amplification primers, wherein the amplification primers amplify a nucleic acid sequence comprising the polymorphism. The oligonucleotides can also comprise primers that flank the polymorphism. In particular aspects a kit comprises primers for a PAI-1 polymorphism (e.g., primers comprising SEQ ID NOs. 5, 6, 7, and 8); primers for a TAFI polymorphism (e.g., primers comprising SEQ ID NOs.10 and 11); primers for a tPA polymorphism (e.g., primers comprising SEQ ID NOs.12, 13 and 14); and/or primers for a uPA polymorphism [e.g., primers comprising SEQ ID NOs.15, 16, 17, and 18 (C to T) and 19, 20, 21, and 22 (T to C)].

A kit can optionally be used to detect more than one polymorphism (simultaneously or sequentially). Thus, in aspects of the invention, the kit also includes one or more second oligonucleotides capable of detecting a second polymorphism (and optionally third, fourth, fifth, etc. oligonucleotides capable of detecting third, fourth, fifth, etc. polymorphisms). A second polymorphism can be at the same polymorphic site as a first polymorphism, or at a different polymorphic site (e.g. in a PAI-1, TAFI, tPA, uPA or a different gene), and can be protective or predisposing.

The oligonucleotides in a kit can be optionally immobilized on a substrate. The substrate can be, for example, a planar substrate or a beaded substrate.

### Array

The invention relates to an array, a support with immobilized oligonucleotides, useful for practicing the present method. A useful array can contain oligonucleotide probes specific for alleles or certain combinations of alleles described herein. The oligonucleotides can be immobilized on a substrate, e.g., a membrane or glass. The oligonucleotides may be labeled. In some embodiments, the array can be a micro-array. In some embodiments, the array can comprise one or more oligonucleotides used to detect the presence of two or more alleles or certain combinations of alleles.

Oligonucleotide(s) can be arranged in an array of other oligonucleotides used to detect other polymorphisms, e.g., other polymorphisms in PAI-1, TAFI, tPA, uPA or other genes in the fibrinolytic pathway.

In aspects of the invention, arrays are provided for detecting the presence of one or more predisposing and/or protective endometriosis-associated polymorphisms in one or more genes, for example, in a nucleic acid sample of an individual whose risk for endometriosis is being assessed. In a particular aspect, the array comprises a substrate and a plurality of oligonucleotides, each oligonucleotide capable of hybridizing to a region of a gene comprising at least one of polymorphism (e.g. a single nucleotide polymorphism at position 837 of PAI-1; a Thr325Ile mutation of a TAFI gene; a −7351C/T polymorphism of a tPA gene; and/or, C→T and/or T→C polymorphisms in exon 6 encoding the kringle domain and in intron 7, respectively of the uPA gene). The hybridization detects the presence of the polymorphism, and provides an indication of the individual's risk for endometriosis. Typically, the array is used for detecting the presence of a plurality of polymorphisms, e.g., multiple alleles at a single polymorphic site and/or different polymorphic sites.

Generally all of the features noted for the method and kit aspects of the invention apply to an array of the invention. For example, the one or more polymorphisms preferably comprise one or more single nucleotide polymorphisms. For example, the polymorphism may be a single nucleotide polymorphism at position 837 of SEQ ID NO. 1 and/or a polymorphism may be a single nucleotide polymorphism at position 1064 of SEQ ID NO. 9.

The invention in particular contemplates an array that can be used to detect the presence of one or more SNPs comprising oligonucleotides which are capable of hybridizing under stringent conditions to a region of a gene comprising a single nucleotide polymorphism with a signal to noise ratio that is at least 2, 5 or 10 times that at which the oligonucleotide hybridizes to a region of the gene comprising another single nucleotide polymorphism. Typically, one oligonucleotide is used to detect one SNP; that is, each oligonucleotide is capable of hybridizing to a distinct single nucleotide polymorphism.

A plurality of oligonucleotides may be immobilized on a substrate, e.g., a planar substrate, a membrane, a glass slide, or the like. Typically, each of the plurality of oligonucleotides is immobilized at a known, pre-determined position on the substrate. Each oligonucleotide may be bound (e.g., electrostatically or covalently bound, directly or via a linker) to the substrate at a unique location. In order to facilitate detection of polymorphisms by specific hybridization with the oligonucleotides, each of the oligonucleotides is typically fully complementary to a region of a gene comprising one of the polymorphisms, and each of the plurality of oligonucleotides comprises at least about 10 to 30 contiguous nucleotides complementary to the gene. An oligonucleotide may optionally comprise a label that facilitates detection of hybridization between the oligonucleotide and the polymorphism.

An array can be part of a system. Thus, the invention provides a system comprising an array of the invention and system instructions that correlate the detection of the presence of one or more predisposing or protective endometriosis-associated polymorphisms to the individual's risk for endometriosis.

Methods of making, using, and analyzing arrays such as micro-arrays are well known in the art (see, e.g., Wang et al., 1998, Science 280:1077-82; Lockhart and Winzeler, 2000, Nature 405:827-836; and Scherf et al., 2000, Nat Genet. 24:236). Arrays can be formed (e.g., printed), using commercially available instruments (e.g., a GMS 417 Arrayer, Affymetrix, Santa Clara, Calif.). Suitable solid supports are commercially available and include without limitation membranes (e.g., nylon, PVDF, and nitrocellulose membranes) and surface-modified and pre-coated slides with a variety of surface chemistries (e.g., from TeleChem International (www.arrayit.com), Corning, Inc. (Corning, N.Y.), or Greiner Bio-One, Inc. (www.greinerbiooneinc.com). Further, custom arrays of nucleic acids are commercially available (e.g., from Agilent Technologies (CA, USA) and from TeleChem International (CA, USA) (www.arrayit.com)).

### Automated Methods

Various automated systems can be used to perform some or all of the methods of the invention. In addition, digital or analog systems, for example, comprising a digital or analog computer, can also control a variety of other functions such as a user viewable display to permit viewing of method results by a user and/or control of output features. For example, particular methods described herein may be implemented on a computer program or programs. The programs may correlate detection of the presence of one or more predisposing or protective polymorphisms to an individual's risk for endometriosis. Therefore, the invention contemplates digital systems, including computers, computer readable media, and/or integrated systems comprising instructions (e.g., embodied in appropriate software) for performing the methods of the invention. In an aspect, the invention provides a digital system comprising instructions for correlating detection of the presence of one or more predisposing or protective polymorphisms to an individual's risk for endometriosis. A digital system may also include information corresponding to individual genotypes for a set of genetic markers, phenotypic information, and the like. The system may also assist in the detection of polymorphisms by, for example, controlling a microarray scanner.

Standard desktop applications can be adapted to the present invention by inputting data which is loaded into the memory of a digital system, and performing an operation on the data. Such applications include word processing software such as Microsoft Word, database software such as Microsoft Excel, and/or database programs such as Microsoft Access. For example, systems including these software applications containing appropriate genotypic information, associations between phenotype and genotype, and other relevant information may be used in conjunction with a user interface (e.g., a GUI in a standard operating system such as a Windows, Macintosh or LINUX system) to perform any analysis noted herein, or simply to retrieve data (e.g., in a spreadsheet) to be used in the methods disclosed herein. A system may include a digital computer with software for performing association analysis and/or risk prediction, and also data sets entered into the software system comprising genotypes for a set of genetic markers, phenotypic values and the like. The systems of the invention can use commercially available computers.

The methods of the invention may also be embodied within the circuitry of an application specific integrated circuit (ASIC) or programmable logic device (PLD). In such applications, a method of the invention is embodied in a computer readable descriptor language that can be used to create an ASIC or PLD. The methods of the invention can also be embodied within the circuitry or logic processors of other digital apparatus, such as PDAs, laptop computer systems, displays, image editing equipment, and the like.

### Applications

A polymorphism that occurs in the promoter or enhancer region (e.g. position 837 of the PAI-1 gene and/or −7351 of tPA) is not expected to alter the amino acid sequence of the encoded polypeptide (e.g. PAI-1 and/or tPA), but may affect transcription and/or message stability of the sequences. Assays (e.g. reporter-based assays) may be devised to detect whether one or more polymorphism affects transcription and/or message stability.

Individuals who carry allelic variants in a region of a gene associated with endometriosis (e.g. PAI-1 gene and/or TAFI gene) may exhibit differences in polypeptide levels under different physiological conditions and may display altered abilities to react to endometriosis. Further, differences in polypeptide levels resulting from allelic variation may have an effect on the response of an individual to drug therapy. For example, PAI-1 and/or TAFI polymorphisms may have an effect on the efficacy of drugs designed to modulate the activity of PAI-1 and/or TAFI. The polymorphisms may also affect the response to agents acting on other pathways regulated by PAI-1 and/or TAFI. Thus, the diagnostic methods of the invention may be useful to assess the efficacy of therapeutic compounds in the treatment of endometriosis, predict the clinical response to a therapeutic compound, and/or to determine therapeutic dose.

In an aspect, the invention provides a method for determining the efficacy of a treatment for a particular patient with endometriosis based on genotype comprising (a) determining the genotype for one or more polymorphism sites in a gene of the fibrinolytic pathway associated with endometriosis (e.g. the PAI-1 gene, tPA gene, uPA gene and/or TAFI gene) for a group of patients receiving a treatment; (b) sorting patients into subgroups based on their genotype; (c) identifying correlations between the subgroups and the efficacy of the treatment in the patients, (d) determining the genotype for the same polymorphism sites in the gene of the particular patient and determining the efficacy of the treatment for the particular patient based on a comparison of the genotype with the correlations identified in (c).

A PAI-1 drug, tPA drug, uPA drug and/or TAFI drug may be used to treat endometriosis in a subject. In some aspects, antagonists to polypeptides encoded by genes of the fibrinolytic pathway associated with endometriosis, in particular PAI-1, uPA and/or TAFI, can be used in the treatment of endometriosis. Antagonists can be antibodies, peptides, proteins, nucleic acids, small organic molecules, or polymers. In an aspect of the invention, the antagonist is an antibody. In other aspects, agonists to polypeptides encoded by genes of the fibrinolytic pathway associated with endometriosis, in particular tPA, can be used in the treatment of endometriosis. Agonists can be peptides, proteins, nucleic acids, small organic molecules, or polymers.

Antagonists or agonists may be prepared as compositions with a pharmaceutically acceptable carriers or diluents. Carriers and diluents include any and all solvents, dispersion media, microcapsules, liposomes, cationic lipid carriers, antibacterial agents, antifungal agents, isotonic and absorption delaying agents, and the like, which are compatible with the antagonists or agonists.

Antibodies for use in the present invention include but are not limited to monoclonal or polyclonal antibodies, immunologically active fragments (e.g. a Fab or (Fab)₂ fragments), antibody heavy chains, humanized antibodies, antibody light chains, genetically engineered single chain Fᵥ molecules, chimeric antibodies, for example, antibodies which contain the binding specificity of murine antibodies, but in which the remaining portions are of human origin, or derivatives, such as enzyme conjugates or labeled derivatives. The antibody can be chemically linked to another organic or bio-molecule.

Antibodies including monoclonal and polyclonal antibodies, fragments and chimeras, may be prepared using methods known to those skilled in the art. See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. Antibodies may also be obtained from scientific or commercial sources.

Peptide, protein, nucleic acid, small organic molecule, and polymer antagonists and agonists may be identified using methods known in the art including combinatorial methods.

Known antagonists may be used in the treatment of endometriosis. For example known antagonists of PAI-1, including without limitation, spironolactone, imidapril, angiotensin converting enzyme inhibitors (ACEI, captopril, or enalapril), angiotensin II receptor antagonist (AIIRA), or defibrotide (a polydeoxyribonucleotide) may be used to treate endometriosis. Antisense nucleic acid molecules may also be used to reduce the levels of PAI-1, uPA, and/or TAFI in treating endometriosis.

Known tPA agonists may be used in the treatment of endometriosis. For example known agonists of tPA include without limitation calcium channel blockers, amlodipine/benazepril, Solinase (pamiteplase; Astellas Pharma Inc., Toa Eiyo Ltd); Prolyse (Abbott Laboratories), Metalyse (tenecteplase) (Boehringer Ingelheim GmBH), Actilyse (Boehringer Ingelheim GmBH), Amediplase (Menarini Group), Activacin (alteplase) (Kyowa Hakko Kogyo Co. Ltd), Grtpa (Mitsubishi Pharma Corporation), Plasvata (Asahi Kasei Corporation), Indikinase (Bharat Biotech International Limited), Thromboflux (Bharat Serums and Vaccines Limited), Retavase (Biovail Corporation), Restor/Strikase (Claris Lifesciences Limited), Cleactor (monteplaser) (Eisai Co Ltd), Prokinase (Emcure Pharmaceuticals Ltd), Activase (alteplase) (Genentech Inc., Roche Holdings Ltd), TNKase (tenecteplase (Genentech Inc., Roche Holdings Ltd., Schering-Plough Corp), Mucolase (Hanmi Pharmaceutical Company Limited), Retavase (reteplase (Johnson & Johnson), Hapase Kowa (Kowa Co. Ltd), Icikinase (Nicholas Piramal India Limited), Streptase (Sanofi-Aventis), Sikaitong (Shanghai Fosun Pharmaceutical), Shankinase (Shantha Biotechnics), Eminase (Shire Plc, GlaxoSmithKline Plc), Oneplus (Zeria Pharmaceutical Co., Ltd.), and Retavase (PDL BioPharma Inc.)

An aspect of the invention is directed towards a method to treat endometriosis. The method may comprise selecting a subject diagnosed with endometriosis and administering to the subject a PAI-1 drug, tPA drug, uPA drug and/or TAFI drug. In a particular aspect, a plasminogen activator inhibitor-1 antagonist, a tPA agonist, an uPA antagonist or agonist, and/or a TAFI antagonist are administered. A drug or agonist or antagonist can be administered at a concentration suitable to reduce the effects of endometriosis. The concentration of the drug or agonist or antagonist is preferably less than about 100 μM, about 10 μM, about 1 μM, about 0.1 μM, about 0.01 μM, about 0.001 μM or about 0.0001 μM. The administering step can be performed by any acceptable means, including oral, inhalation, topical, intravenous, intraperitoneal, and intramuscular administration

An additional aspect of the invention is directed to methods for the prevention of endometriosis. The methods may comprise selecting a subject, and administering to the mammal a PAI-1 drug, tPA drug, uPA drug and/or TAFI drug. In a particular aspect, a plasminogen activator inhibitor-1 antagonist, tPA agonist, uPA antagonist or agonist, and/or a TAFI antagonist is administered. A drug or agonist or antagonist is preferably administered at a concentration suitable to reduce the occurrence or effects of endometriosis relative to a subject which did not receive the administration. The concentration of the drug or agonist or antagonist is preferably less than about 100 μM, about 10 μM, about 1 μM, about 0.1 μM, about 0.01 μM, about 0.001 μM or about 0.0001 μM. The administering step can be performed by any acceptable means, including oral, inhalation, topical, intravenous, intraperitoneal, and intramuscular administration. The plasminogen activator inhibitor-1 antagonist, tPA agonist, uPA antagonist or agonist, and/or TAFI antagonist can generally be any plasminogen activator inhibitor-1 antagonist, tPA agonist, uPA antagonist or agonist, and/or TAFI antagonist, respectively. In an aspect, the plasminogen activator inhibitor-1 antagonist, tPA agonist, uPA antagonist or agonist, and/or TAFI antagonist is an antibody, a protein, a peptide, a polynucleotide, or a small organic molecule. Examples of agonists and antagonists are described herein.

Another aspect embodiment of the invention is directed to the use of compounds that change the concentration of upstream regulators or downstream effector molecules of PAI-1, tPA, uPA and/or TAFI, in treating or preventing endometriosis. The method can comprise selecting a subject diagnosed with endometriosis, and administering to the mammal one or more agents. The agents can comprise urokinase, tissue plasminogen activator, vitronectin, plasminogen, plasmin, matrix metalloproteinases, or tissue inhibitors of metalloproteinases. The concentration of compound is preferably less than about 100 μM, about 10 μM, about 1 μM, about 0.1 μM, about 0.01 μM, about 0.001 μM or about 0.0001 μM. The administering step can be performed by any acceptable means, including oral, inhalation, topical, intravenous, intraperitoneal, and intramuscular administration.

The following non-limiting examples are illustrative of the present invention:

### Example 1

Inhibition of fibrinolysis by high PAI-1 activity could result in persistence of fibrin matrix and may explain the development of endometriosis in some women, while lower PAI-1 activity may allow rapid fibrin clearance before endometrial fragments can invade and implant. The objective of the study described in this Example was to evaluate the PAI-1 genotype in a group of women with surgically documented endometriosis and a similarly chosen control group.

The following materials and methods were employed in the Study described in this Example.

### Materials and Methods

### Population

Forty-five (45) patients seen from 2004 to 2005 were enrolled in the study. During the study period, 33 women with laparoscopically confirmed endometriosis and 12 controls were included. The study was performed in accordance with the principles of the Declaration of Helsinki, with the protocol approved by the institutional review board and ethical committee of the study site. Informed consents were obtained from all subjects.

### Genotyping

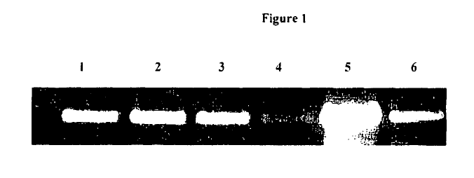

Genomic DNA was prepared from peripheral blood leukocytes of patients using a standard column-extraction technique according to the manufacturer's instruction (Qiagen Inc, Mississauga, On, Canada). The PAI-1 4G/5G-promoter genotype polymorphism was determined by 2 independent polymerase chain reaction (PCR) amplifications. The first PCR was a non-specific reaction using a foreword primer 5′-TAA CCC CTG GTC CCG TTC A-3′ [SEQ ID NO. 3]; and reverse primer 5′-TTT TCC TTT GGC GAA CCA G-3′ [SEQ ID. NO. 4] to amplify the 400 bp segment of the PAI-1 gene promoter containing the area of interest. The 400 bp (FIG. 1) product was purified using the Qiagen DNA purification kit and was sent for automated sequencing in the DNA sequencing facility. The control employed an upstream primer 5′-CCAGACAAGCTTGTTGACACA-3′[SEQ ID NO. 5]; and a downstream primer 5′-ACCTCCATCAAAACGTGGAA-3′[SEQ ID NO. 6] to amplify the 299 bp product.

The second PCR was an allele-specific reaction where the 4G/5G-promoter polymorphism was ascertained by the following primers: (1) insertion 5G allele: 5′-GAC ACG TGG GGG AGT CAG-3′ [SEQ ID. NO. 7]; and (2) deletion 4G allele: 5′-GGA CAC GTG GGG AGT CAG-3′ [SEQ ID. NO. 8], each in combination with (3) a common downstream primer 5′-ACC TCC ATC AAA ACG TGG AA-3′ [SEQ ID. NO. 6] and (4) positive control upstream primer 5′-CCAGACAAGCTTGTTGACACA-3′[SEQ ID NO. 5].

A 25 μL PCR mixture contained, 50 pmol allele-specific primer, 50 pmol common downstream primer, 2.5 pmol control upstream primer, 1×PCR buffer, 2.0 mmol/L magnesium chloride, 0.2 mmol/L dNTPs and 1.25 U Taq polymerase. All of the reagents required for PCR were purchased from Fermentas (Canada). The thermal cycling conditions were 94° C. for 45 seconds, 57° C. for 45 seconds, and 72° C. for 75 seconds for 32 cycles.

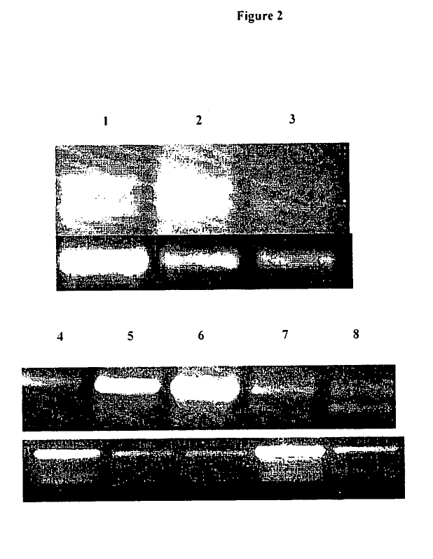

This reaction gave rise to 248 and 299 bp DNA fragments. Electrophoresis of the PCR products was performed on a 1.5 agarose gel and visualized by staining with ethidium bromide, followed by ultraviolet transillumination (FIG. 2). Patients and controls were grouped into 4G/4G, 4G/5G, or 5G/5G genotypes according to the presence of the 248 base pair PCR product generated by the two allele-specific primers. Data including the demographic and clinical variables of patients and controls were collected.

### Results

Demographic variables were comparable between the 2 groups (Table 1). A statistically significant difference in the distribution of PAI-1 genotypes was found between groups. Automated DNA sequencing of the PAI-1 promoter region was used to confirm gel electrophoresis results in every case. Thirty-two of 33 patients with surgically confirmed endometriosis had either the 4G/4G or 4G/5G genotype, a significantly higher percentage than the control group P=0.007, Odds Ratio=0.36 95% Cl (0.16 to 0.79). Twenty-one of 33 women with endometriosis (63.6%) had the 4G/4G genotype, while this genotype was observed in only one out of 12 (8.3%) controls. In contrast, the 5G/5G genotype was found in only one out of 33 (3%) women with endometriosis and in 8 out of 12 (67%) controls. The 4G/5G polymorphism was similarly expressed between the two groups 33.3% (11/33) and 25% (3/12) in the endometriosis patients and controls, respectively (Table 2).

### Discussion

The pathogenesis of endometriosis remains poorly understood. Although retrograde menstruation is observed in more than 90% of women of the reproductive age, only a few of them develop endometriosis [Koninckx, 1980]. An in vitro model for studying endometriosis showed that the presence of fibrin is central to reproduce the initial stages of the disease [Fasciani, 2003]. The presence of the 4G allele in the promoter region of the PAI-1 is associated with hypofibrinolysis and the persistence of fibrin. The frequency of the 4G allele in the PAI-1 promoter region in endometriosis patients was significantly higher than the frequency observed in women without endometriosis. Therefore, the 4G/4G and the 4G/5G genotypes may be involved in susceptibility of patients to endometriosis because of the associated delayed clearance of fibrin from the peritoneal pockets.

Genetic variation in the PAI-1 gene is associated with varying levels of PAI-1 activity in healthy individuals, in patients with coronary artery disease [Ye et al, Thromb Haemost. 1995 September; 74(3):837-4 1], and in patients with diabetes mellitus [Panahloo, Circulation. 1995 Dec. 15; 92(12):3390-3]. In this study, a higher prevalence of PAI-1 4G/4G genotype was also found among patients with endometriosis compared to controls. Dawson et al demonstrated that the 4G allele is associated with an increased transcription rate of PAI-1 in HepG2 cells following interleukin-1 stimulation [Dawson, 1993].

The interaction between the PA/plasmin system and metabolic control is intriguing. Serum PAI-1 activity is found to be positively correlated with metabolic indices, including fasting glucose, cholesterol, triglycerides, and BMI in type 2 diabetic patients [Panahloo, 1995; Mansfield, Thromb Haemost. 1995 September; 74(3):842-7]. This correlation was strongest among the PAI-14G/4G genotype group. Hence, patients with the PAI-14G/4G genotype may be more susceptible to the adverse effect of unsatisfactory metabolic control than others. Consequently, patients with endometriosis should be counseled and offered prophylactic measures to negate their long-term risk of metabolic disorders.

The data described herein show for the first time the link between the fibrinolytic system and endometriosis. This observation could explain why only certain women with retrograde menstruation develop endometriosis while others do not. Furthermore, this novel finding forms the basis of non-invasive diagnosis of women at risk to develop endometriosis.

### Example 2

PAI-1 geneotypes were evaluated in additional women with or without endometriosis. In 81 women (60 with laparoscopically confirmed endometriosis and 21 controls), genomic DNA was extracted from blood and the PAI-1 promoter genotype was determined by PCA amplification of NDA using specific primers for the 4G or 5G allele followed by gel electrophoresis. A portion of the PCR product was purified and sequenced to confirm the gel electrophoresis results. The endometriosis group had significantly higher incidence of 4G/4G and 4G/5G PAI-1 genotypes, associated with increased PA-1 activity, compared to the control group (P=0.007, Odds Ratio+0.36%; 95% CI 0.16 to 0.79). Forty-two of 60 women with endometriosis (70%) had the 4G/4G genotype versus only two out of 21 (9.5%) controls. In contrast, the 5G/5G genotype associated with normal fibrinolysis was found in 1/60 women with endometriosis versus 14/21 (66.6%) controls. A detailed description of the methods and results with additional patients (118 total) is in Example 3. The data for the 81 patients is shown in Table 2.

Therefore, hypofibrinolysis as a result of increased PAI-1 expression, was found significantly more often in women with endometriosis compared to controls. Persistence of fibrin matrix could support the initiation of endometriotic lesions in the peritoneal cavity, explaining why some women with retrograde menstruation develop endometriosis while others do not.

### Example 3

### Summary

The objective of this study was to evaluate PAI-1 genotypes in a group of women with or without endometriosis.

Methods: In 118 women (75 with laparoscopically confirmed endometriosis and 43 controls), genomic DNA was extracted from blood and the PAI-1 promoter genotype was determined by PCR amplification of DNA using specific primers for the 4G or 5G allele followed by gel electrophoresis. A portion of the PCR product was purified and sequenced to confirm the gel electrophoresis results.
Results: Endometriosis was much more likely in patients with 4G/5G (odds ratio 38; 95% confidence interval [CI] 6-229) or 4G/4G (odds ratio 441; 95% CI 53-3,694) compared with 5G/5G PAI-1 genotype. Fifty-two of 75 women with endometriosis (69%, 95% CI 58-79%) had the 4G/4G genotype versus only 5 of 43 (12%; 95% CI 4-25%) controls. In contrast, the 5G/5G genotype associated with normal fibrinolysis was found in 2 of 75 (3%; 95% CI 0-9%) women with endometriosis compared with 24 of 43 (56%; 95% CI 40-71%) controls.
Conclusion: Hypofibrinolysis, associated with the 4G allele of the PAI-1 gene, was found significantly more often in women with endometriosis compared to controls. Persistence of fibrin matrix could support the initiation of endometriotic lesions in the peritoneal cavity, explaining why some women with retrograde menstruation develop endometriosis while others do not.

### Patients

118 Caucasian patients were enrolled in the study. During the study period, 75 women with surgically and histopathologically documented endometriosis and 43 surgically documented normal controls were included. The control group consisted of patients who underwent laparoscopic tubal ligation for sterilization (n=16), patients who underwent tubal reanastomosis to restore fertility (n=5) and patients who underwent diagnostic laparoscopy in the context of infertility evaluation and demonstrated normal pelvis by laparoscopy (n=22). None of the endometriosis group or the control group had or ever had a history of bleeding abnormality. The study was performed in accordance with the principles of the Declaration of Helsinki, with the protocol approved by the institutional review board and ethical committee of Mount Sinai Hospital, Toronto, Canada and the Cleveland Clinic Foundation, Cleveland, Ohio. Informed consents were obtained from all subjects.

### Genotyping

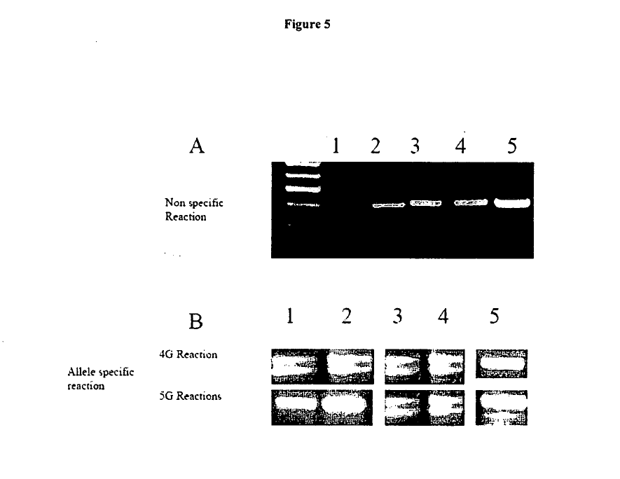

Genomic DNA was prepared from peripheral blood leukocytes of the patients using a standard column-extraction technique according to the manufacturer's instruction (Qiagen Inc, Mississauga, On, Canada). The PAI-1 4G/5G-promoter genotype polymorphism was determined by 2 independent polymerase chain reaction (PCR) amplifications. The first PCR was a non-specific reaction using a foreword primer 5′-TAA CCC CTG GTC CCG TTC A-3′ [SEQ ID NO. 3]; and reverse primer 5′-TTT TCC TTT GGC GAA CCA G-3′ [SEQ ID NO. 4] to amplify the 400 bp segment of the PAI-1 gene promoter containing the area of interest. The 400 bp product (FIG. 5-A) was purified using the Qiagen DNA purification kit and was sent for automated sequencing in the DNA sequencing facility of the Samuel Lunenfeld Research Institute of Mount Sinai Hospital, Toronto, Ontario, Canada.

The second PCR was an allele-specific reaction where the 4G/5G-promoter polymorphism was ascertained by the following primers: (1) insertion 5G allele: 5′-GAC ACG TGG GGG AGT CAG-3′[SEQ ID NO. 7]; and (2) deletion 4G allele: 5′-GGA CAC GTG GGG AGT CAG-3′ [SEQ ID NO. 8], each in combination with (3) a common downstream primer 5′-ACC TCC ATC AAA ACG TGG AA-3′ [SEQ ID NO. 6] and (4) positive control upstream primer 5′-CCAGACAAGGTTGTTGACACA-3′[SEQ ID NO. 5].

The 25 μL PCR mixture contained 50 pmol allele-specific primer, 50 pmol common downstream primer, 2.5 pmol control upstream primer, 1×PCR buffer, 2.0 mmol/L magnesium chloride, 0.2 mmol/L dNTPs and 1.25 U Taq polymerase. All of the reagents required for PCR were purchased from Fermentas (Burlington, On, Canada). The thermal cycling conditions were 94° C. for 45 seconds, 62° C. for 45 seconds, and 72° C. for 75 seconds for 35 cycles.

This PCR reaction gave rise to 248 and 299 bp DNA fragments. Electrophoresis of the PCR products was performed on a 1.5% agarose gel and visualized by staining with ethidium bromide, followed by ultraviolet transillumination (FIG. 5-B). Patients and controls were grouped into 4G/4G, 4G/5G, or 5G/5G genotypes according to the presence of the 248 base pair PCR product generated by the two allele-specific primers. Automated DNA sequencing of the PAI-1 promoter region was used to confirm gel electrophoresis results in every case. Data including the demographic and clinical variables of patients and controls were collected.

### Statistical Analysis

The association between endometriosis and PAI-1 genotype (allele frequencies), clinical center, demographic variables (age, parity, gravidity, height, weight and body mass index [BMI]), infertility and pain was assessed using univariable and multivariable logistic regression. Results are reported as odds ratios and 95% confidence interval [CI]. A multivariable model was formed using backwards variable selection with a significance criterion of P<0.35 in order to adjust the relationship of interest (endometriosis and PAI-1 genotype) for variables with even mild relationships to endometriosis. A Chi-square (χ²) analysis was used in subgroup analyses to assess the association between gene frequency and selected factors. The Mantel-Haenszel test for trend was used to assess an increase or decrease in the proportion with a given condition (such as pain or infertility) for increasing levels of the genotype considered as an ordinal variable (based on the level of 4G presence—none (5G/5G), half (4G/5G) or both (4G/4G).

With the given sample size of 118 patients (75 endometriosis and 43 controls) there was 80% power to detect an odds ratio of about 6 or more in either the 4G/4G or 4G/5G genotype compared with the 5G/5G genotype in the odds of having endometriosis at the 0.05 significance level. The sample size proved sufficient because the observed odds ratios are much larger. SAS statistical software version 9.1 (SAS Institute, Cary, N.C.) was used for all analyses. A significance level of 0.05 was used.

### Results

Demographic variables were comparable between the two groups (Table 3). The severity of endometriosis was graded according to the revised 4-stage American Fertility Society (AFS) scoring system. Of the 75 patients with endometriosis, 38 (51%; 95% CI 39-62%) had early disease (stage I and II) and 37 (49%; 95% CI 38-61%) had advanced disease (stages III and IV). The control group consisted of patients who underwent laparoscopic tubal ligation for sterilization (n=16), patients who underwent tubal reanastomosis to restore fertility (n=5) and patients who underwent diagnostic laparoscopy in the context of infertility evaluation and demonstrated normal pelvis by laparoscopy and random biopsy (n=22). None of the control group showed any laparoscopic evidence of endometriosis confirming the homogeneity of the control group. Mean± standard deviation (SD) age of the study cohort was 37.9±7.5 years, body mass index (BMI) was 24.8±3.6, and parity was 0.5±0.9. No significant associations were observed between endometriosis and age, parity, gravidity or BMI (Table 3).

A statistically significant difference was found in the distribution of PAI-1 genotypes between the two groups on both univariable and multivariable analysis (Table 3). Patients with the 4G/5G genotype were an estimated 18 times more likely (odds ratio 18; 95% CI 4-88) to have endometriosis than those with the 5G/5G genotype, and those with the 4G/4G genotype were an estimated 125 times more likely (95% CI 23-690) than the 5G/5G genotype (P<0.001). On multivariable analysis adjusting for clinical center, gravidity and infertility, the association was even stronger (Table 3). No statistical interactions among factors in the model were found. Also, 73 of 75 patients (97%; 95% CI 91-100%) with surgically confirmed endometriosis had the 4G/4G or the 4G/5G genotype, a significantly higher percentage than the control group [19/43=44%; 95% CI 29-60%), P<0.001; Odds ratio=46; 95% CI 10-213)].

Further analyses were conducted within the endometriosis and control groups to investigate the association between the PAI-1 genotype and the stage of endometriosis and the leading presenting complaint; pelvic pain or infertility (Table 4). When analyzing the genotype as a nominal variable (without ordering among the categories), no significant associations were found. However, pain increased with increasing 4G expression for endometriosis patients (P=0.045) when considering genotype as an ordinal variable (increasing amount of 4G, from 5G/5G (0%) to 4G/5G (19%) to 4G/4G (40%), as did the likelihood of having stage III/IV endometriosis (P=0.06, borderline significant). For control patients, the likelihood of infertility decreased with increased 4G expression (P=0.02).

### Discussion

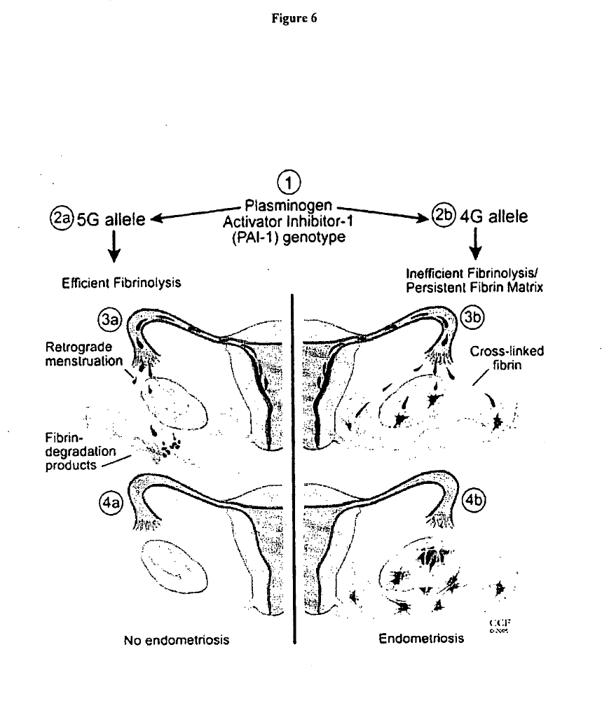

The presence of the 4G allele in the promoter region of the PAI-1 gene is associated with hypofibrinolysis and the persistence of fibrin in vivo. This study found that the frequency of the 4G allele in the PAI-1 promoter region in endometriosis patients was significantly higher than the frequency observed in women without endometriosis. This finding suggests that the 4G/4G and the 4G/5G PAI-1 genotypes could be involved in susceptibility of patients to endometriosis because of the associated delayed clearance of fibrin from peritoneal pockets following retrograde menstruation (FIG. 6).

The association between the PAI-1 genotype and the stage of endometriosis and the leading presenting complaints; pelvic pain or infertility were further evaluated. On analyzing the genotype as a nominal variable, no significant associations were found with either the stage of the disease or the leading presenting symptom. However, when considering genotype as an ordinal variable, pain increased significantly with increasing 4G expression for endometriosis patients. Similarly, there was a trend towards an increased likelihood of having stage III or IV endometriosis (P=0.06, borderline significant). This trend is interesting, as the more fibrinolysis was decreased; the more advanced stage of endometriosis was typically seen. Stage III or IV endometriosis is also associated with more adhesive disease.

It is known that retrograde menstruation is associated with coexistence of fibrin mesh and endometrial fragments. Fibrin formation is ultimately regulated by the proteolytic cleavage of fibrinogen to fibrin, by the activation of factors V, VIII, and XIII and by enhancement of platelet activation [Dennington, Clin Exp Pharmacol Physiol, 1994; 21:349-58]. Immunohistochemical staining has demonstrated that tissue factor is increased in sections of decidualized stromal cells from luteal phase and gestational endometrium [Lockwood, J Clin Endocrinol Metab. 1993; 76:231-6]. Progestin-induced endometrial decidualization has also been associated with stimulation of PAI-1 and suppression of u-PA and t-PA, respectively, both in vivo and in vitro [Lockwood, J Clin Endocrinol Metab. 1995; 80:1100-5]. Finally, thrombin is able to exert mitogenic and angiogenic effects [Dennington, Clin Exp Pharmacol Physiol, 1994; 21:349-58; Naldini, A., et al, Gen Pharmacol 2000; 35:255-9] and, thereby, promote regeneration of the endometrium during the late menstrual and early proliferative phases. Consequently, carriers of the 4G allele, with higher baseline levels of PAI-1, may also produce increased amounts of PAI-1 in response to retrograde menstruation, thereby leading to further inhibition of fibrinolysis. Persistence of fibrin meshes could trap endometrial fragments in peritoneal pockets for a prolonged time. Thus, the results of this study reveal a potential causal mechanism that is likely to contribute to the initiation of endometriotic lesions.

Although endometriosis is frequently associated with extensive peritoneal adhesions [Jirasek, J E. et al., J Reprod Med 1998; 43 suppl: 276-80], the relationship of these adhesions to the initiation or activity of the disease is poorly defined. Peritoneal wounds heal without adhesions in some patients, and others develop severe adhesions from similar surgical procedures. Moreover, adhesions can develop at one surgical site and not in another in the same patient [Chegini N., Front Biosci 2002; 7e91-115]. An experimental study has demonstrated that mice deficient in t-PA were more susceptible to adhesion formation after a surgical insult than wild-type mice [Sulaiman H., et al., Biochem Soc Trans 2002; 30:126-31]. In addition, a recent clinical study demonstrated increased peritoneal adhesions on second-look laparoscopy in endometriosis patients with increased peritoneal fluid PAI-1 activity [Hellebrekers Bw et al., Fertil Steril 2005; 83:122-9]. The findings herein of increased 4G allele of the PAI-1 gene in endometriosis patients may also clarify the potential link between adhesion formation and the pathogenesis of endometriosis.

The data herein show the link between the fibrinolytic system and endometriosis. This may explain why only certain women with retrograde menstruation develop endometriosis while others do not. This novel finding forms the basis of a non-invasive diagnostic test of women at risk to develop endometriosis.

### Example 4

As illustrated in Examples 1, 2, and 3 the presence of a polymorphism in the plasmiogen activator inhibitor-1 (PAI-1) gene, resulting in delayed clearance of fibrin, is significantly increased in women with endometriosis compared to controls. In the present study, another fibrin clearance gene, thrombin-activatable fibrinolysis inhibitor (TAM, was examined to determine if 2 genetic polymorphisms could combine to promote initiation of endometriosis after retrograde menstruation. TAFI attenuates fibrinolysis by cleaving the C-terminal lysine residues from fibrin, thereby inhibiting tissue plasminogen activator (tPA) mediated plasminogen activation. The Thr325Ile mutation in the TAFI gene promoter region is associated with increased antifibrinolytic activity. The objective of this study was to evaluate the TAFI gene polymorphism, Thr325Ile, in endometriosis patients and to match it with the PAI-1 genotype in each patient.

Materials and Methods: Sixty-two women (45 with laparoscopically confirmed endometriosis and 17 control women without endometriosis) were enrolled. Genomic DNA was extracted from peripheral blood leukocytes using a Qiagen kit. Thr325Ile genotypes were determined using PCR and subsequent restriction analysis. The restriction enzyme used was SpeI. The TAFI-325 PCR product was 363 bp, and the Thr allele was digested by SpeI into 118+245 bp whereas the Ile allele was not digested at all by SpeI. The PAI-1 promoter genotype was also determined by PCR amplification of genomic DNA using the allele-specific primers and gel electrophoresis. Each subject was classified as 4G/4G, 4G/5G, or 5G/5G for the PAI-1 genotype, and Thr/Thr, Thr/Ile, or Ile/Ile for the TAFI promoter gene polymorphism.

The following is a detailed PCR procedure that can be employed to identify the Thr325Ile polymorphism in samples. Genomic DNA will be extracted from a buccal smear obtained from each subject. TAFI gene mutations will be detected by polymerase chain reaction-restriction fragment length polymorphism (PCR-RFLP) analysis using the restriction enzyme BbvI for SpeI for TAFI-325. Sequences of the TAFI-325 regions have been retrieved from GenBank. PCR will be carried out in a 25 μl reaction volume containing a standard reaction buffer (1.5 mmol/l MgCl₂, 50 mmol/l KCl, 10 mmol/l Tris-HCl (pH8.3), 200 μmol/l of each dNTP, 10 μmol/l of each primer, 0.5 U Tag DNA polymerase) and 50 ng genomic DNA as a template. The primers will be TAFI325-F (5′-CACAAAGAAAAACAGATCACACAG-3′) [SEQ ID NO. 10], TAFI325-R (5′-AAAGCCACCCAATTGTGATT-3′) [SEQ ID NO. 11]. The protocol will consist of 30 cycles of 94° C. for 30 s, 60° C. for 30 s and 72° C. for 1 min. The TAFI-325 PCR product is 363 bp, and the C (Thr) allele will be digested by SpeI into 118+245 bp fragments whereas the T (Ile) allele will not be digested at all by SpeI. PCR products will be resolved on 2% agarose gels and visualized by ethidium bromide staining. A non-specific PCR will be performed and purified DNA will be sequenced on the first 50 samples to ensure that the restriction enzyme analysis is accurate.

Results: Demographic variables were comparable between the 2 groups. A statistically significant difference in the distribution of PAI-1 genotypes was found between the 2 groups. The distribution of the PAI-1 and TAFI genotype in the endometriosis and the control group are shown in Table 5. Forty-four out of 45 (98%) women with endometriosis had at least one PAI-1 hypofibrinolytic 4G allele. The other hypofibrinoltic TAFI Thr325Ile polymorphism was observed in 44.4% (20/45) of women with endometriosis, and only in 29.4% (5/17) control subjects. On further analysis of the 14 endometriosis patients with the 4G/5G PAI-1 genotype, 71.4% of them (10/14) had the Thr325Ile hypofibrinolytic genotype compared to only 2 PAI-1 heterozgous subjects in the control group.

### Conclusion:

Table 5 shows the distribution of the PAI-1 and TAFI genotypes in the endometriosis and the control group. Of the women with endometriosis, 89% had either a homozygous 4G/4G PAI-1 genotype or combined polymorphism (4G/5G and Thr325Ile TAFI) associated with hypofibrinolysis. In contrast, only 18% of the control women had either type of hypofibrinolytic genotype.

Fibrinolytic system abnormalities may be responsible for persistence of fibrin clots in the peritoneal cavity at the time of retrograde menstruation, and this may be the etiology of the initiation of endometriosis. Of these hypofibrinolytic abnormalties the PAI-14G/4G and 4G/5G genotypes as well as the TAFI Thr325Ile polymorphism have been characterized in women with documented endometriosis.

### Example 5

The prevelance of t-PA-7351C/T polymorphism in endometriosis patients and normal controls will be tested. Tissue plasminogen activator is considered to be the primary mediator of local intravascular fibrinolysis. A low capacity for rapid t-PA release is likely to predispose to hypofibrinolysis. Recently, a single nucleotide polymorphism located at position −7351 within the enhancer region of the t-PA gene was identified and shown to be strongly correlated with endothelial t-PA release rates [Ladenvall P., Thromb Haemost 2000; 84:150-155]. The polymorphism was found to be functional since it occurred within a Sp1 binding site, a factor promoting DNA transcription. The presence of a thymidine (T) base was shown to inhibit Sp1 binding and was associated with less than half the t-PA release observed in those homozygous for the presence of a cytosine (C) base [Ladenvall P., Thromb Haemost 2000; 84:150-155]. The t-PA −7351C/T polymorphism has subsequently been shown to be clinically relevant, having a strong association with first myocardial infarction [Ladenvall P., Thromb Haemost 2002; 87:105-109]. However, its possible relationship to in vivo extravascular fibrinolysis has not been substantiated. A low capacity for rapid t-PA release due to the −7351C/T polymorphism in the tPA gene enhancer may predispose to hypofibrinolysis leading to persistence of fibrin and intiation of endometriosis.

Methodology: Genomic DNA will be isolated from a buccal smear. A 405-bp DNA fragment involving the −7351C/T polymorphism of the tPA gene will be amplified using a sequence-specific PCR method The sequence of the 2 reverse allele-specific primers is 5′-ATGGCTGTGTCTGGGGCG-3′ [SEQ ID NO. 12], 5′-ATGGCTGTGTCTGG-GGCA-3′ [SEQ ID NO. 13], and that of the forward consensusprimer is 5′-ATTGGCGCAAACTCCTCA-3′ [SEQ ID NO. 14). The PCR products will be separated on a 2% agarose gel and the allelic frequency determined. The first 50 sample results will be confirmed by sequencing.
Expected Results The tPA polymorphism is expected to be important in the group of women who have the heterozygous 4G/5G PAI-1 genotype. In this group, the addition of a second or third gene polymorphism causing hypofibrinolysis may be necessary to promote fibrin persistance in the pelvis long enough to allow endometriosis to occur.

### Example 6

The frequency of the C→T and T→C polymorphisms in the nucleotide sequence of exon 6 encoding the kringle domain, and in intron 7, of the uPA gene will be evaluated in subjects with endometriosis and normal controls. Elevated levels of uPA can play a pivotal role in tumour cell spread and metastasis and can be associated with a poorer prognosis. Additionally, the uPA system can regulate other physiological processes important for tumour progression including cell proliferation, migration, adhesion, and angiogenesis. A similar process could happen in patients with endometriosis. Two polymorphism of the uPA gene have been described: a substitution of C for T in the nucleotide sequence of exon 6 encoding the kringle domain (the C→T polymorphism), and a T to C change in intron 7 (the T→C polymorphism). Both result in increased uPA activity.

Methodology: DNA will be extracted from a buccal smear. Genotypes of the C→T polymorphism in the exon 6 encoding the kringle domain of uPA will be determined using the allele specific primers: 5′-GCA GGT GGG CCT AAA GCC-3′ [SEQ ID NO. 15], for the C allele and 5′-GCA GGT GGG CCT AAA GCT-3′ [SEQ ID NO. 16., for the T allele, each in a separate reaction together with the common downstream primer 5′-GGC TCA GAG TCT TTT TGG C-3′ [SEQ ID NO. 17]. A fourth primer: 5′-CTT CAG CAAACG TAC CAT GC-3′ [SEQ ID NO. 18], located upstream of the polymorphic region, will be used as a positive control in the PCR reaction to verify the occurrence of DNA amplification.

Genotypes of the T→C polymorphism in the intron 7 of uPA will be determined using the allele specific primers: 5′-CTA AGC TGT TTG ATG GGT ATC TTC TC-3′ [SEQ ID NO. 19], for the C allele and T 5′-CTA AGC TGT TTG ATG GGT ATC TTC TT-3′ [SEQ ID NO. 20], for the T allele, each in separate reaction together with the common downstream primer 5′-GGC AGC TTC AAC TCA GCT CCA GT-3′ [SEQ ID NO. 21] and a control upstream primer 5″-CGC CTC ATT TCT CCC TCA TCT GC-3′ [SEQ ID NO. 22] to verify the occurrence of DNA amplification in the absence of the allele in the genomic DNA.

The PCR will be carried out in a total volume of 25 μl, containing 50 ng genomic DNA, 10 pmol of each appropriate primer, 200 μM dNTP (Boehringer, Mannheim, Germany), 20 mM Tris-HCl (pH 8.4), 50 mM KCI, 2 mM MgCl₂, and 1 unit of Taq polymerase. The thermal cycling conditions are: 4 min at 95° C., followed by 35 cycles of 30 s at 95° C., 30 s at 60° C. and 1 min at 72° C. The amplified DNA fragments will be separated by a 7% polyacrylamide gel electrophoresis and visualized by ethidium bromide staining. DNA sequencing of the regions containing the polymorphisms will be performed on the first 50 samples to validate both assays.

Expected results: The uPA polymorphism may be important for enhancing invasion and spread of endometriosis, and may be a genetic marker for the severity or aggressiveness of endometriosis.

### Example 7

Following confirmation of the fibrinolytic system gene polymorphisms associated with endometriosis in patients, a diagnostic model will be developed based on the data using ROC curves, predictive values and likelihood ratios. A prospective clinical study will be carried out using the diagnostic model to distinguish which women with pelvic pain have endometriosis or other suspected causes of pelvic pain that require laparoscopy, such as pelvic inflammatory disease, irritable bowel syndrome or ovarian cysts.

Methodology: The prospective study will include patients between 18 and 43 years of age complaining of chronic pelvic pain (>6 months) with/without dysmenorrhea and with/without infertility. All such women referred to the outpatient clinic for diagnostic laparoscopy will be included in the study. All subjects should have regular menstrual cycles (25-35 days), and be in good general health. Cases with suspected endometriomas or other ovarian cysts visible by transvaginal ultrasound examination will be included in the study. Other suspected causes of pelvic pain, such as pelvic infections, and gastrointestinal and genitourinary diseases or the use of any medical therapy for suspected endometriosis, including birth-control pills during the 3 months preceding enrollment, will not be considered exclusion criteria, since the objective is to determine the diagnostic accuracy of the test. None of the patients should have had laparoscopic investigation previously. Buccal smears will be obtained from the patients, DNA will be extracted and fibrinolytic system polymorphisms will be detected using the methodology mentioned herein.

All laparoscopies will be performed by experienced operators, and will be videotaped. Before excision, all areas of typical and atypical endometriosis visualized at laparoscopy will be classified for location and type of lesion. Samples will be taken for histopathological assessment. The samples will be immediately fixed in formalin, and thereafter embedded in paraffin. Afterward, all samples will be cut with a microtome in slices 3- to 4-μm thick, and stained in a standard fashion with hematoxylin-eosin. The diagnosis of endometriosis will be confirmed histopathologically by the presence of glands and stroma. Rare cases where the laparoscopist makes the diagnosis of endometriosis but the pathologist cannot confirm it histologically will be excluded from the study. The same pathologist, who was blind to the laparoscopic classification, will perform all histologic examinations. Staging of the disease will be performed according to the revised American Fertility Association (AFS) classification of endometriosis. After surgery, the revised AFS scores will be recalculated according to the results of histologic examination. The surgical diagnosis will be matched with the diagnosis made based on the detection of the fibrinolytic system gene polymorphisms.

Expected Results: Patients with endometriosis may be predicted from the genotypic analysis of PAI-1, TAFI and tPA. In addition, the severity score on the revised AFS classification may be predicted by the uPA genotype.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the methods etc. which are reported therein which might be used in connection with the invention. Nothing herein is to be construed as an admission that the invention is not entitled to antedate such disclosure by virtue of prior invention.

TABLE 1
Frequency of PAI-1 inhibitor-1 genotype in endometriosis
patients and control

| Genotype | Endometriosis | Control |
|---|---|---|
| 4G/4G | 21/33 (63.3%) | 1/12 (8.3%) |
| 4G/5G | 11/33 (33.3%) | 3/12 (25%) |
| 5G/5G | 1/33 (3%) | 8/12 (67%) |

TABLE 2
Frequency of PAI-1 Genotype in Endometriosis Patients and Controls

| | Endometriosis | Control | |
|---|---|---|---|
| Variable | (n = 60) | (n = 21) | P value |
| | Mean ± SD or % | Mean ± SD or % | |
| Age (years) | 34.0 ± 5.3 | 35.6 ± 5.9 | 0.58 |
| Gravidity | 1.0 ± 1.4 | 2.5 ± 2.7 | 0.15 |
| Parity | 0.7 ± 0.9 | 1.65 ± 1.2 | 0.09 |
| Weight (kg) | 67.2 ± 12.9 | 73.1 ± 14.6 | 0.48 |
| Height (m) | 1.63 ± 0.2 | 1.67 ± 0.09 | 0.17 |
| BMI (kg/m²) | 25.3 ± 4.5 | 26.1 ± 4.05 | 0.16 |
| Infertility | 64% | 28% | <.0001 |
| Pelvic pain | 56% | 0% | <.0001 |
| PAI-1 Genotype * | | | |
| 4G/4G | 42/60 (70.0%) | 2/21 (9.5%) | |
| 4G/5G | 17/60 (28.3%) | 5/21 (23.8%) | |
| 5G/5G | 1/60 (1.6%) | 14/21 (66.6%) | |

* P = 0.007, Odds Ratio = 0.36; 95% CI (0.16 to 0.79).

TABLE 3
Association between PAI-1 genotype and endometriosis and
control groups.

| | | | Odds ratio | |
|---|---|---|---|---|
| | | | (95% | |
| | Endometriosis | Control | Confidence | |
| Variable | (n = 75) | (n = 43) | Interval) | P value |
| Age (yrs) | 37.5 ± 7.4 | 38.5 ± 7.8 | 0.84 (0.51-1.4)^{a} | 0.49 |
| Gravidity | 0.7 ± 1.3 | 1.0 ± 1.2 | 0.86 (0.64-1.1) | 0.30 |
| Parity | 0.4 ± 0.8 | 0.6 ± 1.0 | 0.72 (0.47-1.1) | 0.12 |
| Weight (kg) | 68.2 ± 11.5 | 65.7 ± 7.6 | 1.1 (0.93-1.4)^{b} | 0.20 |
| Height (m) | 1.7 ± 0.1 | 1.6 ± 0.1 | 1.2 (0.73-2.0)^{c} | 0.45 |
| BMI (kg/m²) | 25.0 ± 3.7 | 24.5 ± 3.5 | 1.2 (0.71-2.1)^{d} | 0.48 |
| Infertility | 24 (32) | 21 (49) | 1.4 (0.97-2.1) | 0.07 |
| Pelvic pain | 25 (75) | 0 (0) | N/A | <.001 |
| PAI-1 Genotype univariable analysis | | | | <.001 |
| 5G/5G | 2 (3) | 24 (56) | 1.0 (reference) | — |
| 4G/5G | 21 (28) | 14 (33) | 18 (4-88) | <.001 vs ref |
| 4G/4G | 52 (69) | 5 (12) | 125 (23-690) | <.001 vs ref |
| PAI- 1 Genotype multivariable model | | | | <.001^{e} |
| 4G/5G | 21 (28) | 14 (33) | 38 (6-229) | <.001 vs ref |
| 4G/4G | 52 (69) | 5 (12) | 441 (53-3694) | <.001 vs ref |

BMI, body mass index;
PAI-1, plasminogen activator inhibitor-1,
Data are mean ± standard deviation or n(%) unless otherwise specified.
^{a}per 10 yrs;
^{b}per 10 kg;
^{c}per 10 cm;
^{d}per 5 kg
^{e}Model adjusting for center (P = 0.08), infertility (P = .003 ) and gravidity (P = 0.34)

TABLE 4
Association between PAI-1 genotype and selected factors in
both groups.

| Variable | 4G/4G | 4G/5G | 5G/5G | P ^{a} |
|---|---|---|---|---|
| Endometriosis | | | | |
| Patients (N = 75) | | | | |
| n | 52 | 21 | 2 | |
| Infertility/Pain | | | | 0.28 ^{b} |
| Neither | 9 (17.3) | 3(14.3) | 0 (0) | |
| Pain only | 10 (19.2) | 2 (9.5) | 0 (0) | |
| Infert. only | 22 (42.3) | 14 (66.7) | 2 (10) | |
| Both | 11 (21.2) | 2 (9.5) | 0 (0) | |
| Endom. Pain | 21 (40.0) | 4 (19.0) | 0 (0) | 0.08 ^{b} |
| | | | | 0.045 ^{c} |
| Infertility | 33 (63.5) | 16 (76.2) | 2 (100) | 0.30 ^{b} |
| | | | | 0.16 ^{c} |
| Endom. Stage | | | | |
| Stage I, II | 23 (44.2) | 13 (61.9) | 2 (10) | 0.17 ^{b} |
| Stage III, IV | 29 (55.8) | 8 (38.1) | 0 (0) | 0.06 ^{c} |
| Controls (N = 43) | N = 5 | N = 14 | N = 24 | |
| Infertility | 1 (20.0) | 5 (35.7) | 16 (66.6) | 0.06 |
| | | | | 0.02 ^{c} |
| All (N = 118) | | | | |
| n | 57 | 35 | 26 | |
| Infertility | 34 (60.0) | 21 (60.0) | 18 (69.2) | 0.68 |
| | | | | 0.45 ^{c} |

Data are n (%)
^{a} Chi-square test unless noted;
^{b} not including 5G/5G group since n = 2
^{c} Mantel-Haenszel test for trend

TABLE 5
Distribution of the PAI-1 and TAFI genotypes
in the endometriosis and the control groups.

| | | 4G/4G | 4G/5G | 5G/5G | Total |
|---|---|---|---|---|---|
| Endometriosis | PAI-1 gene | 30/45 | 14/45 | 1/45 | |
| group | | (66.7%) | (31%) | (2.2%) | |
| | TAFI gene | | | | |
| | Thr/Thr | 18/30 | 3/14 | 1/1 | 22/45 |
| | | (60%) | (21.4%) | (100%) | (48.8%) |
| | Thr/Ile | 10/30 | 10/14 | | 20/45 |
| | | (33.3%) | (71.4%) | | (44.4%) |
| | Ile/Ile | 2/30 | 1/14 | | 3/45 |
| | | (6.6%) | (7.2%) | | (6.66%) |
| Control group | PAI-1 gene | 1/17 | 5/17 | 11/17 | |
| | | (5.8%) | (29%) | (64.8%) | |
| | TAFI gene | | | | |
| | Thr/Thr | 1/1 | 2/5 | 6/11 | 9/17 |
| | | (100%) | (40%) | (54.5%) | (52.9%) |
| | Thr/Ile | | 2/5 | 3/11 | 5/17 |
| | | | (40%) | (27.3%) | (29.4%) |
| | Ile/Ile | | 1/5 | 2/11 | 3/17 |
| | | | (20%) | (18.2%) | (17.6%) |

### FULL CITATIONS FOR REFERENCES

1. Sampson J A. Peritoneal endometriosis due to the menstrual dissemination of endometrial tissue into the peritoneal cavity. Am J Obstet Gynecol 1927; 14:422.
2. Sanfilippo J S, Wakim N G, Schikler K N, Yussman M A. Endometriosis in association with uterine anomaly. Am J Obstet Gynecol 1986; 154:39-43.
3. Olive D L, Henderson D Y. Endometriosis and mullerian anomalies. Obstet Gynecol 1987; 69:412-415.
4. Koninckx P R, Heyns W, Verhoeven G, Van Baelen H, Lissens W D, De Moor P, Brosens I A. Biochemical characterization of peritoneal fluid in women during the menstrual cycle. J Clin Endocrinol Metab 1980; 51:1239-1244.
5. Halme J, Hammond M G, Hulka J F, Raj S G, Talbert L M. Retrograde menstruation in healthy women and in patients with endometriosis. Obstet Gynecol 1984; 64:151-154.
6. Liu D T, Hitchcock A. Endometriosis: its association with retrograde menstruation, dysmenorrhoea and tubal pathology. Br J Obstet Gynaecol 1986; 93:859-862.
7. Kruitwagen R F, Poels L G, Willemsen W N, Jap P H, Thomas C M, Rolland R. Retrograde seeding of endometrial epithelial cells by uterine-tubal flushing. Fertil Steril 1991; 56:414-420.
8. Blumenkrantz M J, Gallagher N, Bashore R A, Tenckhoff H. Retrograde menstruation in women undergoing chronic peritoneal dialysis. Obstet Gynecol 1981; 57:667-670.
9. Ridley J. The histogenesis of endometriosis: A review of facts and fancies. Obstet Gynecol Surv 1968; 23.
10. Fasciani A, Bocci G, Xu J, Bielecki R, Greenblatt E, Leyland N, Casper R F. Three-dimensional in vitro culture of endometrial explants mimics the early stages of endometriosis. Fertil Steril 2003; 80:1137-1143.
11. Kruithof E K. Plasminogen activator inhibitors—a review. Enzyme 1988; 40:113-121.
12. Lindahl T L, Ohlsson P I, Wiman B. The mechanism of the reaction between human plasminogen-activator inhibitor 1 and tissue plasminogen activator. Biochem J 1990; 265:109-113.
13. Owensby D A, Morton P A, Wun T C, Schwartz A L. Binding of plasminogen activator inhibitor type-1 to extracellular matrix of Hep G2 cells. Evidence that the binding protein is vitronectin. J Biol Chem 1991; 266:4334-4340.
14. Kooistra T, Sprengers E D, van Hinsbergh V W. Rapid inactivation of the plasminogen-activator inhibitor upon secretion from cultured human endothelial cells. Biochem J 1986; 239:497-503.
15. Dawson S, Hamsten A, Wiman B, Henney A, Humphries S. Genetic variation at the plasminogen activator inhibitor-1 locus is associated with altered levels of plasma plasminogen activator inhibitor-1 activity. Arterioscler Thromb 1991; 11:183-190.
16. Dawson S J, Wiman B, Hamsten A, Green F, Humphries S, Henney A M. The two allele sequences of a common polymorphism in the promoter of the plasminogen activator inhibitor-1 (PAI-1) gene respond differently to interleukin-1 in HepG2 cells. J Biol Chem 1993; 268:10739-10745.
17. Eriksson P, Kallin B, van 't Hooft F M, Bavenholm P, Hamsten A. Allele-specific increase in basal transcription of the plasminogen-activator inhibitor 1 gene is associated with myocardial infarction. Proc Natl Acad Sci USA 1995; 92:1851-1855.
18. Henry M, Tregouet D A, Alessi M C, Aillaud M F, Visvikis S, Siest G, Tiret L, et al. Metabolic determinants are much more important than genetic polymorphisms in determining the PAI-1 activity and antigen plasma concentrations: a family study with part of the Stanislas Cohort. Arterioscler Thromb Vasc Biol 1998; 18:84-91.
19. Glueck C J, Phillips H, Cameron D, Wang P, Fontaine R N, Moore S K, Sieve-Smith L, et al. The 4G/4G polymorphism of the hypofibrinolytic plasminogen activator inhibitor type I gene: an independent risk factor for serious pregnancy complications. Metabolism 2000; 49:845-852.
20. Dennington P M, Berndt M C. The thrombin receptor. Clin Exp Pharmacol Physiol 1994; 21:349-358.
21. Lockwood C J, Nemerson Y, Guller S, Krikun O, Alvarez M, Hausknecht V, Gurpide E, et al. Progestational regulation of human endometrial stromal cell tissue factor expression during decidualization. J Clin Endocrinol Metab 1993; 76:231-236.
22. Lockwood C J, Krikun G, Papp C, Aigner S, Schatz F. Biological mechanisms underlying the clinical effects of RU 486: modulation of cultured endometrial stromal cell plasminogen activator and plasminogen activator inhibitor expression. J Clin Endocrinol Metab 1995; 80:1100-1105.
23. Naldini A, Carney D H, Pucci A, Pasquali A, Carraro F. Thrombin regulates the expression of proangiogenic cytokines via proteolytic activation of protease-activated receptor-1. Gen Pharmacol 2000; 35:255-259.
24. Jirasek J E, Henzl M R, Uher J. Periovarian peritoneal adhesions in women with endometriosis. Structural patterns. J Reprod Med 1998; 43:276-280.
25. Chegini N. Peritoneal molecular environment, adhesion formation and clinical implication. Front Biosci 2002; 7:e91-115.
26. Sulaiman H, Dawson L, Laurent G J, Bellingan G J, Herrick S E. Role of plasminogen activators in peritoneal adhesion formation. Biochem Soc Trans 2002; 30:126-131.
27. Hellebrekers B W, Emeis J J, Kooistra T, Trimbos J B, Moore N R, Zwinderman K H, Trimbos-Kemper T C. A role for the fibrinolytic system in postsurgical adhesion formation. Fertil Steril 2005; 83:122-129.

1. A method for treating endometriosis in a subject, said method comprising reducing hypofibrinolysis or reducing fibrin matrix in peritoneal pockets in endometrium in the subject.

2. The method according to claim 1, comprising modulating plasminogen-activator inhibitor 1 (PAI-1), tissue plasminogen (tPA), urokinase plasminogen activator (uPA), thrombin-activatable fibrinolysis inhibitor (TAFI), or combinations thereof.

3. The method according to claim 2, comprising reducing or inhibiting:
(i) PAI-1 bp administering an antagonist of PAI-1,
(ii) uPA by administering an antagonist of uPA,
(iii) TAFI by administering an antagonist of TAFI, or
(iv) combinations of (i)-(iii).

4. The method according to claim 2, comprising enhancing or increasing tPA.

5. A method for treating endometriosis, said method comprising:
analyzing nucleic acids in a sample of biological material from a subject to detect the presence of at least one polymorphism in a gene of the fibrinolytic pathway associated with endometriosis; and
treating the subject to counteract the effect of detected polymorphism.

6. A method for the prophylactic treatment of a subject with a genetic predisposition to endometriosis, said method comprising:
analyzing nucleic acids in a sample of biological material from the subject to detect the presence of at least one polymorphism in a gene of the fibrinolytic pathway associated with endometriosis; and
treating the subject.

7. A method for identifying a polymorphism in at least one a gene of the fibrinolytic pathway that correlates with endometriosis, said method comprising:
obtaining the sequence for the gene from a group of patients with endometriosis,
identifying a site of at least one polymorphism in the gene, and
determining genotypes at the site for individual patients in the group.

8. A method for diagnosing a genetic susceptibility for endometriosis in a subject, said method comprising:
analyzing the nucleic acids in a biological sample from the subject to detect the presence or absence of one or more polymorphisms in one or more genes of a fibrinolytic pathway of the subject, wherein the polymorphisms are associated with a genetic predisposition for endometriosis.

9. The method according to claim 5, wherein the gene is a PAI-1 gene, a tPA gene, a uPA gene, a TAFI gene, or a combination thereof.

10. A method for diagnosing endometriosis in a subject having, at risk of developing, or susceptible to endometriosis, said method comprising:
determining the genotype of one or more polymorphisms in a PAI-1 gene, tPA gene, uPA gene, TAFI gene, or a combination thereof associated with endometriosis, and
optionally comparing the genotype with known genotypes which are indicative of endometriosis.

11. The method according to claim 10, wherein the genotype is determined at a combination of multiple polymorphism sites within the promoter region or outside the promoter region of a PAI-1 gene.

12. The method according to claim 11, wherein a polymorphism in a PAI-1 gene is a 4G polymorphism in the promoter region of the PAI-1 gene.

13. The method according to claim 12, wherein the polymorphism is defined by a mutation at the base occupying position 837 of SEQ ID NO. 1.

14. The method according to claim 13, wherein the mutation at position 837 is the absence of a G.

15. The method according to claim 10, wherein a polymorphism in a TAFI gene is a polymorphism in a sequence encoding an amino acid at position 325 of TAFI.

16. The method according to claim 15, wherein the polymorphism is a C to T substitution at position 1064 of SEQ ID NO. 9.

17. The method according to claim 10, wherein the polymorphism in a tPA gene is a −7351C/T tPA polymorphism.

18. The method according to claim 10, wherein the polymorphism in an uPA gene is a C→T polymorphism in the nucleotide sequence of exon 6 encoding the kringle domain of uPA.

19. A The method according to claim 10, wherein the polymorphism in an uPA gene is a T→C polymorphism in the nucleotide sequence of intron 7 of an uPA gene.

20. A method for diagnosing a single nucleotide polymorphism in a PAI-1 gene in a biological sample from a human having endometriosis, said method comprising:
determining the nucleic acid at the position that correlates with position 837 of SEQ ID NO. 1.

21. A method for determining an individual's risk for endometriosis, said method comprising:
determining an individual's genotype at one or more polymorphic sites in a PAI-1 gene, wherein a first 4G/4G genotype at position 837 of SEQ ID NO.1 is statistically associated with an increased risk for endometriosis as compared to a second 5G/5G genotype at the polymorphic site.

22. The method according to claim 21, further comprising determining a Thr325Ile polymorphism for a TAFI gene, wherein a Thr325Ile polymorphism is statistically associated with an increased risk for endometriosis.

23. A method for determining the efficacy of treating a patient with endometriosis based on genotype, said method comprising:
(a) determining the genotype for one or more polymorphism sites in a gene of the fibrinolytic pathway for a group of patients receiving a treatment;
(b) sorting the group of patients into subgroups based on genotype;
(c) identifying correlations between the subgroups and the efficacy of the treatment in the patient;
(d) determining the genotype for the same polymorphism sites in the genes of the patient and determining the efficacy of the treatment for the patient based on a comparison of the genotype with the correlations identified in (c).

24. The method according to claim 23, wherein the gene is a PAI-1 gene, tPA gene, uPA gene, TAFI gene, or a combination thereof.

25. A kit for carrying out a method of claim 1.
